(19) United States
(12) Patent Application Publication
(10) Pub. No.: US 2023/0357275 A1
(43) Pub. Date: Nov. 9, 2023

(54) SPIRO-SULFONAMIDE DERIVATIVES AS INHIBITORS OF MYELOID CELL LEUKEMIA-1 (MCL-1) PROTEIN

(71) Applicant: Prelude Therapeutics, Incorporated, Wilmington, DE (US)

(72) Inventors: Jincong Zhuo, Garnet Valley, PA (US); Ganfeng Cao, Chadds Ford, PA (US); Andrew Paul Combs, Kennett Square, PA (US); Qun Li, Newark, DE (US); Huaping Zhang, Newark, DE (US)

(21) Appl. No.: 17/998,518

(22) PCT Filed: May 13, 2021
(86) PCT No.: PCT/US2021/032263

§ 371 Date: Nov. 11, 2022

Related U.S. Application Data
(60) Provisional application No. 63/024,110, filed on May 13, 2020.

(51) Int. Cl. C07D 515/10; A61P 35/00

(52) U.S. Cl. CPC C07D 515/10; A61P 35/00; C07B 2200/13

(57) ABSTRACT

The disclosure is directed to crystalline forms of the compound of Formula I: Formula (I), and pharmaceutically acceptable salts thereof. Pharmaceutical compositions comprising compounds of Formula I as well as methods of their use and preparation, are also described.

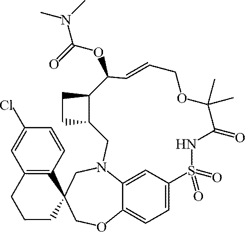 [SMILES: CN(C)C(=O)O[C@H]1/C=C/COC(C)(C)C(=O)NS(=O)(=O)c2ccc3c(c2)N(C[C@@H]2CC[C@H]21)C[C@@]1(CCCc2cc(Cl)ccc21)CO3]

### CROSS-REFERENCE TO RELATED APPLICATION

This application claims the benefit of priority to U.S. Provisional Pat. Application No. 63/024,110, filed on May 13, 2020, the entirety of which is incorporated by reference herein.

### TECHNICAL FIELD

The disclosure is directed to MCL-1 inhibitors and methods of their use.

### BACKGROUND

Apoptosis (programmed cell death) is a highly conserved cellular process that is required for embryonic development and normal tissue homeostasis (Ashkenazi A. et al., Nat. Rev. Drug Discov. 2017, 16, 273-284). Apoptotic-type cell death involves morphological changes such as condensation of the nucleus, DNA fragmentation as well as biochemical phenomena such as the activation of caspases which cause damage to key structural components of the cell, resulting in its disassembly and death. Regulation of the process of apoptosis is complex and involves the activation or repression of several intracellular signaling pathways (Cory S. et al., Nature Review Cancer 2002, 2, 647-656; Thomas L. W. et al., FEBS Lett. 2010, 584, 2981-2989; Adams J. M. et al., Oncogene 2007, 26, 1324-1337)

The Bcl-2 protein family, which includes both pro-apoptotic and anti-apoptotic members, plays a pivotal role in the regulation of the apoptosis process (Youle R. J. et al., Nat. Rev. Mol. Cell Biol. 2008, 9, 47-59; Kelly G. L. et al., Adv. Cancer Res. 2011, 111, 39-96). Bcl-2, Bcl-XL, Bcl-W, Mcl-1 and A1 are anti-apoptotic proteins and they share a common BH regions. In contrast, the pro-apoptotic family members are divided into two groups. The multi-region pro-apoptotic proteins, such as Bax, Bak and Bok, are conventionally thought to have BH1-3 regions, whereas the BH3-only proteins are proposed to share homology in the BH3 region only. Members of BH3-only proteins include Bad, Bim, Bid, Noxa, Puma, Bik/Blk, Bmf, Hrk/DP5, Beclin-1 and Mule (Xu G. et al., Bioorg. Med. Chem. 2017, 25, 5548-5556; Hardwick J. M. et al., Cell. 2009, 138, 404; Reed J. C., Cell Death Differ. 2018, 25, 3-6; Kang M. H. et al., Clin Cancer Res 2009, 15, 1126-1132). The pro-apoptotic members (such as BAX and BAK), upon activation, form a homo-oligomer in the outer mitochondrial membrane that leads to pore formation and the escape of mitochondrial contents, a step into triggering apoptosis. Antiapoptotic members of the Bcl-2 family (such as Bcl-2, Bel -XL, and Mcl-1) block the activity of BAX and BAK. In normal cells, this process is tightly regulated. Abnormal cells can dysregulate this process to avoid cell death. One of the ways that cancer cells can accomplish this is by upregulating the antiapoptotic members of the Bcl-2 family of proteins. Overexpression or up-regulation of the anti-apoptotic Bcl-2 family proteins enhance cancer cell survival and cause resistance to a variety of anticancer therapies.

Aberrant expression or function of the proteins responsible for apoptotic signaling contributes to numerous human pathologies including auto-immune diseases, neurodegeneration (such as Parkinson’s disease, Alzheimer’s disease and ischaemia), inflammatory diseases, viral infections and cancer (such as colon cancer, breast cancer, small-cell lung cancer, non-small-cell lung cancer, bladder cancer, ovarian cancer, prostate cancer, chronic lymphoid leukemia, lymphoma, myeloma, acute myeloid leukemia, pancreatic cancer, etc.) (Hanahan D. et al., Cell 2000, 100. 57-70). Herein, it is prospective to target key apoptosis regulators for cancer treatment (Kale J. et al., Cell Death Differ. 2018, 25, 65-80; Vogler M. et al., Cell Death Differ. 2009, 16, 360-367).

By overexpressing one or more of these pro-survival proteins, cancer cells can evade elimination by normal physiological processes and thus gain a survival advantage. Myeloid Cell Leukemia-1 (Mcl-1) is a member of the pro-survival Bcl-2 family of proteins. Mcl-1 has the distinct trait of being essential for embryonic development as well as the survival of all hematopoietic lineages and progenitor populations. Mcl-1 is one of the most common genetic aberrations in human cancer and is highly expressed in many tumor types. Mcl-1 overexpression in human cancers is associated with high tumor grade and poor survival (Beroukhim R. et al., Nature 2010, 463, 899-905). Mcl-1 overexpression prevents cancer cells from undergoing programmed cell death (apoptosis), allowing the cells to survive despite widespread genetic damage. Further, its amplification is associated with both intrinsic and acquired resistance to a wide variety of antitumorigenic agents including chemotherapeutic agents such as microtubule binding agents, paclitaxel and gemcitabine, as well as apoptosis-inducing agents such as TRAIL, the Bcl-2 inhibitor, venetoclax, and the Bcl-2/Bcl-XL dual inhibitor navitoclax. Not only do gene silencing approaches that specifically target Mcl-1 circumvent this resistance phenotype, but certain cancer cell types frequently undergo cell death in response to Mcl-1 silencing, indicating a dependence on Mcl-1 for survival. Consequently, approaches that inhibit Mcl-1 function are of considerable interest for cancer therapy (Wertz I. E et al., Nature 2011, 471, 110-114; Zhang B. et al., Blood 2002, 99, 1885-1893).

### SUMMARY

The disclosure is also directed to crystalline forms of [(3R,6R,7S,8E,22S)-6′-Chloro-12,12-dimethyl-13,15,15-trioxo-spiro[11,20-dioxa-15-thia-1,14-diazatetracyclo[14.7.2.03,6.019,24]-pentacosa-8,16,18,24-tetraene-22,1′-tetralin]-7-yl] N,N-dimethylcarbamate, i.e., the compound of Formula I,

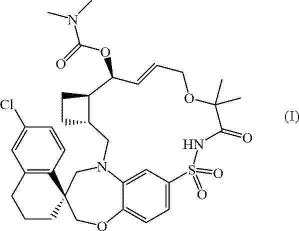 [SMILES: CN(C)C(=O)O[C@H]1/C=C/COC(C)(C)C(=O)NS(=O)(=O)c2ccc3c(c2)N(C[C@@H]2CC[C@H]21)C[C@@]1(CCCc2cc(Cl)ccc21)CO3]

The disclosure is also directed to pharmaceutical compositions containing such forms and methods of use of such forms are also described.

The disclosure is also directed to pharmaceutically acceptable salts of the compound of Formula I.

The disclosure is also directed to choline, benzathine, imidazole, piperazine, piperidine, (S)-(-)-α-methylbenzylamine, ethylenediamine, potassium, and 4-((2-aminoethyl)amino)-4-methylpentan-2-one salts of Formula I.

Crystalline forms of such salts, as well as pharmaceutical compositions containing such salts and methods of use of such salts are also described.

### BRIEF DESCRIPTION OF THE DRAWINGS

FIG. 1 shows an XRPD of Formula I-Form I.

FIG. 2 shows a DSC thermogram of Formula I-Form I.

FIG. 3 shows a TGA profile of Formula I-Form I.

FIGS. 4A and 4B show a DVS profile of Formula I-Form I.

FIG. 5 shows an XRPD of Formula I-Form I before (top) and after (bottom) DVS.

FIG. 6 shows an XRPD of Formula I-Form II.

FIG. 7 shows a DSC thermogram of Formula I-Form II.

FIG. 8 shows an XRPD of a choline salt of Formula I.

FIG. 9 shows a DSC thermogram of a choline salt of Formula I.

FIG. 10 shows a TGA profile of a choline salt of Formula I.

FIG. 11 shows an NMR spectrum (600 MHz in CDCl₃) of a choline salt of Formula I.

FIG. 12 shows an XRPD of a benzathine salt of Formula I.

FIG. 13 shows a DSC thermogram of a benzathine salt of Formula I.

FIG. 14 shows a TGA profile of a benzathine salt of Formula I.

FIG. 15 shows an NMR spectrum (600 MHz in CDCl₃) of a benzathine salt of Formula I.

FIG. 16 shows an XRPD of an imidazole salt of Formula I.

FIG. 17 shows a DSC thermogram of an imidazole salt of Formula I.

FIG. 18 shows a TGA profile of an imidazole salt of Formula I.

FIG. 19 shows an NMR spectrum (600 MHz in CDCl₃) of an imidazole salt of Formula I.

FIG. 20 shows an XRPD of a piperazine salt of Formula I (Form 1).

FIG. 20A shows an XRPD of a piperazine salt of Formula I (Form 2)

FIG. 20B shows an XRPD of a piperazine salt of Formula I (Form 3)

FIG. 21 shows a DSC thermogram of a piperazine salt of Formula I (Form 1).

FIG. 21A shows a DSC thermogram of a piperazine salt of Formula I (Form 2).

FIG. 22 shows a TGA profile of a piperazine salt of Formula I (Form 1).

FIG. 23 shows an NMR spectrum (600 MHz in CDCl₃) of a piperazine salt of Formula I (Form 1).

FIG. 24 shows an XRPD of a piperidine salt of Formula I (Form 1).

FIG. 24A shows an XRPD of a piperidine salt of Formula I (Form 2).

FIG. 25 shows a DSC thermogram of a piperidine salt of Formula I (Form 1).

FIG. 26 shows a TGA profile of a piperidine salt of Formula I (Form 1).

FIG. 27 shows an NMR spectrum (600 MHz in CDCl₃) of a piperidine salt of Formula I (Form 1).

FIG. 28 shows an XRPD of a potassium salt of Formula I.

FIG. 29 shows a DSC thermogram of a potassium salt of Formula I.

FIG. 30 shows an XRPD of a (S)-(-)-α-Methylbenzylamine salt of Formula I.

FIG. 31 shows a DSC thermogram of a (S)-(-)-α-Methylbenzylamine salt of Formula I.

FIG. 32 shows an XRPD of an ethylenediamine salt of Formula I (Form 1).

FIG. 32A shows an XRPD of an ethylenediamine salt of Formula I (Form 2).

FIG. 33 shows NMR Spectrum of an ethylenediamine salt of Formula I (Form 1).

FIG. 34 shows an XRPD of a 4-((2-aminoethyl)amino)-4-methylpentan-2-one salt of Formula I.

FIG. 35 shows a DSC thermogram of a 4-((2-aminoethyl)amino)-4-methylpentan-2-one salt of Formula I.

FIG. 36 shows a TGA profile of a 4-((2-aminoethyl)amino)-4-methylpentan-2-one salt of Formula I.

FIG. 37 shows an NMR spectrum (600 MHz in CDCl₃) of a 4-((2-aminoethyl)amino)-4-methylpentan-2-one salt of Formula I.

### DETAILED DESCRIPTION OF ILLUSTRATIVE EMBODIMENTS

The disclosure may be more fully appreciated by reference to the following description, including the following definitions and examples. Certain features of the disclosed compositions and methods which are described herein in the context of separate aspects, may also be provided in combination in a single aspect. Alternatively, various features of the disclosed compositions and methods that are, for brevity, described in the context of a single aspect, may also be provided separately or in any subcombination.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, e.g., in humans.

“Pharmaceutically acceptable salt” refers to a salt of a compound of the disclosure that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, and polyethylene glycols.

A “solvate” refers to a physical association of a compound of Formula I with one or more solvent molecules.

“Subject” includes humans. The terms “human,” “patient,” and “subject” are used interchangeably herein.

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

“Compounds of the present disclosure,” and equivalent expressions, are meant to embrace the compound of Formula I as well as the pharmaceutically acceptable salts, where the context so permits.

As used herein, the term “isotopic variant” refers to a compound that contains proportions of isotopes at one or more of the atoms that constitute such compound that is greater than natural abundance. For example, an “isotopic variant” of a compound can be radiolabeled, that is, contain one or more radioactive isotopes, or can be labeled with non-radioactive isotopes such as for example, deuterium (²H or D), carbon-13 (¹³C), nitrogen-15 (¹⁵N), or the like. It will be understood that, in a compound where such isotopic substitution is made, the following atoms, where present, may vary, so that for example, any hydrogen may be ²H/D, any carbon may be ¹³C, or any nitrogen may be ¹⁵N, and that the presence and placement of such atoms may be determined within the skill of the art.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers,” for example, diastereomers, enantiomers, and atropisomers. The compounds of this disclosure may possess one or more asymmetric centers; such compounds can therefore be produced as individual (R)-or (S)-stereoisomers at each asymmetric center, or as mixtures thereof. Unless indicated otherwise, the description or naming of a particular compound in the specification and claims is intended to include all stereoisomers and mixtures, racemic or otherwise, thereof. Where one chiral center exists in a structure, but no specific stereochemistry is shown for that center, both enantiomers, individually or as a mixture of enantiomers, are encompassed by that structure. Where more than one chiral center exists in a structure, but no specific stereochemistry is shown for the centers, all enantiomers and diastereomers, individually or as a mixture, are encompassed by that structure. The methods for the determination of stereochemistry and the separation of stereoisomers are well-known in the art.

See, e.g., U.S. Pat. Application No. 16/679,105.

In some aspects, the disclosure is directed to a crystalline form of the compound of Formula I,

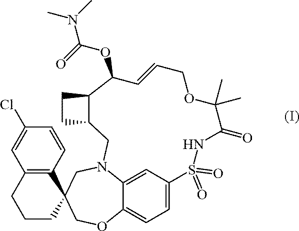 [SMILES: CN(C)C(=O)O[C@H]1/C=C/COC(C)(C)C(=O)NS(=O)(=O)c2ccc3c(c2)N(C[C@@H]2CC[C@H]21)C[C@@]1(CCCc2cc(Cl)ccc21)CO3]

In some embodiments, the disclosure is directed to crystalline form I of the compound of Formula I (Formula I-Form I). In some embodiments, Formula I-Form I is substantially free of any other solid form of Formula I.

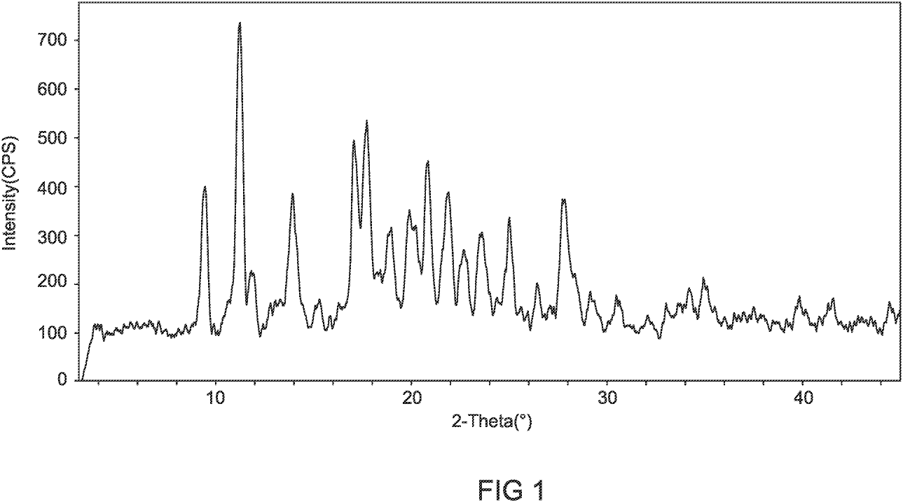

In some embodiments, Formula I-Form I exhibits an XRPD substantially as shown in FIG. 1. The XRPD of Formula I-Form I shown in FIG. 1 comprises reflection angles (degrees 2-theta ± 0.2 degrees 2-theta), line spacings (d values), and relative intensities as shown in Table 1:

TABLE 1

XRPD Data for crystalline form of Formula I-Form I shown in FIG. 1 .

| Angle (degrees 2-theta ± 0.2 degrees 2-theta) | d Value (Å) | Relative Intensity |
|---|---|---|
| 3.98 | 22.1822 | 17.8 |
| 9.44 | 9.3609 | 47.2 |
| 11.22 | 7.8795 | 100 |
| 11.86 | 7.4558 | 18.7 |
| 13.94 | 6.3477 | 42.5 |
| 15.3 | 5.7863 | 9 |
| 17.06 | 5.193 | 52.6 |
| 17.72 | 5.0012 | 57.3 |
| 18.96 | 4.6768 | 20.2 |
| 19.9 | 4.4579 | 29.8 |
| 20.82 | 4.2629 | 45.8 |
| 21.86 | 4.0624 | 36.3 |
| 22.68 | 3.9174 | 17.9 |
| 23.62 | 3.7636 | 25.7 |
| 25 | 3.5588 | 32.6 |
| 26.44 | 3.3682 | 12.8 |
| 27.76 | 3.211 | 38.8 |
| 29.178 | 3.058 | 10.1 |
| 30.538 | 2.9249 | 10 |
| 33.057 | 2.7075 | 6.5 |
| 34.239 | 2.6167 | 12.8 |
| 34.94 | 2.5658 | 12.6 |
| 39.82 | 2.2619 | 10.1 |
| 41.559 | 2.1712 | 9.5 |

In some embodiments of the present disclosure, Formula I-Form I is characterized by an XRPD pattern comprising a peak at one of the angles listed in Table 1. In other aspects, Formula I-Form I is characterized by an XRPD pattern comprising more than one peak at one of the angles listed in Table 1 above. In other aspects, Formula I-Form I is characterized by an XRPD pattern comprising two peaks selected from the angles listed in Table 1 above. In other aspects, Formula I-Form I is characterized by an XRPD pattern comprising three peaks selected from the angles listed in Table 1 above. In other aspects, Formula I-Form I is characterized by an XRPD pattern comprising four peaks selected from the angles listed in Table 1 above. In other aspects, Formula I-Form I is characterized by an XRPD pattern comprising five peaks selected from the angles listed in Table 1 above. In other aspects, Formula I-Form I is characterized by an XRPD pattern comprising six peaks selected from the angles listed in Table 1 above. In other aspects, Formula I-Form I is characterized by an XRPD pattern comprising seven peaks selected from the angles listed in Table 1 above. In other aspects, Formula I-Form I is characterized by an XRPD pattern comprising eight peaks selected from the angles listed in Table 1 above. In other aspects, Formula I-Form I is characterized by an XRPD pattern comprising nine peaks selected from the angles listed in Table 1 above. In other aspects, Formula I-Form I is characterized by an XRPD pattern comprising ten peaks selected from the angles listed in Table 1 above. In other aspects, Formula I-Form I is characterized by an XRPD pattern comprising more than ten peaks selected from the angles listed in Table 1 above.

In some embodiments, Formula I-Form I is characterized by an XRPD pattern comprising a peak 11.2, 13.9, 17.1, 17.7, and 20.8 degrees ± 0.2 degrees 2-theta. In other embodiments, Formula I-Form I is characterized by an XRPD pattern comprising peaks at 9.4, 11.2, 13.9, 17.1, and 17.7 degrees ± 0.2 degrees 2-theta. In other embodiments, Formula I-Form I is characterized by an XRPD pattern comprising peaks at 17.1, 17.7, 20.8, and 21.9 degrees ± 0.2 degree 2-theta. In other embodiments, Formula I-Form I is characterized by an XRPD pattern comprising peaks at 13.9, 17.1, 17.7, 20.8, and 21.9 degrees ± 0.2 degree 2-theta. In other embodiments, Formula I-Form I is characterized by an XRPD pattern comprising peaks at 11.2, 13.9, 17.1, 17.7, 20.8, 21.9, and 25.0 degrees ± 0.2 degree 2-theta.. In other embodiments, Formula I-Form I is characterized by an XRPD pattern comprising peaks at 9.4, 11.2, 13.9, 17.1, 17.7, 20.8, 21.9, 25.0, and 27.8 degrees ± 0.2 degree 2-theta.

In some embodiments of the present disclosure, Formula I-Form I is characterized by an XRPD pattern comprising peaks at two or more of 9.4, 11.2, 13.9, 17.1, 17.7, 20.8, 21.9, 25.0, and 27.8 degrees ± 0.2 degrees 2-theta.

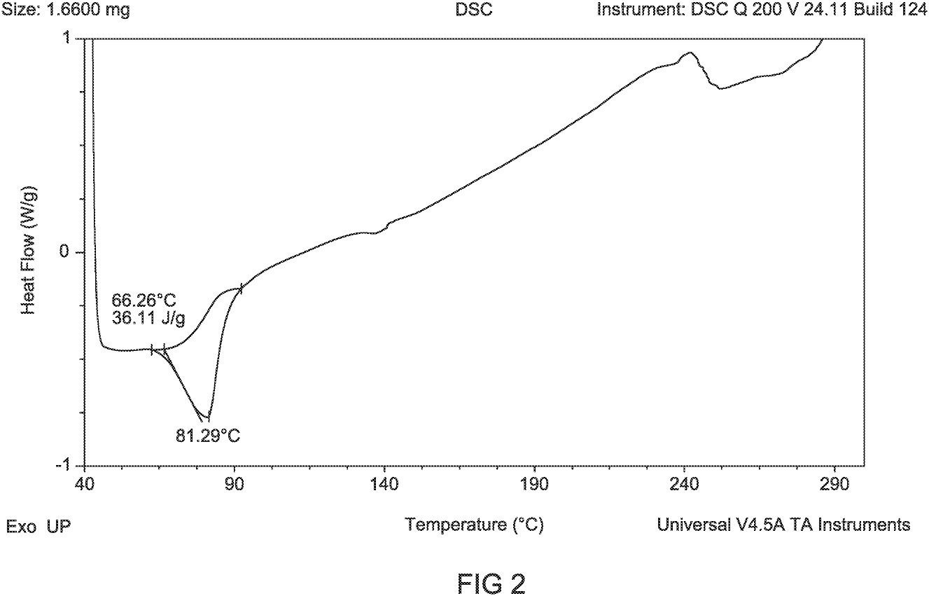

In some embodiments, Formula I-Form I can be characterized by a DSC thermogram substantially as shown in FIG. 2. As FIG. 2 shows, Formula I-Form I produced an endothermic peak at 81.29° C., with a peak onset temperature of 66.26° C., and an enthalpy of melting of 36.11 J/g, when heated at a rate of 10° C./min. In some embodiments of the present disclosure, Formula I-Form I is characterized by a DSC thermogram comprising an endothermic peak at about 81° C. In other embodiments of the present disclosure, Formula I-Form I is characterized by a DSC enthalpy of melting of about 36 J/g.

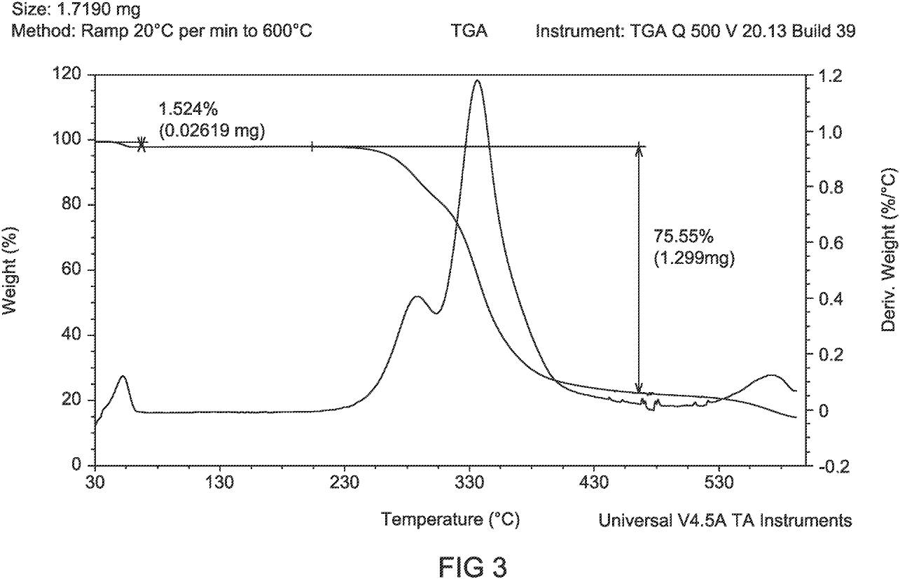

In some embodiments, Formula I-Form I can be characterized by a TGA profile substantially as shown in FIG. 3 when heated at a rate of 20° C./min. As FIG. 3 shows, Formula I-Form I lost about 76% of its weight upon heating to about 430° C.

In some embodiments of the present disclosure, Formula I-Form I is characterized by an XRPD pattern comprising peaks at one or more of 9.4, 11.2, 13.9, 17.1, 17.7, 20.8, 21.9, 25.0, and 27.8 degrees ± 0.2 degrees 2-theta, and a DSC thermogram comprising an endothermic peak at about 81° C. when heated at a rate of 10° C./min.

In some embodiments, the disclosure is directed to crystalline Form II of the compound of Formula I (Formula I-Form II). In some embodiments, Formula I-Form II is substantially free of any other solid form of Formula I.

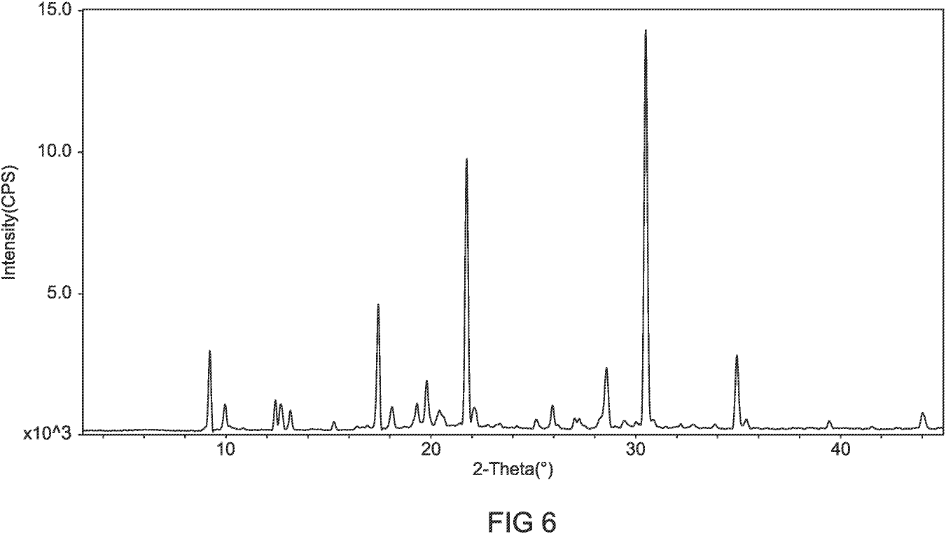

In some embodiments, Formula I-Form II exhibits an XRPD substantially as shown in FIG. 6. The XRPD of Formula I-Form II shown in FIG. 6 comprises reflection angles (degrees 2-theta ± 0.2 degrees 2-theta), line spacings (d values), and relative intensities as shown in Table 2:

TABLE 2

XRPD Data for crystalline form of Formula I-Form II shown in FIG. 6 .

| Angle (degrees 2-theta ± 0.2 degrees 2-theta) | d Value (Å) | Relative Intensity |
|---|---|---|
| 9.2 | 9.6046 | 19.7 |
| 9.941 | 8.8905 | 6.7 |
| 12.644 | 6.9953 | 6.3 |
| 13.139 | 6.7325 | 4.9 |
| 15.26 | 5.8012 | 2 |
| 17.42 | 5.0865 | 31 |
| 18.081 | 4.9021 | 6.1 |
| 19.301 | 4.5949 | 5.9 |
| 19.781 | 4.4845 | 11.4 |
| 20.4 | 4.3497 | 3.9 |
| 21.739 | 4.0847 | 66.7 |
| 23.359 | 3.805 | 0.9 |
| 25.14 | 3.5394 | 2.4 |
| 25.921 | 3.4345 | 5.9 |
| 27.22 | 3.2734 | 2.5 |
| 28.56 | 3.1228 | 15.2 |
| 29.439 | 3.0315 | 1.6 |
| 30.479 | 2.9304 | 100 |
| 32.18 | 2.7793 | 1 |
| 32.8 | 2.7282 | 1 |
| 33.878 | 2.6438 | 1.1 |
| 34.92 | 2.5672 | 18.6 |
| 39.439 | 2.2829 | 2 |
| 41.5 | 2.1741 | 0.7 |
| 44 | 2.0563 | 4.1 |

In some embodiments of the present disclosure, Formula I-Form II is characterized by an XRPD pattern comprising a peak at one of the angles listed in Table 2. In other aspects, Formula I-Form II is characterized by an XRPD pattern comprising more than one peak at one of the angles listed in Table 2 above. In other aspects, Formula I-Form II is characterized by an XRPD pattern comprising two peaks selected from the angles listed in Table 2 above. In other aspects, Formula I-Form II is characterized by an XRPD pattern comprising three peaks selected from the angles listed in Table 2 above. In other aspects, Formula I-Form II is characterized by an XRPD pattern comprising four peaks selected from the angles listed in Table 2 above. In other aspects, Formula I-Form II is characterized by an XRPD pattern comprising five peaks selected from the angles listed in Table 2 above. In other aspects, Formula I-Form II is characterized by an XRPD pattern comprising six peaks selected from the angles listed in Table 2 above. In other aspects, Formula I-Form II is characterized by an XRPD pattern comprising seven peaks selected from the angles listed in Table 2 above. In other aspects, Formula I-Form II is characterized by an XRPD pattern comprising eight peaks selected from the angles listed in Table 2 above. In other aspects, Formula I-Form II is characterized by an XRPD pattern comprising nine peaks selected from the angles listed in Table 2 above. In other aspects, Formula I-Form II is characterized by an XRPD pattern comprising ten peaks selected from the angles listed in Table 2 above. In other aspects, Formula I-Form II is characterized by an XRPD pattern comprising more than ten peaks selected from the angles listed in Table 2 above.

In some embodiments, Formula I-Form II is characterized by an XRPD pattern comprising a peak 9.2, 21.7, and 30.5 degrees ± 0.2 degrees 2-theta. In other embodiments, Formula I-Form II is characterized by an XRPD pattern comprising peaks at 9.2, 12.6, 17.4, and 30.5 degrees ± 0.2 degrees 2-theta. In other embodiments, Formula I-Form II is characterized by an XRPD pattern comprising peaks at 17.4, 18.1, 19.3, 19.8, and 21.7 degrees ± 0.2 degree 2-theta. In other embodiments, Formula I-Form II is characterized by an XRPD pattern comprising peaks at 17.4, 18.1, 19.3, 19.8, and 30.5 degrees ± 0.2 degree 2-theta.. In other embodiments, Formula I-Form II is characterized by an XRPD pattern comprising peaks at 12.6, 17.4, 18.1, 19.3, 19.8, 21.7, 28.6, and 30.5 degrees ± 0.2 degree 2-theta.. In other embodiments, Formula I-Form II is characterized by an XRPD pattern comprising peaks at 9.2, 12.6, 17.4, 18.1, 19.3, 19.8, 21.7, 28.6, 30.5, and 34.9 degrees ± 0.2 degree 2-theta.

In some embodiments of the present disclosure, Formula I-Form II is characterized by an XRPD pattern comprising peaks at two or more of 9.2, 12.6, 17.4, 18.1, 19.3, 19.8, 21.7, 28.6, 30.5, and 34.9 degrees ± 0.2 degrees 2-theta.

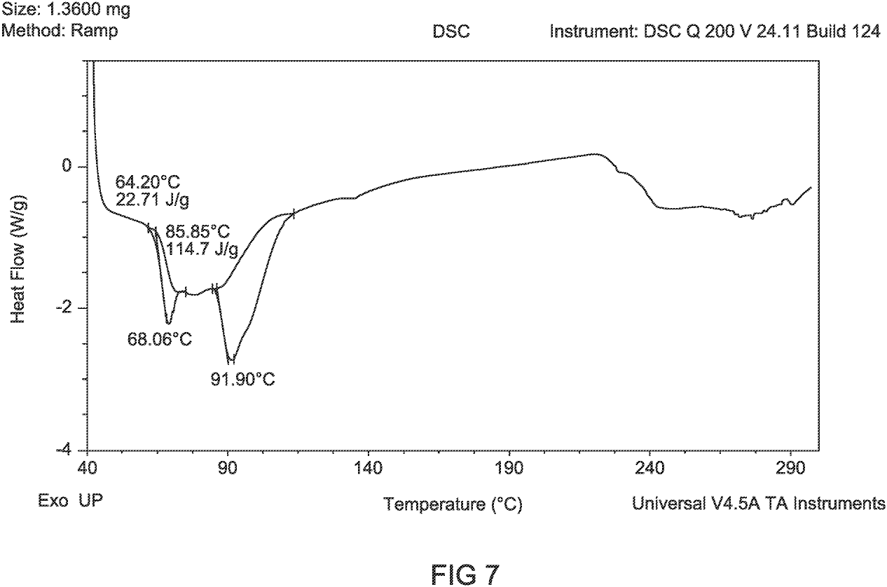

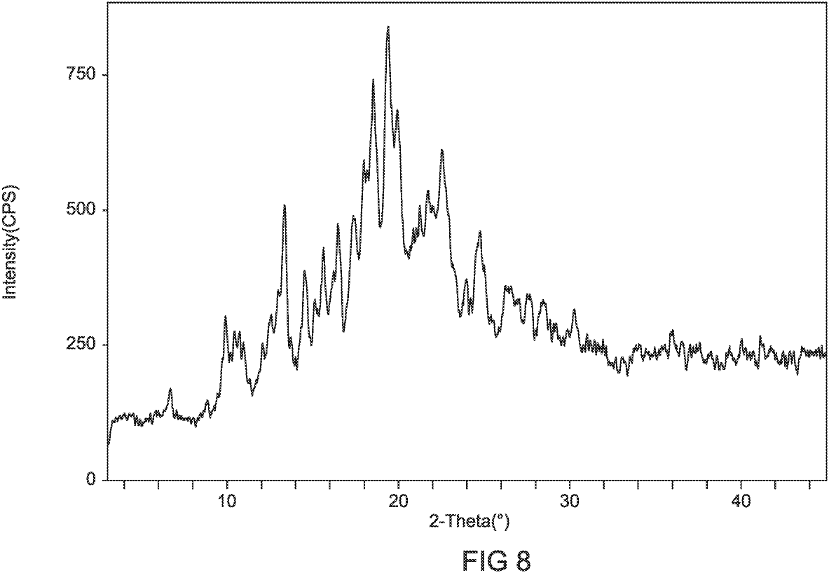

In some embodiments, Formula I-Form II can be characterized by a DSC thermogram substantially as shown in FIG. 7. As FIG. 7 shows, Formula I-Form II produced an endothermic peak at 68.06° C., with a peak onset temperature of 64.20° C., and an enthalpy of melting of 22.71 J/g, followed by produced an endothermic peak at 91.90° C., with a peak onset temperature of 85.85° C., and an enthalpy of melting of 114.7 J/g, when heated at a rate of 10° C./min. In some embodiments of the present disclosure, Formula I-Form II is characterized by a DSC thermogram comprising an endothermic peak at about 68° C. In other embodiments of the present disclosure, Formula I-Form II is characterized by a DSC enthalpy of melting of about 23 J/g. In other embodiments, Formula I-Form II is characterized by a DSC thermogram comprising an endothermic peak at about 92° C. In other embodiments of the present disclosure, Formula I-Form II is characterized by a DSC enthalpy of melting of about 115 J/g.

In some embodiments of the present disclosure, Formula I-Form II is characterized by an XRPD pattern comprising peaks at one or more of 9.2, 12.6, 17.4, 18.1, 19.3, 19.8, 21.7, 28.6, 30.5, and 34.9 degrees ± 0.2 degrees 2-theta, and a DSC thermogram comprising an endothermic peak at about 68° C. when heated at a rate of 10° C./min.

In some embodiments, the disclosure is directed to a choline salt of a compound of Formula I, having the formula IA:

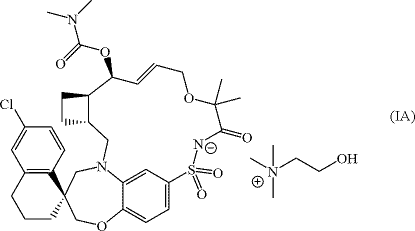

In some embodiments, the disclosure is directed to a crystalline form of the choline salt of the compound of Formula I.

In some embodiments, the choline salt of Formula I is substantially free of any other salt or solid form of Formula I.

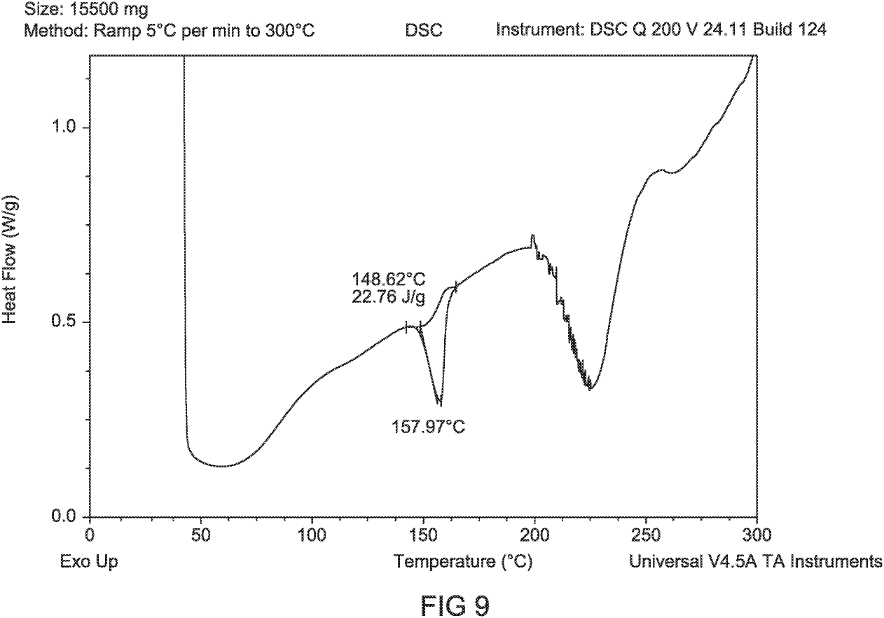

In some embodiments, the choline salt of formula I exhibits an XRPD substantially as shown in FIG. 8. The XRPD of the choline salt of formula I shown in FIG. 8 comprises reflection angles (degrees 2-theta ± 0.2 degrees 2-theta), line spacings (d values), and relative intensities as shown in Table 3:

TABLE 3

XRPD Data for crystalline form of the choline salt of Formula I (Formula IA) shown inFIG. 8 .

| Angle (degrees 2-theta ± 0.2 degrees 2-theta) | d Value (Å) | Relative Intensity |
|---|---|---|
| 6.679 | 13.2224 | 17.1 |
| 9.88 | 8.9447 | 42 |
| 10.94 | 8.0808 | 25.1 |
| 13.321 | 6.6412 | 79.5 |
| 14.5 | 6.1036 | 40.2 |
| 15.619 | 5.6687 | 46.1 |
| 16.46 | 5.3809 | 51.8 |
| 17.359 | 5.1043 | 32.1 |
| 17.979 | 4.9297 | 53.4 |
| 18.52 | 4.7869 | 78.2 |
| 19.399 | 4.5718 | 100 |
| 19.959 | 4.445 | 64.2 |
| 21.758 | 4.0812 | 34.5 |
| 22.618 | 3.928 | 60.1 |
| 23.941 | 3.7138 | 17.6 |
| 24.741 | 3.5956 | 45.6 |
| 26.4 | 3.3733 | 23.8 |
| 27.661 | 3.2223 | 15.3 |
| 28.44 | 3.1358 | 18.9 |
| 30.279 | 2.9494 | 18.1 |
| 36.021 | 2.4913 | 16.6 |
| 41.157 | 2.1915 | 14.2 |

In some embodiments of the present disclosure, the choline salt of Formula I is characterized by an XRPD pattern comprising a peak at one of the angles listed in Table 3. In other aspects, the choline salt of Formula I is characterized by an XRPD pattern comprising more than one peak at one of the angles listed in Table 3 above. In other aspects, the choline salt of Formula I is characterized by an XRPD pattern comprising two peaks selected from the angles listed in Table 3 above. In other aspects, the choline salt of Formula I is characterized by an XRPD pattern comprising three peaks selected from the angles listed in Table 3 above. In other aspects, the choline salt of Formula I is characterized by an XRPD pattern comprising four peaks selected from the angles listed in Table 3 above. In other aspects, the choline salt of Formula I is characterized by an XRPD pattern comprising five peaks selected from the angles listed in Table 3 above. In other aspects, the choline salt of Formula I is characterized by an XRPD pattern comprising six peaks selected from the angles listed in Table 3 above. In other aspects, the choline salt of Formula I is characterized by an XRPD pattern comprising seven peaks selected from the angles listed in Table 3 above. In other aspects, the choline salt of Formula I is characterized by an XRPD pattern comprising eight peaks selected from the angles listed in Table 3 above. In other aspects, the choline salt of Formula I is characterized by an XRPD pattern comprising nine peaks selected from the angles listed in Table 3 above. In other aspects, the choline salt of Formula I is characterized by an XRPD pattern comprising ten peaks selected from the angles listed in Table 3 above. In other aspects, the choline salt of Formula I is characterized by an XRPD pattern comprising more than ten peaks selected from the angles listed in Table 3 above.

In some embodiments, the choline salt of Formula I is characterized by an XRPD pattern comprising peaks at 19.4, and 20.0 degrees ± 0.2 degrees 2-theta. In other embodiments, the choline salt of Formula I is characterized by an XRPD pattern comprising peaks at 18.5, 19.4, 20.0, and 22.6 degrees ± 0.2 degrees 2-theta. In other embodiments, the choline salt of Formula I is characterized by an XRPD pattern comprising peaks at 18.5, 19.4, 20.0, 22.6, and 24.7 degrees ± 0.2 degree 2-theta. In other embodiments, the choline salt of Formula I is characterized by an XRPD pattern comprising peaks at 13.3, 18.5, 19.4, 20.0, and 22.6 degrees ± 0.2 degree 2-theta.. In other embodiments, the choline salt of Formula I is characterized by an XRPD pattern comprising peaks at 13.3, 18.5, 19.4, 20.0, 22.6, and 24.7 degrees ± 0.2 degree 2-theta.. In other embodiments, the choline salt of Formula I is characterized by an XRPD pattern comprising peaks at 9.9, 13.3, 18.5, 19.4, 20.0, 22.6, and 24.7 degrees ± 0.2 degree 2-theta.

In some embodiments of the present disclosure, the choline salt of Formula I is characterized by an XRPD pattern comprising peaks at two or more of 9.9, 13.3, 18.5, 19.4, 20.0, 22.6, and 24.7 degrees ± 0.2 degrees 2-theta.

In some embodiments, the choline salt of Formula I can be characterized by a DSC thermogram substantially as shown in FIG. 9. As FIG. 9 shows, the choline salt of Formula I produced an endothermic peak at 157.97° C., with a peak onset temperature of 148.62° C., and an enthalpy of melting of 22.76 J/g, when heated at a rate of 10° C./min. In some embodiments of the present disclosure, the choline salt of Formula I is characterized by a DSC thermogram comprising an endothermic peak at about 158° C. In other embodiments of the present disclosure, the choline salt of Formula I is characterized by a DSC enthalpy of melting of about 23 J/g.

In some embodiments of the present disclosure, the choline salt of Formula I is characterized by an XRPD pattern comprising peaks at one or more of 9.9, 13.3, 18.5, 19.4, 20.0, 22.6, and 24.7 degrees ± 0.2 degrees 2-theta, and a DSC thermogram comprising an endothermic peak at about 158° C. when heated at a rate of 10° C./min.

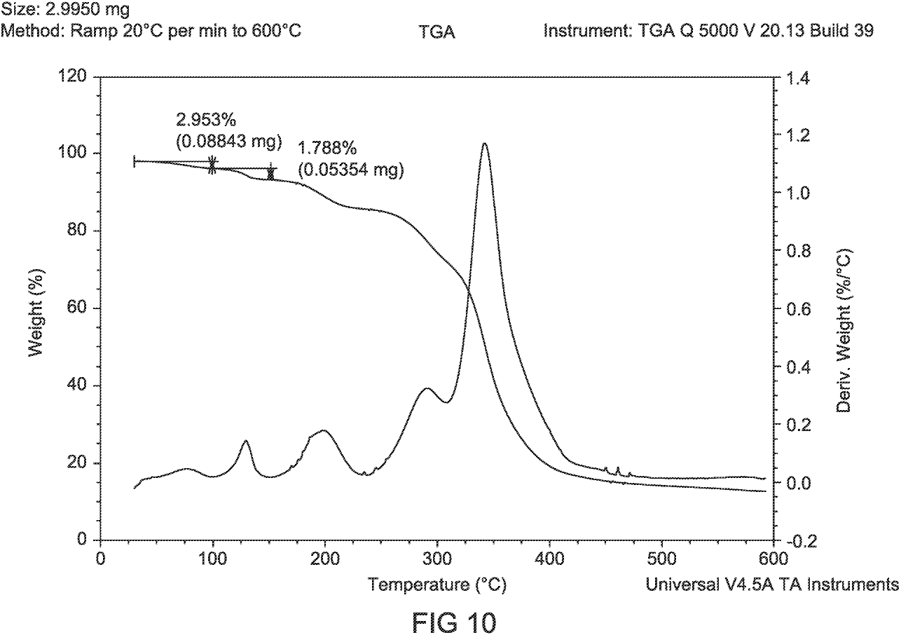

In some embodiments of the present disclosure, the choline salt of Formula I is characterized by an TGA profile substantially as shown in FIG. 10. As shown in FIG. 10, the choline salt of Formula I loses about 4.7% by weight upon heating to 250° C. at 20° C. per minute.

In some embodiments, the disclosure is directed to a benzathine salt of a compound of Formula I, having the formula IB:

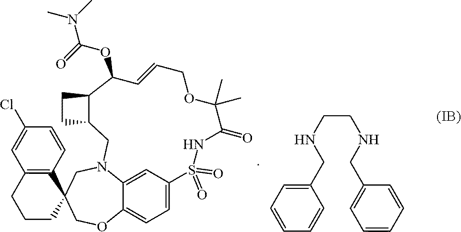

In some embodiments, the disclosure is directed to a crystalline form of the benzathine salt of a compound of Formula I.

In some embodiments, the benzathine salt of Formula I is substantially free of any other salt or solid form of Formula I.

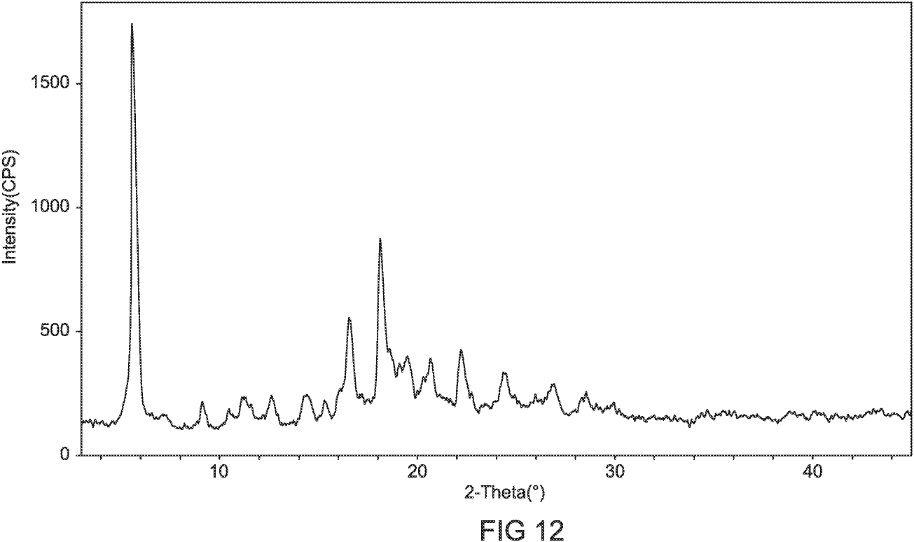

In some embodiments, the benzathine salt of formula I exhibits an XRPD substantially as shown in FIG. 12. The XRPD of the benzathine salt of formula I shown in FIG. 12 comprises reflection angles (degrees 2-theta ± 0.2 degrees 2-theta), line spacings (d values), and relative intensities as shown in Table 4:

TABLE 4

XRPD Data for crystalline form of the benzathine salt of Formula I (Formula IB) shown in FIG. 12 .

| Angle (degrees 2-theta ± 0.2 degrees 2-theta) | d Value (Å) | Relative Intensity |
|---|---|---|
| 5.78 | 15.2777 | 100 |
| 9.158 | 9.6487 | 6.3 |
| 10.497 | 8.4208 | 4.1 |
| 11.319 | 7.811 | 6.5 |
| 12.64 | 6.9976 | 6.4 |
| 14.42 | 6.1373 | 6.8 |
| 15.339 | 5.7717 | 4.6 |
| 16.6 | 5.3359 | 23 |
| 18.22 | 4.865 | 37.5 |
| 19.54 | 4.5392 | 8.4 |
| 20.7 | 4.2875 | 10.8 |
| 22.24 | 3.9938 | 14.6 |
| 24.46 | 3.6362 | 9.2 |
| 26.94 | 3.3069 | 7.2 |
| 28.559 | 3.123 | 5.5 |

In some embodiments of the present disclosure, the benzathine salt of Formula I is characterized by an XRPD pattern comprising a peak at one of the angles listed in Table 4. In other aspects, the benzathine salt of Formula I is characterized by an XRPD pattern comprising more than one peak at one of the angles listed in Table 4 above. In other aspects, the benzathine salt of Formula I is characterized by an XRPD pattern comprising two peaks selected from the angles listed in Table 4 above. In other aspects, the benzathine salt of Formula I is characterized by an XRPD pattern comprising three peaks selected from the angles listed in Table 4 above. In other aspects, the benzathine salt of Formula I is characterized by an XRPD pattern comprising four peaks selected from the angles listed in Table 4 above. In other aspects, the benzathine salt of Formula I is characterized by an XRPD pattern comprising five peaks selected from the angles listed in Table 4 above. In other aspects, the benzathine salt of Formula I is characterized by an XRPD pattern comprising six peaks selected from the angles listed in Table 4 above. In other aspects, the benzathine salt of Formula I is characterized by an XRPD pattern comprising seven peaks selected from the angles listed in Table 4 above. In other aspects, the benzathine salt of Formula I is characterized by an XRPD pattern comprising eight peaks selected from the angles listed in Table 4 above. In other aspects, the benzathine salt of Formula I is characterized by an XRPD pattern comprising nine peaks selected from the angles listed in Table 4 above. In other aspects, the benzathine salt of Formula I is characterized by an XRPD pattern comprising ten peaks selected from the angles listed in Table 4 above. In other aspects, the benzathine salt of Formula I is characterized by an XRPD pattern comprising more than ten peaks selected from the angles listed in Table 4 above.

In some embodiments, the benzathine salt of Formula I is characterized by an XRPD pattern comprising peaks at 5.8, and 18.2 degrees ± 0.2 degrees 2-theta. In other embodiments, the benzathine salt of Formula I is characterized by an XRPD pattern comprising peaks at 5.8, 16.6, and 18.2 degrees ± 0.2 degrees 2-theta. In other embodiments, the benzathine salt of Formula I is characterized by an XRPD pattern comprising peaks at 5.8, 16.6, 18.2, and 20.7 degrees ± 0.2 degree 2-theta. In other embodiments, the benzathine salt of Formula I is characterized by an XRPD pattern comprising peaks at 5.8, 12.6, 16.6, 18.2, and 22.2 degrees ± 0.2 degree 2-theta.. In other embodiments, the benzathine salt of Formula I is characterized by an XRPD pattern comprising peaks at 5.8, 12.6, 16.6, 18.2, and 20.7 degrees ± 0.2 degree 2-theta.. In other embodiments, the benzathine salt of Formula I is characterized by an XRPD pattern comprising peaks at 5.8, 12.6, 16.6, 18.2, 20.7, and 22.2 degrees ± 0.2 degree 2-theta.

In some embodiments of the present disclosure, the benzathine salt of Formula I is characterized by an XRPD pattern comprising peaks at two or more of 5.8, 12.6, 16.6, 18.2, 20.7, and 22.2 degrees ± 0.2 degrees 2-theta.

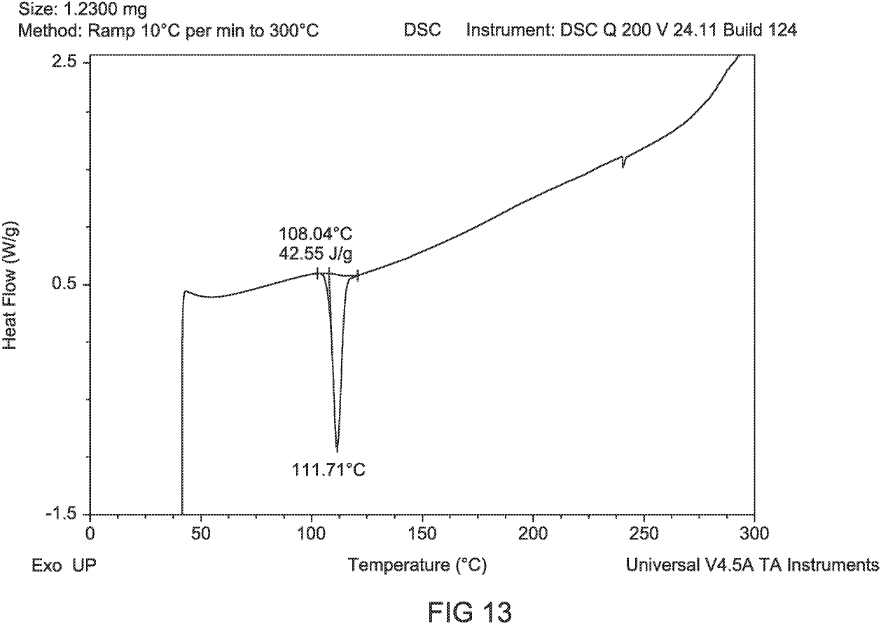

In some embodiments, the benzathine salt of Formula I can be characterized by a DSC thermogram substantially as shown in FIG. 13. As FIG. 13 shows, the benzathine salt of Formula I produced an endothermic peak at 111.71° C., with a peak onset temperature of 108.04° C., and an enthalpy of melting of 42.55 J/g, when heated at a rate of 10° C./min. In some embodiments of the present disclosure, the benzathine salt of Formula I is characterized by a DSC thermogram comprising an endothermic peak at about 112° C. In other embodiments of the present disclosure, the benzathine salt of Formula I is characterized by a DSC enthalpy of melting of about 43 J/g.

In some embodiments of the present disclosure, the benzathine salt of Formula I is characterized by an XRPD pattern comprising peaks at one or more of 5.8, 12.6, 16.6, 18.2, 20.7, and 22.2 degrees ± 0.2 degrees 2-theta, and a DSC thermogram comprising an endothermic peak at about 112° C. when heated at a rate of 10° C./min.

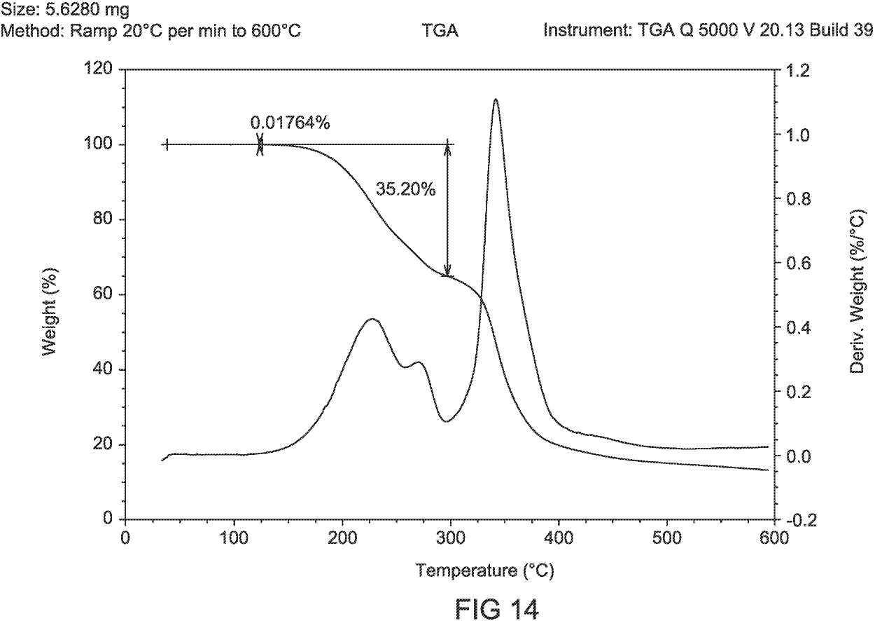

In some embodiments of the present disclosure, the benzathine salt of Formula I is characterized by an TGA profile substantially as shown in FIG. 14. As shown in FIG. 14, the benzathine salt of Formula I loses about 35.2% by weight upon heating to 300° C. at 20° C. per minute.

In some embodiments, the disclosure is directed to an imidazole salt of a compound of Formula I, having formula IC:

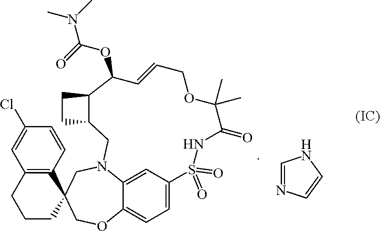

In some embodiments, the disclosure is directed to a crystalline form of the imidazole salt of a compound of Formula I.

In some embodiments, the imidazole salt of Formula I is substantially free of any other salt or solid form of Formula I.

In some embodiments, the imidazole salt of Formula I exhibits an XRPD substantially as shown in FIG. 16. The XRPD of the imidazole salt of formula I shown in FIG. 16 comprises reflection angles (degrees 2-theta ± 0.2 degrees 2-theta), line spacings (d values), and relative intensities as shown in Table 5:

TABLE 5

XRPD Data for crystalline form of the imidazole salt of Formula I (Formula IC) shown in FIG. 16 .

| Angle (degrees 2-theta ± 0.2 degrees 2-theta) | d Value (Å) | Relative Intensity |
|---|---|---|
| 6.481 | 13.6275 | 44.7 |
| 6.98 | 12.6533 | 47.5 |
| 8.756 | 10.0902 | 9.7 |
| 9.72 | 9.0924 | 20.2 |
| 12.381 | 7.1433 | 24.3 |
| 13.36 | 6.6219 | 19.8 |
| 14.059 | 6.294 | 77.8 |
| 16.96 | 5.2236 | 100 |
| 17.921 | 4.9456 | 56.6 |
| 18.76 | 4.7263 | 47.3 |
| 19.861 | 4.4666 | 30.1 |
| 20.56 | 4.3163 | 38.9 |
| 22.02 | 4.0333 | 14.8 |
| 22.86 | 3.8869 | 44.7 |
| 23.779 | 3.7387 | 48.2 |
| 24.441 | 3.639 | 27.5 |
| 26.461 | 3.3656 | 34 |
| 28.58 | 3.1207 | 20.4 |
| 29.277 | 3.048 | 18.1 |
| 30.4 | 2.9379 | 18.5 |
| 39.917 | 2.2566 | 13.1 |

In some embodiments of the present disclosure, the imidazole salt of Formula I is characterized by an XRPD pattern comprising a peak at one of the angles listed in Table 5. In other aspects, the imidazole salt of Formula I is characterized by an XRPD pattern comprising more than one peak at one of the angles listed in Table 5 above. In other aspects, the imidazole salt of Formula I is characterized by an XRPD pattern comprising two peaks selected from the angles listed in Table 5 above. In other aspects, the imidazole salt of Formula I is characterized by an XRPD pattern comprising three peaks selected from the angles listed in Table 5 above. In other aspects, the imidazole salt of Formula I is characterized by an XRPD pattern comprising four peaks selected from the angles listed in Table 5 above. In other aspects, the imidazole salt of Formula I is characterized by an XRPD pattern comprising five peaks selected from the angles listed in Table 5 above. In other aspects, the imidazole salt of Formula I is characterized by an XRPD pattern comprising six peaks selected from the angles listed in Table 5 above. In other aspects, the imidazole salt of Formula I is characterized by an XRPD pattern comprising seven peaks selected from the angles listed in Table 5 above. In other aspects, the imidazole salt of Formula I is characterized by an XRPD pattern comprising eight peaks selected from the angles listed in Table 5 above. In other aspects, the imidazole salt of Formula I is characterized by an XRPD pattern comprising nine peaks selected from the angles listed in Table 5 above. In other aspects, the imidazole salt of Formula I is characterized by an XRPD pattern comprising ten peaks selected from the angles listed in Table 5 above. In other aspects, the imidazole salt of Formula I is characterized by an XRPD pattern comprising more than ten peaks selected from the angles listed in Table 5 above.

In some embodiments, the imidazole salt of Formula I is characterized by an XRPD pattern comprising peaks at 14.1, and 17.0 degrees ± 0.2 degrees 2-theta. In other embodiments, the imidazole salt of Formula I is characterized by an XRPD pattern comprising peaks at 14.1, 17.0, 17.9, 18.8, and 20.6 degrees ± 0.2 degrees 2-theta. In other embodiments, the imidazole salt of Formula I is characterized by an XRPD pattern comprising peaks at 14.1, 17.0, 17.9, 18.8, 20.6, 22.0, 22.9, and 23.8, degrees ± 0.2 degree 2-theta. In other embodiments, the imidazole salt of Formula I is characterized by an XRPD pattern comprising peaks at 6.5, 7.0, 14.1, 17.0, 17.9, 18.8, 20.6, 22.0, 22.9, and 23.8 degrees ± 0.2 degree 2-theta.. In other embodiments, the imidazole salt of Formula I is characterized by an XRPD pattern comprising peaks at 14.1, 17.0, 17.9, 18.8, 20.6, 22.0, 22.9, 23.8, 24.4, and 26.5 degrees ± 0.2 degree 2-theta.. In other embodiments, the imidazole salt of Formula I is characterized by an XRPD pattern comprising peaks at 6.5, 7.0, 14.1, 17.0, 17.9, 18.8, 20.6, 22.0, 22.9, 23.8, 24.4, and 26.5 degrees ± 0.2 degree 2-theta.

In some embodiments of the present disclosure, the imidazole salt of Formula I is characterized by an XRPD pattern comprising peaks at two or more of 6.5, 7.0, 14.1, 17.0, 17.9, 18.8, 20.6, 22.0, 22.9, 23.8, 24.4, and 26.5 degrees ± 0.2 degrees 2-theta.

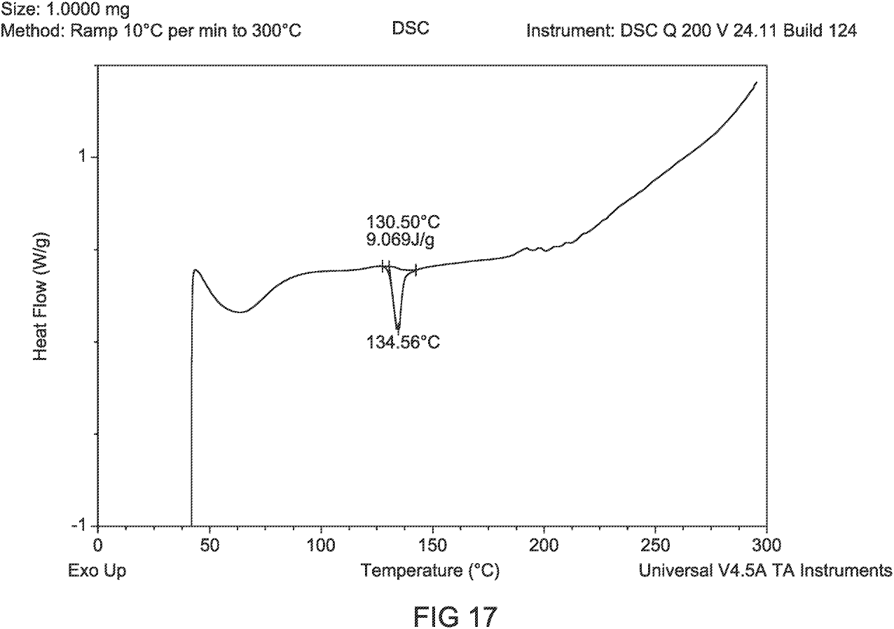

In some embodiments, the imidazole salt of Formula I can be characterized by a DSC thermogram substantially as shown in FIG. 17. As FIG. 17 shows, the imidazole salt of Formula I produced an endothermic peak at 134.56° C., with a peak onset temperature of 130.50° C., and an enthalpy of melting of 9.069 J/g, when heated at a rate of 10° C./min. In some embodiments of the present disclosure, the imidazole salt of Formula I is characterized by a DSC thermogram comprising an endothermic peak at about 135° C. In other embodiments of the present disclosure, the imidazole salt of Formula I is characterized by a DSC enthalpy of melting of about 9.1 J/g.

In some embodiments of the present disclosure, the imidazole salt of Formula I is characterized by an XRPD pattern comprising peaks at one or more of 6.5, 7.0, 14.1, 17.0, 17.9, 18.8, 20.6, 22.0, 22.9, 23.8, 24.4, and 26.5 degrees ± 0.2 degrees 2-theta, and a DSC thermogram comprising an endothermic peak at about 135° C. when heated at a rate of 10° C./min.

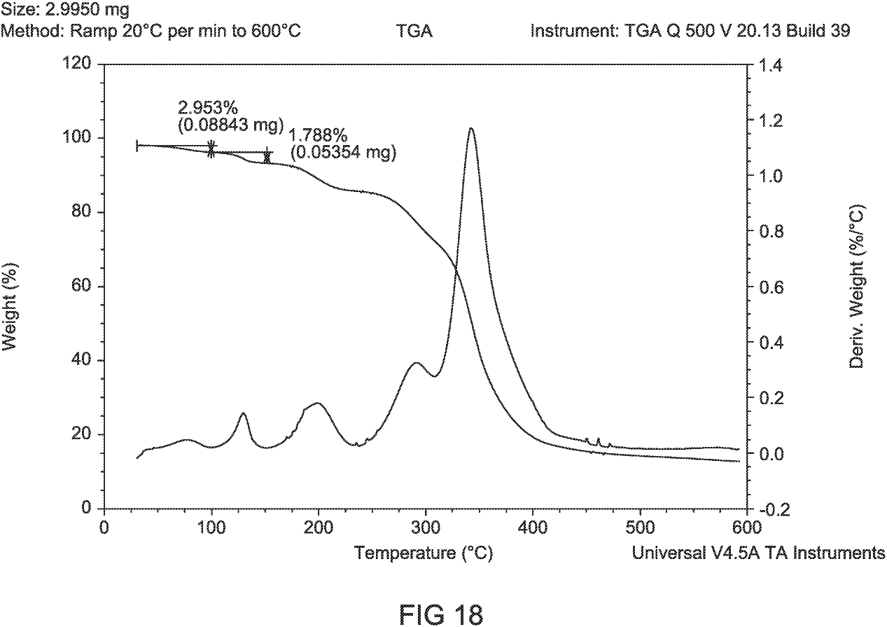

In some embodiments of the present disclosure, the imidazole salt of Formula I is characterized by an TGA profile substantially as shown in FIG. 18. As shown in FIG. 18, the imidazole salt of Formula I loses about 4.7% by weight upon heating to 200° C. at 20° C. per minute.

In some embodiments, the disclosure is directed to a piperazine salt of a compound of Formula I, having the formula ID:

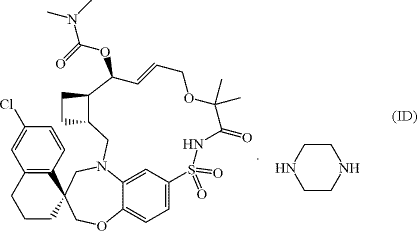

In some embodiments, the disclosure is directed to a crystalline form of the piperazine salt of formula I.

In some embodiments, the piperazine salt of Formula I is substantially free of any other salt or solid form of Formula I.

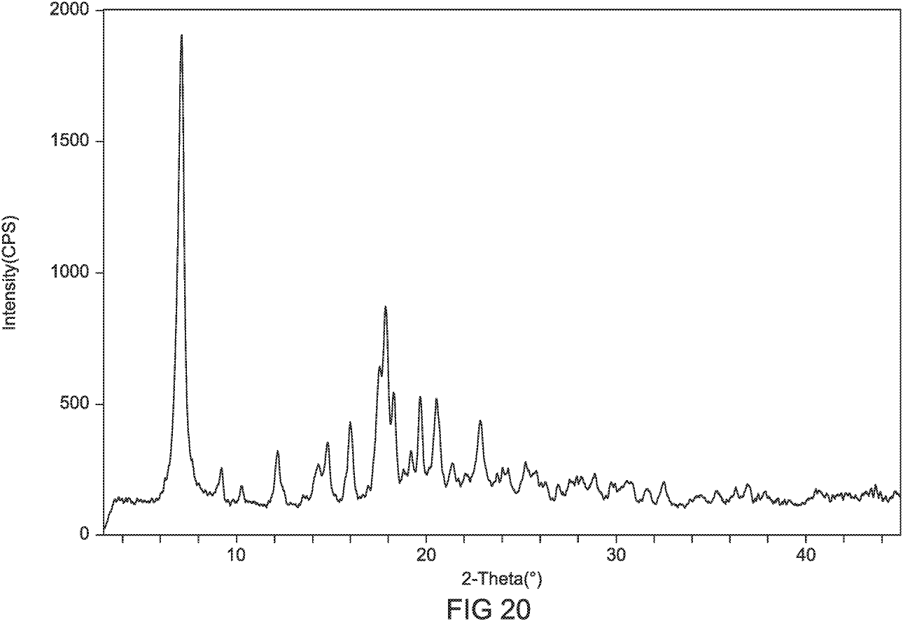

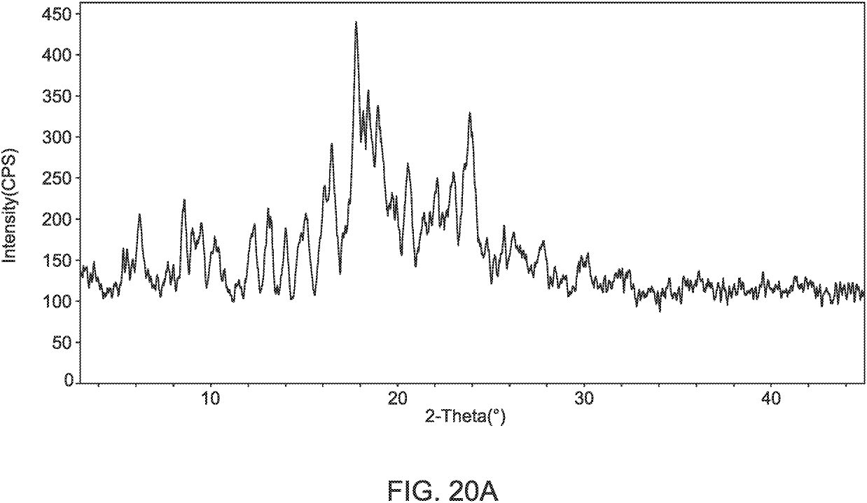

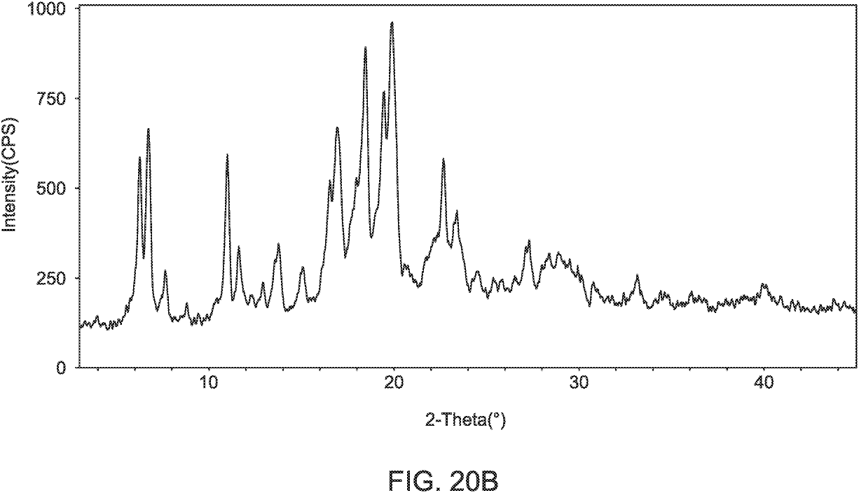

In some embodiments, the piperazine salt of formula I (Form 1) exhibits an XRPD substantially as shown in FIG. 20. The XRPD of the piperazine salt of formula I shown in FIG. 20 comprises reflection angles (degrees 2-theta ± 0.2 degrees 2-theta), line spacings (d values), and relative intensities as shown in Table 6:

TABLE 6

XRPD Data for crystalline form of the piperazine salt of Formula I (Formula ID-Form 1) shown in FIG. 20 .

| Angle (degrees 2-theta ± 0.2 degrees 2-theta) | d Value (Å) | Relative Intensity |
|---|---|---|
| 7.12 | 12.4059 | 100 |
| 9.219 | 9.5845 | 7.2 |
| 10.334 | 8.5528 | 3.4 |
| 12.18 | 7.2607 | 12.2 |
| 14.3 | 6.1884 | 8.6 |
| 14.819 | 5.9728 | 12.9 |
| 16 | 5.5346 | 16.7 |
| 17.86 | 4.9622 | 39.2 |
| 19.182 | 4.6231 | 6 |
| 19.681 | 4.5071 | 18.8 |
| 20.54 | 4.3204 | 18.3 |
| 21.38 | 4.1525 | 5 |
| 22.841 | 3.8902 | 14.1 |
| 24.32 | 3.6568 | 4.8 |
| 25.24 | 3.5256 | 7.1 |
| 26.959 | 3.3045 | 3 |
| 27.68 | 3.22 | 3.5 |
| 28.161 | 3.1662 | 5 |
| 28.899 | 3.0869 | 4.7 |
| 29.94 | 2.982 | 3.6 |
| 30.761 | 2.9043 | 4.2 |
| 31.679 | 2.8222 | 3.1 |
| 32.54 | 2.7494 | 5 |
| 35.357 | 2.5365 | 2.5 |
| 36.319 | 2.4715 | 3.3 |
| 36.92 | 2.4326 | 4.1 |
| 40.681 | 2.216 | 2.7 |

In some embodiments of the present disclosure, the piperazine salt of Formula 1 (Form 1) (Form 1) is characterized by an XRPD pattern comprising a peak at one of the angles listed in Table 6. In other aspects, the piperazine salt of Formula 1 (Form 1) is characterized by an XRPD pattern comprising more than one peak at one of the angles listed in Table 6 above. In other aspects, the piperazine salt of Formula 1 (Form 1) is characterized by an XRPD pattern comprising two peaks selected from the angles listed in Table 6 above. In other aspects, the piperazine salt of Formula 1 (Form 1) is characterized by an XRPD pattern comprising three peaks selected from the angles listed in Table 6 above. In other aspects, the piperazine salt of Formula 1 (Form 1) is characterized by an XRPD pattern comprising four peaks selected from the angles listed in Table 6 above. In other aspects, the piperazine salt of Formula 1 (Form 1) is characterized by an XRPD pattern comprising five peaks selected from the angles listed in Table 6 above. In other aspects, the piperazine salt of Formula 1 (Form 1) is characterized by an XRPD pattern comprising six peaks selected from the angles listed in Table 6 above. In other aspects, the piperazine salt of Formula 1 (Form 1) is characterized by an XRPD pattern comprising seven peaks selected from the angles listed in Table 6 above. In other aspects, the piperazine salt of Formula 1 (Form 1) is characterized by an XRPD pattern comprising eight peaks selected from the angles listed in Table 6 above. In other aspects, the piperazine salt of Formula 1 (Form 1) is characterized by an XRPD pattern comprising nine peaks selected from the angles listed in Table 6 above. In other aspects, the piperazine salt of Formula 1 (Form 1) is characterized by an XRPD pattern comprising ten peaks selected from the angles listed in Table 6 above. In other aspects, the piperazine salt of Formula 1 (Form 1) is characterized by an XRPD pattern comprising more than ten peaks selected from the angles listed in Table 6 above.

In some embodiments, the piperazine salt of Formula 1 (Form 1) is characterized by an XRPD pattern comprising peaks at 7.1, 12.2, and 14.8 degrees ± 0.2 degrees 2-theta. In other embodiments, the piperazine salt of Formula 1 (Form 1) is characterized by an XRPD pattern comprising peaks at 7.1, 12.2, 14.8, and 16.0 degrees ± 0.2 degrees 2-theta. In other embodiments, the piperazine salt of Formula 1 (Form 1) is characterized by an XRPD pattern comprising peaks at 7.1, 12.2, 14.8, 16.0, and 17.9 degrees ± 0.2 degree 2-theta. In other embodiments, the piperazine salt of Formula 1 (Form 1) is characterized by an XRPD pattern comprising peaks at 7.1, 12.2, 14.8, 16.0, 17.9, and 19.7 degrees ± 0.2 degree 2-theta.. In other embodiments, the piperazine salt of Formula 1 (Form 1) is characterized by an XRPD pattern comprising peaks at 7.1, 12.2, 14.8, 16.0, 17.9, 19.7, and 20.5 degrees ± 0.2 degree 2-theta.. In other embodiments, the piperazine salt of Formula 1 (Form 1) is characterized by an XRPD pattern comprising peaks at 7.1, 12.2, 14.8, 16.0, 17.9, 19.7, 20.5, and 22.8 degrees ± 0.2 degree 2-theta.

In some embodiments of the present disclosure, the piperazine salt of Formula 1 (Form 1) is characterized by an XRPD pattern comprising peaks at two or more of 7.1, 12.2, 14.8, 16.0, 17.9, 19.7, 20.5, and 22.8 degrees ± 0.2 degrees 2-theta.

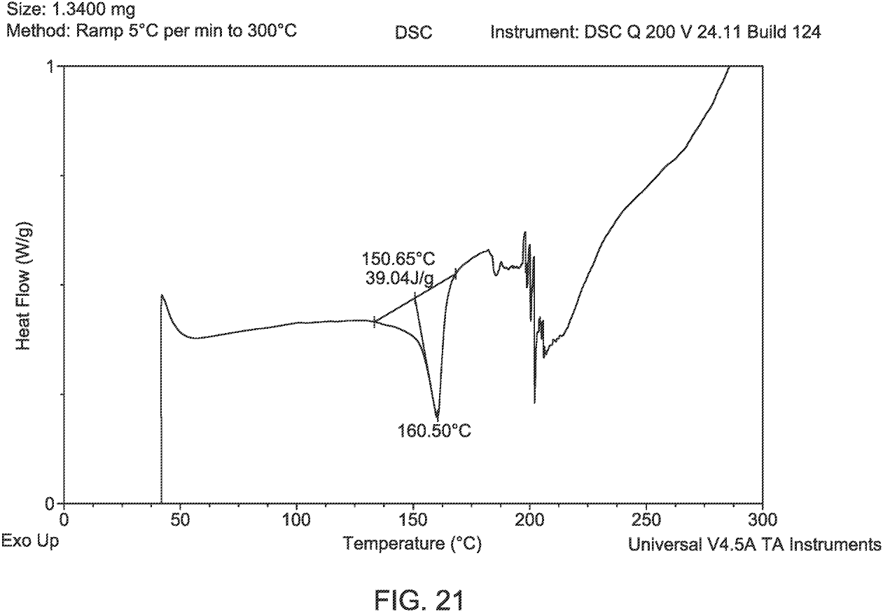

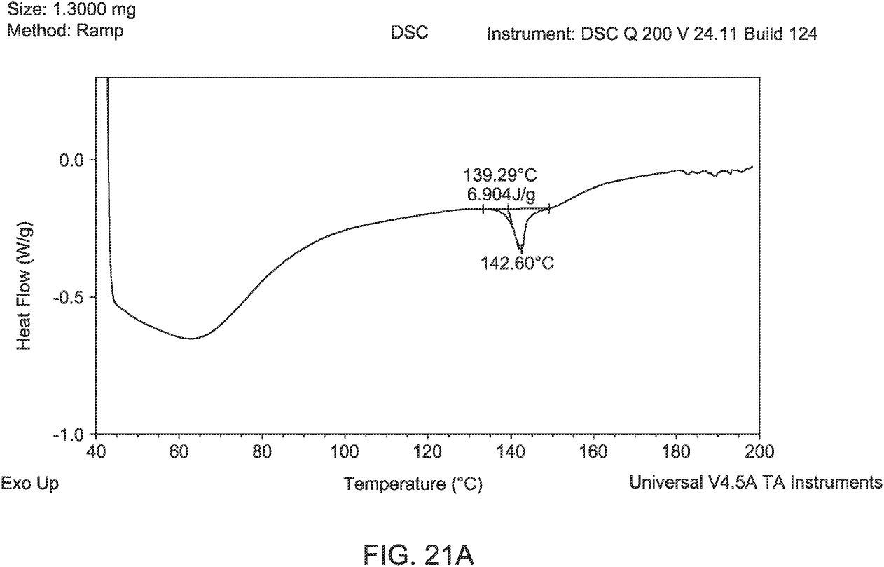

In some embodiments, the piperazine salt of Formula 1 (Form 1) can be characterized by a DSC thermogram substantially as shown in FIG. 21. As FIG. 21 shows, the piperazine salt of Formula 1 (Form 1) produced an endothermic peak at 160.50° C., with a peak onset temperature of 150.65° C., and an enthalpy of melting of 39.04 J/g, when heated at a rate of 10° C./min. In some embodiments of the present disclosure, the piperazine salt of Formula 1 (Form 1) is characterized by a DSC thermogram comprising an endothermic peak at about 160° C. In other embodiments of the present disclosure, the piperazine salt of Formula 1 (Form 1) is characterized by a DSC enthalpy of melting of about 39 J/g.

In some embodiments of the present disclosure, the piperazine salt of Formula 1 (Form 1) is characterized by an XRPD pattern comprising peaks at one or more of 7.1, 12.2, 14.8, 16.0, 17.9, 19.7, 20.5, and 22.8 degrees ± 0.2 degrees 2-theta, and a DSC thermogram comprising an endothermic peak at about 160° C. when heated at a rate of 10° C./min.

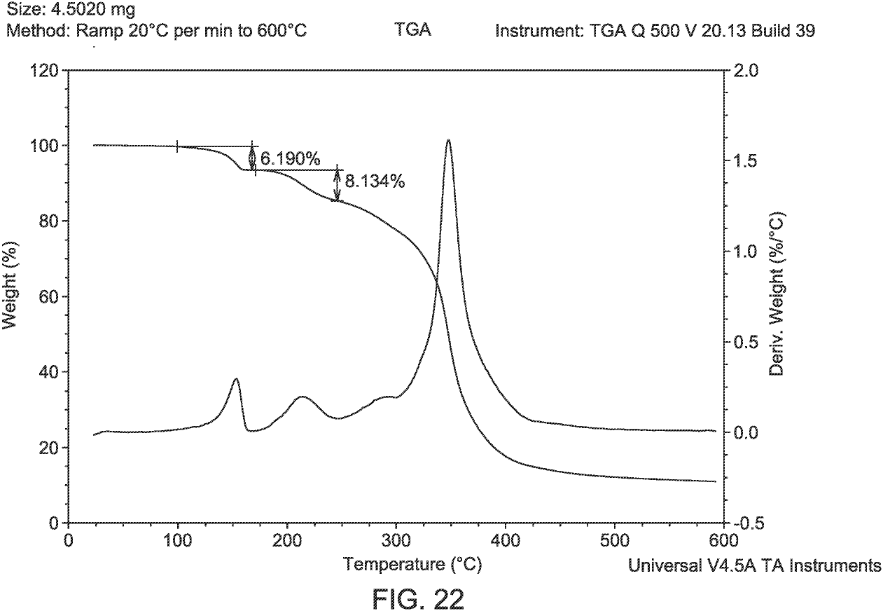

In some embodiments of the present disclosure, the piperazine salt of Formula 1 (Form 1) is characterized by an TGA profile substantially as shown in FIG. 22. As shown in FIG. 22, the piperazine salt of Formula 1 (Form 1) loses about 14.3% by weight upon heating to 300° C. at 20° C. per minute.

In some embodiments, the piperazine salt of Formula I (Form 2) exhibits an XRPD substantially as shown in FIG. 20A. The XRPD of the piperazine salt of Formula I (Form 2) shown in FIG. 20A comprises reflection angles (degrees 2-theta ± 0.2 degrees 2-theta), line spacings (d values), and relative intensities as shown in Table 6A:

TABLE 6A

XRPD Data for crystalline form of the piperazine salt of Formula I (Formula ID-Form 2) shown in FIG. 20A.

| Angle (degrees 2-theta ± 0.2 degrees 2-theta) | d Value (Å) | Relative Intensity |
|---|---|---|
| 5.503 | 16.0461 | 20.5 |
| 6.218 | 14.2015 | 36.4 |
| 8.58 | 10.2973 | 42.4 |
| 9.462 | 9.3395 | 31.1 |
| 10.202 | 8.6638 | 25 |
| 12.34 | 7.1669 | 34.1 |
| 13.079 | 6.7633 | 41.7 |
| 14.001 | 6.3201 | 31.4 |
| 15.099 | 5.8627 | 37.1 |
| 16.1 | 5.5007 | 46.2 |
| 16.48 | 5.3746 | 62.5 |
| 17.78 | 4.9844 | 100 |
| 18.439 | 4.8077 | 70.8 |
| 19.059 | 4.6527 | 41.7 |
| 20.542 | 4.3201 | 43.6 |
| 22.139 | 4.0119 | 34.1 |
| 22.981 | 3.8667 | 32.2 |
| 23.939 | 3.7142 | 58.3 |
| 25.702 | 3.4632 | 24.2 |
| 26.4 | 3.3733 | 15.2 |
| 27.781 | 3.2086 | 18.2 |
| 30.199 | 2.957 | 18.6 |
| 32.438 | 2.7578 | 14 |
| 36.139 | 2.4834 | 14.4 |

In some embodiments of the present disclosure, the piperazine salt of Formula I (Form 2) is characterized by an XRPD pattern comprising a peak at one of the angles listed in Table 6A. In other aspects, the piperazine salt of Formula I (Form 2) is characterized by an XRPD pattern comprising more than one peak at one of the angles listed in Table 6A above. In other aspects, the piperazine salt of Formula I (Form 2) is characterized by an XRPD pattern comprising two peaks selected from the angles listed in Table 6A above. In other aspects, the piperazine salt of Formula I (Form 2) is characterized by an XRPD pattern comprising three peaks selected from the angles listed in Table 6A above. In other aspects, the piperazine salt of Formula I (Form 2) is characterized by an XRPD pattern comprising four peaks selected from the angles listed in Table 6A above. In other aspects, the piperazine salt of Formula I (Form 2) is characterized by an XRPD pattern comprising five peaks selected from the angles listed in Table 6A above. In other aspects, the piperazine salt of Formula I (Form 2) is characterized by an XRPD pattern comprising six peaks selected from the angles listed in Table 6A above. In other aspects, the piperazine salt of Formula I (Form 2) is characterized by an XRPD pattern comprising seven peaks selected from the angles listed in Table 6A above. In other aspects, the piperazine salt of Formula I (Form 2) is characterized by an XRPD pattern comprising eight peaks selected from the angles listed in Table 6A above. In other aspects, the piperazine salt of Formula I (Form 2) is characterized by an XRPD pattern comprising nine peaks selected from the angles listed in Table 6A above. In other aspects, the piperazine salt of Formula I (Form 2) is characterized by an XRPD pattern comprising ten peaks selected from the angles listed in Table 6A above. In other aspects, the piperazine salt of Formula I (Form 2) is characterized by an XRPD pattern comprising more than ten peaks selected from the angles listed in Table 6A above.

In some embodiments, the piperazine salt of Formula I (Form 2) is characterized by an XRPD pattern comprising peaks at 16.5, and 17.8 degrees ± 0.2 degrees 2-theta. In other embodiments, the piperazine salt of Formula I (Form 2) is characterized by an XRPD pattern comprising peaks at 5.5, 6.2, 8.6, 14.0, 16.5, and 17.8, degrees ± 0.2 degrees 2-theta. In other embodiments, the piperazine salt of Formula I (Form 2) is characterized by an XRPD pattern comprising peaks at 16.5, 17.8, 19.1, 20.5, 22.1, and 23.0 degrees ± 0.2 degree 2-theta. In other embodiments, the piperazine salt of Formula I (Form 2) is characterized by an XRPD pattern comprising peaks at 5.5, 6.2, 8.6, 14.0, 16.5, 17.8, 19.1, and 20.5 degrees ± 0.2 degree 2-theta.. In other embodiments, the piperazine salt of Formula I (Form 2) is characterized by an XRPD pattern comprising peaks at 8.6, 14.0, 16.5, 17.8, 19.1, 20.5, 22.1, and 23.0 degrees ± 0.2 degree 2-theta.. In other embodiments, the piperazine salt of Formula I (Form 2) is characterized by an XRPD pattern comprising peaks at 5.5, 6.2, 8.6, 14.0, 16.5, 17.8, 19.1, 20.5, 22.1, and 23.0 degrees ± 0.2 degree 2-theta.

In some embodiments of the present disclosure, the piperazine salt of Formula I (Form 2) is characterized by an XRPD pattern comprising peaks at two or more of 5.5, 6.2, 8.6, 14.0, 16.5, 17.8, 19.1, 20.5, 22.1, and 23.0 degrees ± 0.2 degrees 2-theta.

In some embodiments, the piperazine salt of Formula I (Form 2) can be characterized by a DSC thermogram substantially as shown in FIG. 21A. As FIG. 21A shows, the piperazine salt of Formula I (Form 2) produced an endothermic peak at 142.60° C., with a peak onset temperature of 139.29° C., and an enthalpy of melting of 6.904 J/g, when heated at a rate of 10° C./min. In some embodiments of the present disclosure, the piperazine salt of Formula I (Form 2) is characterized by a DSC thermogram comprising an endothermic peak at about 143° C. In other embodiments of the present disclosure, the piperazine salt of Formula I (Form 2) is characterized by a DSC enthalpy of melting of about 6.9 J/g.

In some embodiments of the present disclosure, the piperazine salt of Formula I (Form 2) is characterized by an XRPD pattern comprising peaks at one or more of 5.5, 6.2, 8.6, 14.0, 16.5, 17.8, 19.1, 20.5, 22.1, and 23.0 degrees ± 0.2 degrees 2-theta, and a DSC thermogram comprising an endothermic peak at about 143° C. when heated at a rate of 10° C./min.

In some embodiments, the piperazine salt of Formula I (Form 3) exhibits an XRPD substantially as shown in FIG. 20B. The XRPD of the piperazine salt of Formula I (Form 3) shown in FIG. 20B comprises reflection angles (degrees 2-theta ± 0.2 degrees 2-theta), line spacings (d values), and relative intensities as shown in Table 6B:

TABLE 6B

XRPD Data for crystalline form of the piperazine salt of Formula I (Formula ID-Form 3) shown in FIG. 20B.

| Angle (degrees 2-theta ± 0.2 degrees 2-theta) | d Value (Å) | Relative Intensity |
|---|---|---|
| 6.26 | 14.1068 | 67.5 |
| 6.739 | 13.1049 | 81.4 |
| 7.641 | 11.5605 | 18.1 |
| 8.801 | 10.0397 | 8.9 |
| 11 | 8.0367 | 68.1 |
| 11.638 | 7.5974 | 25.5 |
| 12.92 | 6.8461 | 10.7 |
| 13.761 | 6.4299 | 27.5 |
| 15.061 | 5.8775 | 15.6 |
| 16.502 | 5.3674 | 36.9 |
| 16.92 | 5.2356 | 59.1 |
| 18.46 | 4.8023 | 81.7 |
| 19.441 | 4.5622 | 66.2 |
| 19.92 | 4.4536 | 100 |
| 22.68 | 3.9173 | 55 |
| 23.419 | 3.7954 | 26.9 |
| 24.501 | 3.6302 | 9.5 |
| 27.319 | 3.2618 | 20.8 |
| 28.381 | 3.1421 | 13 |
| 28.98 | 3.0785 | 16.6 |
| 31.001 | 2.8823 | 6.9 |
| 33.161 | 2.6993 | 13.4 |
| 40.12 | 2.2457 | 8.8 |

In some embodiments of the present disclosure, the piperazine salt of Formula I (Form 3) is characterized by an XRPD pattern comprising a peak at one of the angles listed in Table 6B. In other aspects, the piperazine salt of Formula I (Form 3) is characterized by an XRPD pattern comprising more than one peak at one of the angles listed in Table 6B above. In other aspects, the piperazine salt of Formula I (Form 3) is characterized by an XRPD pattern comprising two peaks selected from the angles listed in Table 6B above. In other aspects, the piperazine salt of Formula I (Form 3) is characterized by an XRPD pattern comprising three peaks selected from the angles listed in Table 6B above. In other aspects, the piperazine salt of Formula I (Form 3) is characterized by an XRPD pattern comprising four peaks selected from the angles listed in Table 6B above. In other aspects, the piperazine salt of Formula I (Form 3) is characterized by an XRPD pattern comprising five peaks selected from the angles listed in Table 6B above. In other aspects, the piperazine salt of Formula I (Form 3) is characterized by an XRPD pattern comprising six peaks selected from the angles listed in Table 6B above. In other aspects, the piperazine salt of Formula I (Form 3) is characterized by an XRPD pattern comprising seven peaks selected from the angles listed in Table 6B above. In other aspects, the piperazine salt of Formula I (Form 3) is characterized by an XRPD pattern comprising eight peaks selected from the angles listed in Table 6B above. In other aspects, the piperazine salt of Formula I (Form 3) is characterized by an XRPD pattern comprising nine peaks selected from the angles listed in Table 6B above. In other aspects, the piperazine salt of Formula I (Form 3) is characterized by an XRPD pattern comprising ten peaks selected from the angles listed in Table 6B above. In other aspects, the piperazine salt of Formula I (Form 3) is characterized by an XRPD pattern comprising more than ten peaks selected from the angles listed in Table 6B above.

In some embodiments, the piperazine salt of Formula I (Form 3) is characterized by an XRPD pattern comprising peaks at 18.5, 19.4, and 19.9 degrees ± 0.2 degrees 2-theta. In other embodiments, the piperazine salt of Formula I (Form 3) is characterized by an XRPD patte comprising peaks at 16.5, 16.9, 18.5, 19.4, 19.9, and 22.7 degrees ± 0.2 degrees 2-theta. In other embodiments, the piperazine salt of Formula I (Form 3) is characterized by an XRPD pattern comprising peaks at 13.8, 16.5, 16.9, 18.5, 19.4, 19.9, and 22.7 degrees ± 0.2 degree 2-theta. In other embodiments, the piperazine salt of Formula I (Form 3) is characterized by an XRPD pattern comprising peaks at 11.6, 13.8, 16.5, 16.9, 18.5, 19.4, and 19.9 degrees ± 0.2 degree 2-theta.. In other embodiments, the piperazine salt of Formula I (Form 3) is characterized by an XRPD pattern comprising peaks at 11.6, 13.8, 16.5, 16.9, 18.5, 19.4, 19.9, and 22.7 degrees ± 0.2 degree 2-theta.. In other embodiments, the piperazine salt of Formula I (Form 3) is characterized by an XRPD pattern comprising peaks at 6.3, 6.7, 11.0, 11.6, 13.8, 16.5, 16.9, 18.5, 19.4, 19.9, and 22.7 degrees ± 0.2 degree 2-theta.

In some embodiments of the present disclosure, the piperazine salt of Formula I (Form 3) is characterized by an XRPD pattern comprising peaks at two or more of 6.3, 6.7, 11.0, 11.6, 13.8, 16.5, 16.9, 18.5, 19.4, 19.9, and 22.7 degrees ± 0.2 degrees 2-theta.

In some embodiments, the disclosure is directed to a piperidine salt of a compound of Formula I, having the formula IE:

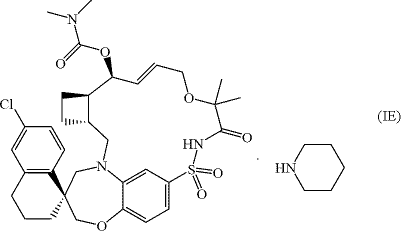

In some embodiments, the disclosure is directed to a crystalline form of the piperidine salt of formula I.

In some embodiments, the piperidine salt of Formula I is substantially free of any other salt or solid form of Formula I.

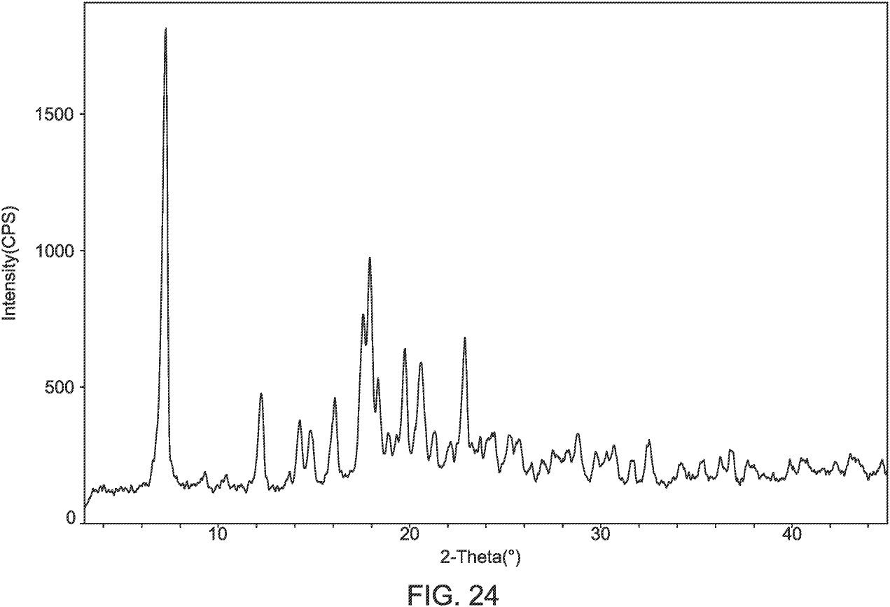

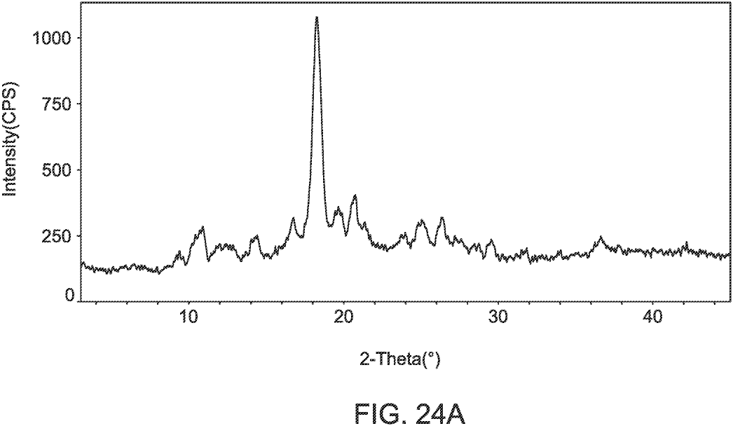

In some embodiments, the piperidine salt of Formula I (Form 1) exhibits an XRPD substantially as shown in FIG. 24. The XRPD of the piperidine salt of Formula I (Form 1) shown in FIG. 24 comprises reflection angles (degrees 2-theta ± 0.2 degrees 2-theta), line spacings (d values), and relative intensities as shown in Table 7:

TABLE 7

XRPD Data for crystalline form of the piperidine salt of Formula I (Formula IE-Form 1) shown in FIG. 24 .

| Angle (degrees 2-theta ± 0.2 degrees 2-theta) | d Value (Å) | Relative Intensity |
|---|---|---|
| 7.259 | 12.1682 | 100 |
| 9.319 | 9.4826 | 4.1 |
| 10.438 | 8.4684 | 3.8 |
| 12.24 | 7.225 | 21.4 |
| 14.279 | 6.1977 | 14.5 |
| 14.84 | 5.9647 | 11.8 |
| 16.1 | 5.5005 | 17.8 |
| 17.92 | 4.9457 | 45.1 |
| 19.779 | 4.485 | 23.4 |
| 20.6 | 4.308 | 21.7 |
| 21.32 | 4.164 | 6.9 |
| 22.14 | 4.0116 | 4.7 |
| 22.9 | 3.8803 | 27.1 |
| 24.32 | 3.6568 | 7.1 |
| 25.201 | 3.531 | 8.5 |
| 25.721 | 3.4608 | 8.2 |
| 26.92 | 3.3093 | 3.7 |
| 27.577 | 3.2318 | 4.8 |
| 28.82 | 3.0952 | 9.8 |
| 29.759 | 2.9996 | 6.3 |
| 30.7 | 2.9099 | 8.2 |
| 31.64 | 2.8255 | 5.1 |
| 32.541 | 2.7493 | 10 |
| 34.259 | 2.6153 | 4.6 |
| 35.398 | 2.5337 | 4.3 |
| 36.261 | 2.4754 | 5.4 |
| 36.88 | 2.4352 | 7 |
| 37.9 | 2.3719 | 3.8 |
| 39.917 | 2.2566 | 4.1 |
| 40.62 | 2.2192 | 3.8 |
| 43.279 | 2.0888 | 4.3 |

In some embodiments of the present disclosure, the piperidine salt of Formula I (Form 1) is characterized by an XRPD pattern comprising a peak at one of the angles listed in Table 7. In other aspects, the piperidine salt of Formula I (Form 1) is characterized by an XRPD pattern comprising more than one peak at one of the angles listed in Table 7 above. In other aspects, the piperidine salt of Formula I (Form 1) is characterized by an XRPD pattern comprising two peaks selected from the angles listed in Table 7 above. In other aspects, the piperidine salt of Formula I (Form 1) is characterized by an XRPD pattern comprising three peaks selected from the angles listed in Table 7 above. In other aspects, the piperidine salt of Formula I (Form 1) is characterized by an XRPD pattern comprising four peaks selected from the angles listed in Table 7 above. In other aspects, the piperidine salt of Formula I (Form 1) is characterized by an XRPD pattern comprising five peaks selected from the angles listed in Table 7 above. In other aspects, the piperidine salt of Formula I (Form 1) is characterized by an XRPD pattern comprising six peaks selected from the angles listed in Table 7 above. In other aspects, the piperidine salt of Formula I (Form 1) is characterized by an XRPD pattern comprising seven peaks selected from the angles listed in Table 7 above. In other aspects, the piperidine salt of Formula I (Form 1) is characterized by an XRPD pattern comprising eight peaks selected from the angles listed in Table 7 above. In other aspects, the piperidine salt of Formula I (Form 1) is characterized by an XRPD pattern comprising nine peaks selected from the angles listed in Table 7 above. In other aspects, the piperidine salt of Formula I (Form 1) is characterized by an XRPD pattern comprising ten peaks selected from the angles listed in Table 7 above. In other aspects, the piperidine salt of Formula I (Form 1) is characterized by an XRPD pattern comprising more than ten peaks selected from the angles listed in Table 7 above.

In some embodiments, the piperidine salt of Formula I (Form 1) is characterized by an XRPD pattern comprising peaks at 7.3, and 17.9 degrees ± 0.2 degrees 2-theta. In other embodiments, the piperidine salt of Formula I (Form 1) is characterized by an XRPD pattern comprising peaks at 7.3, 12.2, 16.1, and 17.9 degrees ± 0.2 degrees 2-theta. In other embodiments, the piperidine salt of Formula I (Form 1) is characterized by an XRPD pattern comprising peaks at 7.3, 12.2, 14.3, 14.8, 16.1, and 17.9 degrees ± 0.2 degree 2-theta. In other embodiments, the piperidine salt of Formula I (Form 1) is characterized by an XRPD pattern comprising peaks at 7.3, 12.2, 14.3, 14.8, 16.1, 17.9, and 19.8 degrees ± 0.2 degree 2-theta.. In other embodiments, the piperidine salt of Formula I (Form 1) is characterized by an XRPD pattern comprising peaks at 7.3, 12.2, 14.3, 14.8, 16.1, 17.9, 19.8, and 20.6 degrees ± 0.2 degree 2-theta.. In other embodiments, the piperidine salt of Formula I (Form 1) is characterized by an XRPD pattern comprising peaks at 7.3, 12.2, 14.3, 14.8, 16.1, 17.9, 19.8, 20.6, and 22.9 degrees ± 0.2 degree 2-theta.

In some embodiments of the present disclosure, the piperidine salt of Formula I (Form 1) is characterized by an XRPD pattern comprising peaks at two or more of 7.3, 12.2, 14.3, 14.8, 16.1, 17.9, 19.8, 20.6, and 22.9 degrees ± 0.2 degrees 2-theta.

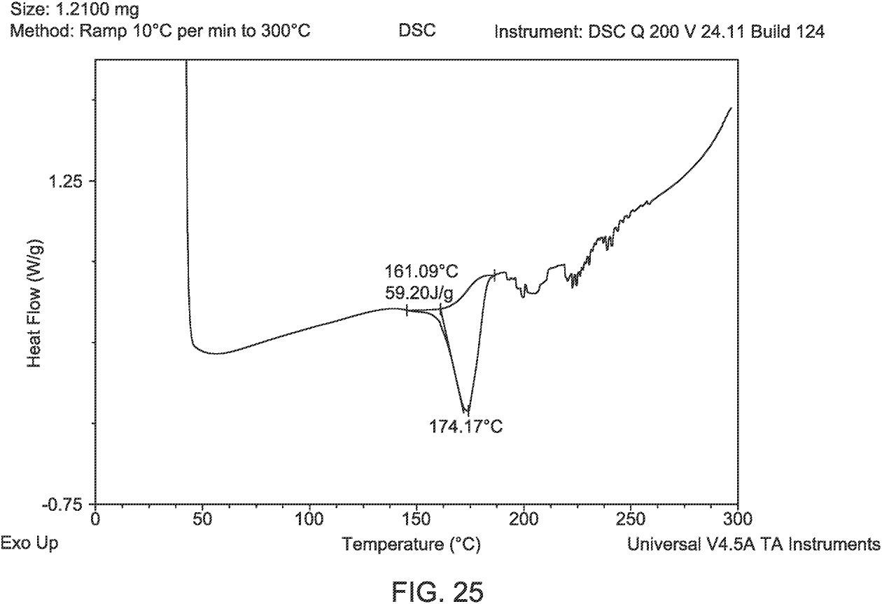

In some embodiments, the piperidine salt of Formula I (Form 1) can be characterized by a DSC thermogram substantially as shown in FIG. 25. As FIG. 25 shows, the piperidine salt of Formula I (Form 1) produced an endothermic peak at 174.17° C., with a peak onset temperature of 161.09° C., and an enthalpy of melting of 59.20 J/g, when heated at a rate of 10° C./min. In some embodiments of the present disclosure, the piperidine salt of Formula I (Form 1) is characterized by a DSC thermogram comprising an endothermic peak at about 174° C. In other embodiments of the present disclosure, the piperidine salt of Formula I (Form 1) is characterized by a DSC enthalpy of melting of about 59 J/g.

In some embodiments of the present disclosure, the piperidine salt of Formula I (Form 1) is characterized by an XRPD pattern comprising peaks at one or more of 7.3, 12.2, 14.3, 14.8, 16.1, 17.9, 19.8, 20.6, and 22.9 degrees ± 0.2 degrees 2-theta, and a DSC thermogram comprising an endothermic peak at about 174° C. when heated at a rate of 10° C./min.

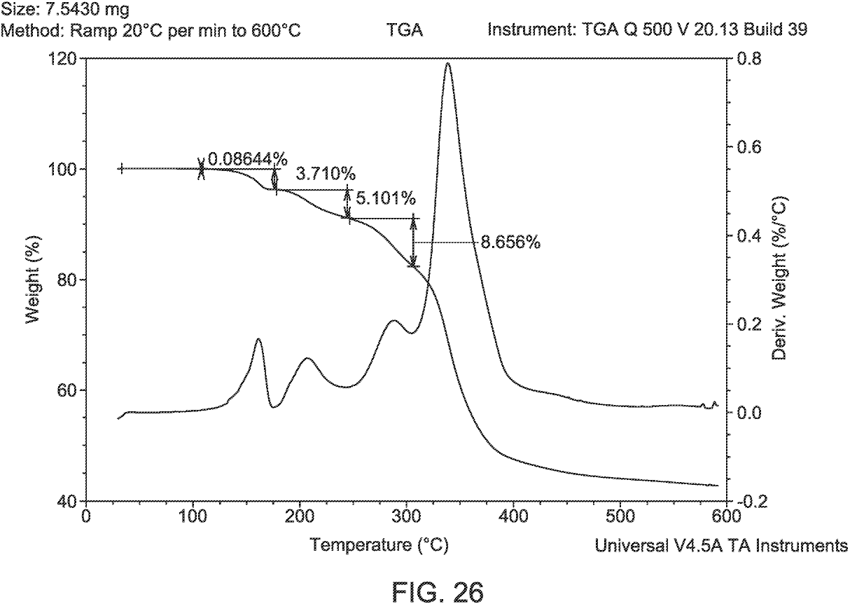

In some embodiments of the present disclosure, the piperidine salt of Formula I (Form 1) is characterized by an TGA profile substantially as shown in FIG. 26. As shown in FIG. 26, the piperidine salt of Formula I (Form 1) loses about 17.6% by weight upon heating to 300° C. at 20° C. per minute.

In some embodiments, the piperidine salt of Formula I (Form 2) exhibits an XRPD substantially as shown in FIG. 24A. The XRPD of the piperidine salt of Formula I (Form 2) shown in FIG. 24A comprises reflection angles (degrees 2-theta ± 0.2 degrees 2-theta), line spacings (d values), and relative intensities as shown in Table 7A:

TABLE 7A

XRPD Data for crystalline form of the piperidine salt of Formula I (Formula IE-Form 2) shown in FIG. 24A.

| Angle (degrees 2-theta ± 0.2 degrees 2-theta) | d Value (Å) | Relative Intensity |
|---|---|---|
| 9.362 | 9.4391 | 7.2 |
| 10.42 | 8.4828 | 13.1 |
| 10.861 | 8.139 | 16.4 |
| 11.98 | 7.3813 | 7.8 |
| 14.401 | 6.1454 | 10.8 |
| 16.759 | 5.2857 | 12 |
| 18.26 | 4.8544 | 100 |
| 19.718 | 4.4987 | 10.2 |
| 20.74 | 4.2791 | 20.3 |
| 21.396 | 4.1494 | 9.2 |
| 23.981 | 3.7078 | 7.3 |
| 25.041 | 3.5531 | 12.6 |
| 26.321 | 3.3832 | 13.3 |
| 28.759 | 3.1017 | 5.8 |
| 29.541 | 3.0213 | 9.1 |
| 31.838 | 2.8084 | 6.7 |
| 36.64 | 2.4506 | 9.2 |
| 42.182 | 2.1406 | 6.5 |

In some embodiments of the present disclosure, the piperidine salt of Formula I (Form 2) is characterized by an XRPD pattern comprising a peak at one of the angles listed in Table 7A. In other aspects, the piperidine salt of Formula I (Form 2) is characterized by an XRPD pattern comprising more than one peak at one of the angles listed in Table 7A above. In other aspects, the piperidine salt of Formula I (Form 2) is characterized by an XRPD pattern comprising two peaks selected from the angles listed in Table 7A above. In other aspects, the piperidine salt of Formula I (Form 2) is characterized by an XRPD pattern comprising three peaks selected from the angles listed in Table 7A above. In other aspects, the piperidine salt of Formula I (Form 2) is characterized by an XRPD pattern comprising four peaks selected from the angles listed in Table 7A above. In other aspects, the piperidine salt of Formula I (Form 2) is characterized by an XRPD pattern comprising five peaks selected from the angles listed in Table 7A above. In other aspects, the piperidine salt of Formula I (Form 2) is characterized by an XRPD pattern comprising six peaks selected from the angles listed in Table 7A above. In other aspects, the piperidine salt of Formula I (Form 2) is characterized by an XRPD pattern comprising seven peaks selected from the angles listed in Table 7A above. In other aspects, the piperidine salt of Formula I (Form 2) is characterized by an XRPD pattern comprising eight peaks selected from the angles listed in Table 7A above. In other aspects, the piperidine salt of Formula I (Form 2) is characterized by an XRPD pattern comprising nine peaks selected from the angles listed in Table 7A above. In other aspects, the piperidine salt of Formula I (Form 2) is characterized by an XRPD pattern comprising ten peaks selected from the angles listed in Table 7A above. In other aspects, the piperidine salt of Formula I (Form 2) is characterized by an XRPD pattern comprising more than ten peaks selected from the angles listed in Table 7A above.

In some embodiments, the piperidine salt of Formula I (Form 2) is characterized by an XRPD pattern comprising a peak at 18.3 degrees ± 0.2 degree 2-theta. In other embodiments, the piperidine salt of Formula I (Form 2) is characterized by an XRPD pattern comprising peaks at 16.8, and 18.3 degrees ± 0.2 degree 2-theta.. In other embodiments, the piperidine salt of Formula I (Form 2) is characterized by an XRPD pattern comprising peaks at 10.9, 16.8, and 18.3 degrees ± 0.2 degree 2-theta.. In other embodiments, the piperidine salt of Formula I (Form 2) is characterized by an XRPD pattern comprising peaks at 16.8, 18.3, and 20.7 degrees ± 0.2 degree 2-theta.

In some embodiments of the present disclosure, the piperidine salt of Formula I (Form 2) is characterized by an XRPD pattern comprising peaks at two or more of 10.9, 16.8, 18.3, and 20.7 degrees ± 0.2 degrees 2-theta.

In some embodiments, the disclosure is directed to a potassium salt of a compound of Formula I, having the formula IF:

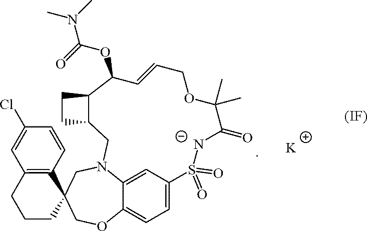

In some embodiments, the potassium salt of Formula I is substantially free of any other salt or solid form of Formula I.

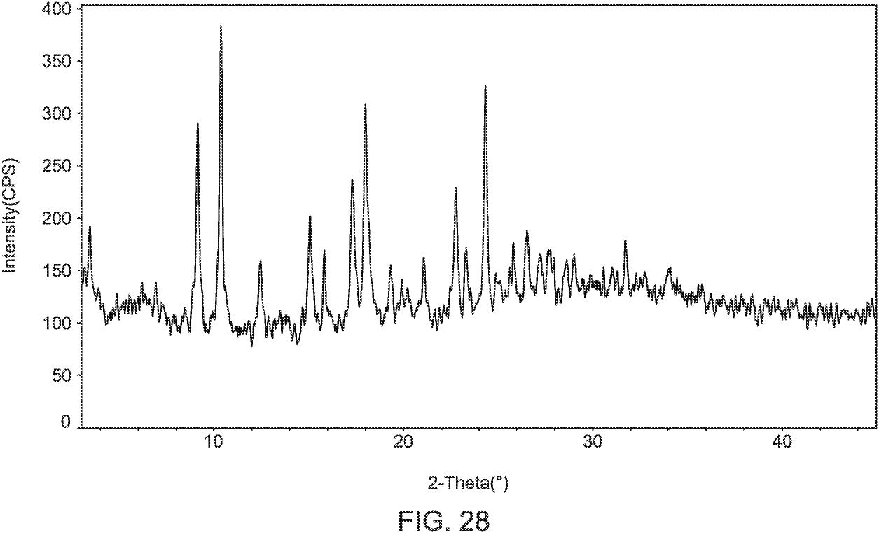

In some embodiments, the potassium salt of formula I exhibits an XRPD substantially as shown in FIG. 28. The XRPD of the potassium salt of formula I shown in FIG. 28 comprises reflection angles (degrees 2-theta ± 0.2 degrees 2-theta), line spacings (d values), and relative intensities as shown in Table 8:

TABLE 8

XRPD Data for crystalline form of the potassium salt of Formula I (Formula IF) shown in FIG. 28 .

| Angle (degrees 2-theta ± 0.2 degrees 2-theta) | d Value (Å) | Relative Intensity |
|---|---|---|
| 3.441 | 25.6562 | 20.4 |
| 6.215 | 14.2085 | 13.3 |
| 9.14 | 9.667 | 68.4 |
| 10.379 | 8.5159 | 100 |
| 12.46 | 7.0981 | 25.5 |
| 15.098 | 5.8634 | 39.8 |
| 17.32 | 5.1157 | 48.3 |
| 18.019 | 4.9189 | 70.7 |
| 19.339 | 4.586 | 18.4 |
| 20.221 | 4.3878 | 12.2 |
| 21.117 | 4.2037 | 19.7 |
| 22.78 | 3.9004 | 42.2 |
| 23.394 | 3.7994 | 13.9 |
| 24.359 | 3.6511 | 72.8 |
| 25.837 | 3.4455 | 19.4 |
| 26.541 | 3.3557 | 22.1 |
| 27.201 | 3.2757 | 15.3 |
| 27.778 | 3.209 | 17 |
| 31.741 | 2.8167 | 18.7 |

In some embodiments of the present disclosure, the potassium salt of Formula I is characterized by an XRPD pattern comprising a peak at one of the angles listed in Table 8. In other aspects, the potassium salt of Formula I is characterized by an XRPD pattern comprising more than one peak at one of the angles listed in Table 8 above. In other aspects, the potassium salt of Formula I is characterized by an XRPD pattern comprising two peaks selected from the angles listed in Table 8 above. In other aspects, the potassium salt of Formula I is characterized by an XRPD pattern comprising three peaks selected from the angles listed in Table 8 above. In other aspects, the potassium salt of Formula I is characterized by an XRPD pattern comprising four peaks selected from the angles listed in Table 8 above. In other aspects, the potassium salt of Formula I is characterized by an XRPD pattern comprising five peaks selected from the angles listed in Table 8 above. In other aspects, the potassium salt of Formula I is characterized by an XRPD pattern comprising six peaks selected from the angles listed in Table 8 above. In other aspects, the potassium salt of Formula I is characterized by an XRPD pattern comprising seven peaks selected from the angles listed in Table 8 above. In other aspects, the potassium salt of Formula I is characterized by an XRPD pattern comprising eight peaks selected from the angles listed in Table 8 above. In other aspects, the potassium salt of Formula I is characterized by an XRPD pattern comprising nine peaks selected from the angles listed in Table 8 above. In other aspects, the potassium salt of Formula I is characterized by an XRPD pattern comprising ten peaks selected from the angles listed in Table 8 above. In other aspects, the potassium salt of Formula I is characterized by an XRPD pattern comprising more than ten peaks selected from the angles listed in Table 8 above.

In some embodiments, the potassium salt of Formula I is characterized by an XRPD pattern comprising peaks at 9.1, 10.4, 18.0, and 19.3 degrees ± 0.2 degrees 2-theta. In other embodiments, the potassium salt of Formula I is characterized by an XRPD pattern comprising peaks at 10.4, 18.0, 19.3, 22.8, and 24.4 degrees ± 0.2 degrees 2-theta. In other embodiments, the potassium salt of Formula I is characterized by an XRPD pattern comprising peaks at 9.1, 10.4, 19.3, and 22.8 degrees ± 0.2 degree 2-theta. In other embodiments, the potassium salt of Formula I is characterized by an XRPD pattern comprising peaks at 9.1, 10.4, 18.0, 19.3, and 24.4 degrees ± 0.2 degree 2-theta.. In other embodiments, the potassium salt of Formula I is characterized by an XRPD pattern comprising peaks at 9.1, 10.4, 18.0, 19.3, 22.8, and 24.4 degrees ± 0.2 degree 2-theta.. In other embodiments, the potassium salt of Formula I is characterized by an XRPD pattern comprising peaks at 9.1, 10.4, 15.1, 18.0, 19.3, 22.8, and 24.4 degrees ± 0.2 degree 2-theta.

In some embodiments of the present disclosure, the potassium salt of Formula I is characterized by an XRPD pattern comprising peaks at two or more of 9.1, 10.4, 12.5, 15.1, 18.0, 19.3, 22.8, and 24.4 degrees ± 0.2 degrees 2-theta.

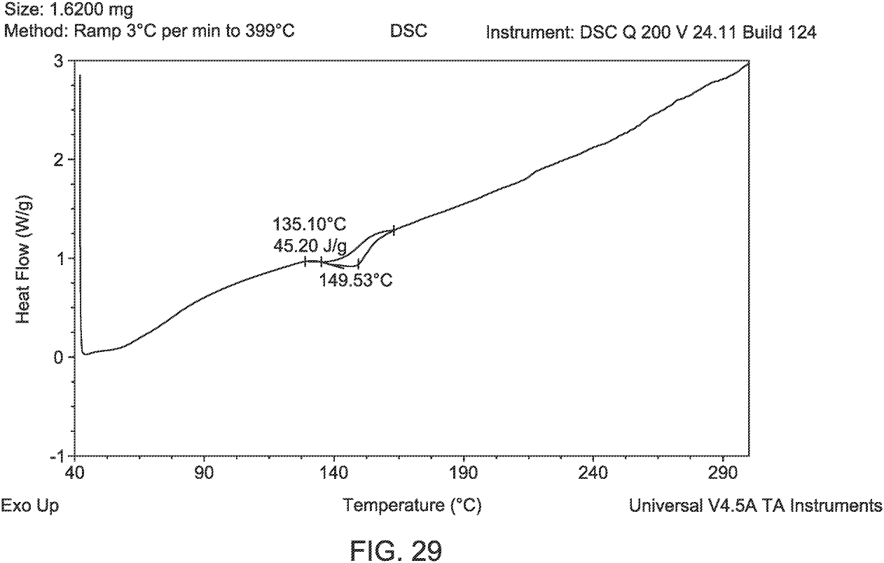

In some embodiments, the potassium salt of Formula I can be characterized by a DSC thermogram substantially as shown in FIG. 29. As FIG. 29 shows, the potassium salt of Formula I produced an endothermic peak at 149.53° C., with a peak onset temperature of 135.10° C., and an enthalpy of melting of 45.20 J/g, when heated at a rate of 10° C./min. In some embodiments of the present disclosure, the potassium salt of Formula I is characterized by a DSC thermogram comprising an endothermic peak at about 150° C. In other embodiments of the present disclosure, the potassium salt of Formula I is characterized by a DSC enthalpy of melting of about 45 J/g.

In some embodiments of the present disclosure, the potassium salt of Formula I is characterized by an XRPD pattern comprising peaks at one or more of 9.1, 10.4, 12.5, 15.1, 18.0, 19.3, 22.8, and 24.4 degrees ± 0.2 degrees 2-theta, and a DSC thermogram comprising an endothermic peak at about 150° C. when heated at a rate of 10° C./min.

In some embodiments, the disclosure is directed to a (S)-(-)-α-Methylbenzylamine salt of a compound of Formula I, having the formula IG:

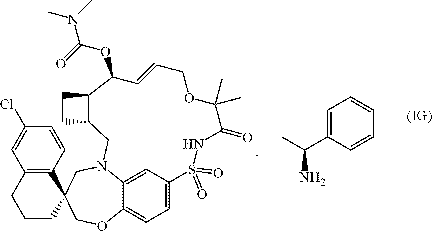

In some embodiments, the disclosure is directed to a crystalline form of the (S)-(-)-α-methylbenzylamine salt of Formula I.

In some embodiments, the (S)-(-)-α-methylbenzylamine salt of Formula I is substantially free of any other salt or solid form of Formula I.

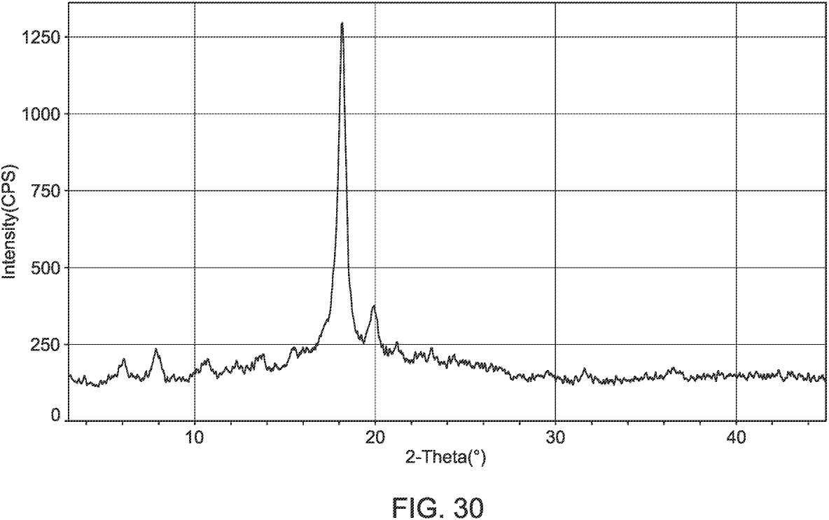

In some embodiments, the (S)-(-)-α-Methylbenzylamine salt of formula I exhibits an XRPD substantially as shown in FIG. 30. The XRPD of the (S)-(-)-α-Methylbenzylamine salt of formula I shown in FIG. 30 comprises reflection angles (degrees 2-theta ± 0.2 degrees 2-theta), line spacings (d values), and relative intensities as shown in Table 9:

TABLE 9

XRPD Data for crystalline form of the (S)-(-)-α-Methylbenzylamine salt of Formula I (Formula IG) shown in FIG. 30 .

| Angle (degrees 2-theta ± 0.2 degrees 2-theta) | d Value (Å) | Relative Intensity |
|---|---|---|
| 6.08 | 14.5254 | 7.4 |
| 7.841 | 11.2663 | 10.1 |
| 10.72 | 8.2458 | 6 |
| 13.781 | 6.4206 | 5.3 |
| 18.18 | 4.8757 | 100 |
| 19.94 | 4.4492 | 12.9 |
| 23.175 | 3.8349 | 4.8 |
| 31.619 | 2.8273 | 4.7 |

In some embodiments of the present disclosure, the (S)-(-)-α-Methylbenzylamine salt of Formula I is characterized by an XRPD pattern comprising a peak at one of the angles listed in Table 9. In other aspects, the (S)-(-)-α-Methylbenzylamine salt of Formula I is characterized by an XRPD pattern comprising more than one peak at one of the angles listed in Table 9 above. In other aspects, the (S)-(-)-α-Methylbenzylamine salt of Formula I is characterized by an XRPD pattern comprising two peaks selected from the angles listed in Table 9 above. In other aspects, the (S)-(-)-α-Methylbenzylamine salt of Formula I is characterized by an XRPD pattern comprising three peaks selected from the angles listed in Table 9 above. In other aspects, the (S)-(-)-α-Methylbenzylamine salt of Formula I is characterized by an XRPD pattern comprising four peaks selected from the angles listed in Table 9 above. In other aspects, the (S)-(-)-α-Methylbenzylamine salt of Formula I is characterized by an XRPD pattern comprising five peaks selected from the angles listed in Table 9 above. In other aspects, the (S)-(-)-α-Methylbenzylamine salt of Formula I is characterized by an XRPD pattern comprising six peaks selected from the angles listed in Table 9 above. In other aspects, the (S)-(-)-α-Methylbenzylamine salt of Formula I is characterized by an XRPD pattern comprising seven peaks selected from the angles listed in Table 9 above. In other aspects, the (S)-(-)-α-Methylbenzylamine salt of Formula I is characterized by an XRPD pattern comprising eight peaks selected from the angles listed in Table 9 above.

In some embodiments, the (S)-(-)-α-Methylbenzylamine salt of Formula I is characterized by an XRPD pattern comprising a peaks at 18.2 degrees ± 0.2 degrees 2-theta. In other embodiments, the (S)-(-)-α-Methylbenzylamine salt of Formula I is characterized by an XRPD pattern comprising a peak at 19.9 degrees ± 0.2 degrees 2-theta. In other embodiments, the (S)-(-)-α-Methylbenzylamine salt of Formula I is characterized by an XRPD pattern comprising peaks at 18.2 and 19.9 degrees ± 0.2 degree 2-theta.

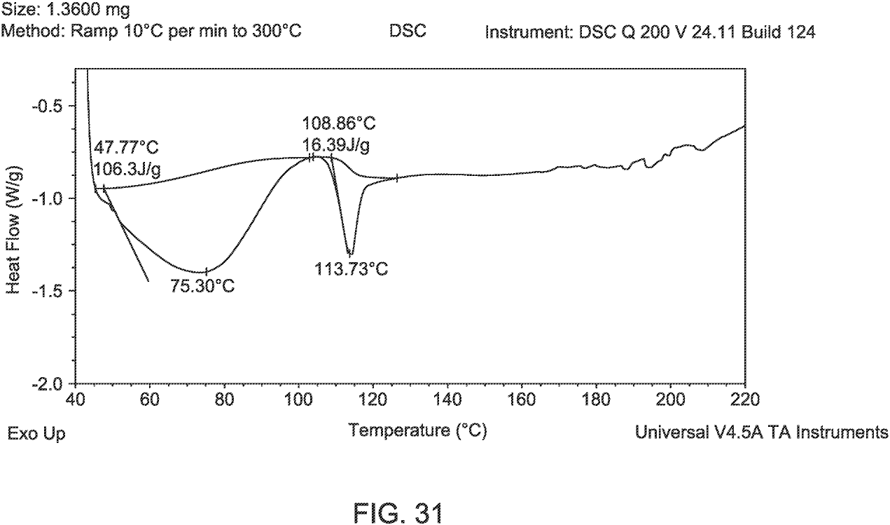

In some embodiments, the (S)-(-)-α-Methylbenzylamine salt of Formula I can be characterized by a DSC thermogram substantially as shown in FIG. 31. As FIG. 31 shows, the (S)-(-)-α-Methylbenzylamine salt of Formula I produced an endothermic peak at 75.30° C., with a peak onset temperature of 47.77° C., and an enthalpy of melting of 106.3 J/g, followed by an endothermic peak at 113.73° C., with a peak onset temperature of 108.86° C., and an enthalpy of melting of 16.39 J/g when heated at a rate of 10° C./min. In some embodiments of the present disclosure, the (S)-(-)-α-Methylbenzylamine salt of Formula I is characterized by a DSC thermogram comprising an endothermic peak at about 75° C. In other embodiments of the present disclosure, the (S)-(-)-α-Methylbenzylamine salt of Formula I is characterized by a DSC enthalpy of melting of about 106.3 J/g. In some embodiments of the present disclosure, the (S)-(-)-α-Methylbenzylamine salt of Formula I is characterized by a DSC thermogram comprising an endothermic peak at about 114° C. In other embodiments of the present disclosure, the (S)-(-)-α-Methylbenzylamine salt of Formula I is characterized by a DSC enthalpy of melting of about 16.4 J/g.

In some embodiments of the present disclosure, the (S)-(-)-α-Methylbenzylamine salt of Formula I is characterized by an XRPD pattern comprising peaks at one or more of 18.2 and 19.9 degrees ± 0.2 degrees 2-theta, and a DSC thermogram comprising an endothermic peak at about 75° C. or at about 114° C. when heated at a rate of 10° C./min.

In some embodiments, the disclosure is directed to an ethylenediamine salt of the compound of Formula I, having formula IH:

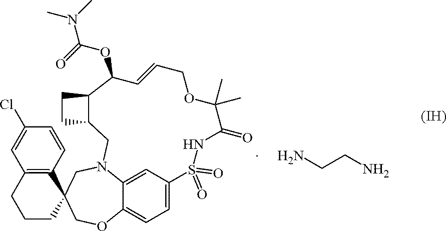

In some embodiments, the disclosure is directed to a crystalline form of an ethylenediamine salt of the compound of Formula I.

In some embodiments, the ethylenediamine salt of Formula I is substantially free of any other salt or solid form of Formula I.

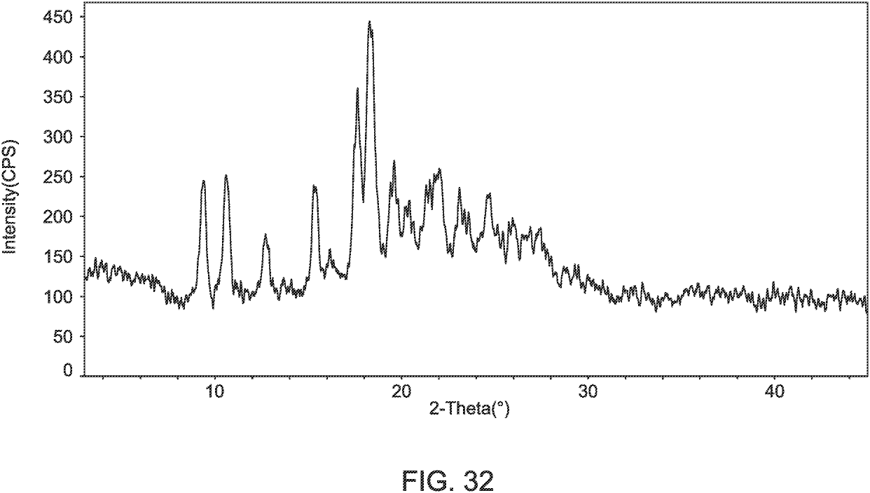

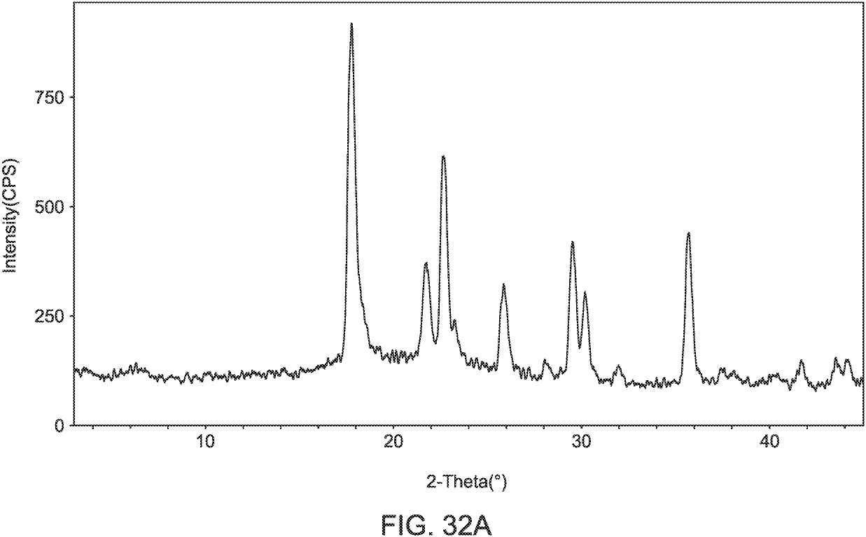

In some embodiments, the ethylenediamine salt of formula I (Form 1) exhibits an XRPD substantially as shown in FIG. 32. The XRPD of the ethylenediamine salt of formula I (Form 1) shown in FIG. 32 comprises reflection angles (degrees 2-theta ± 0.2 degrees 2-theta), line spacings (d values), and relative intensities as shown in Table 10:

TABLE 10

XRPD Data for crystalline form of the ethylenediamine salt of Formula I (Formula IH-Form 1) shown in FIG. 32 .

| Angle (degrees 2-theta ± 0.2 degrees 2-theta) | d Value (Å) | Relative Intensity |
|---|---|---|
| 9.419 | 9.3814 | 1.5 |
| 10.6 | 8.3391 | 52.8 |
| 12.758 | 6.9329 | 24.9 |
| 15.38 | 5.7564 | 39.3 |
| 17.659 | 5.0183 | 75.1 |
| 18.3 | 4.844 | 100 |
| 19.619 | 4.521 | 38.4 |
| 20.421 | 4.3455 | 20.3 |
| 21.521 | 4.1256 | 29.2 |
| 22.04 | 4.0297 | 34.4 |
| 23.12 | 3.8439 | 26.9 |
| 23.602 | 3.7664 | 16.1 |
| 24.759 | 3.593 | 25.6 |
| 25.961 | 3.4292 | 17.4 |
| 26.862 | 3.3162 | 15.7 |
| 27.495 | 3.2414 | 16.4 |
| 39.96 | 2.2543 | 11.8 |

In some embodiments of the present disclosure, the ethylenediamine salt of Formula I (Form 1) is characterized by an XRPD pattern comprising a peak at one of the angles listed in Table 10. In other aspects, the ethylenediamine salt of Formula I (Form 1) is characterized by an XRPD pattern comprising more than one peak at one of the angles listed in Table 10 above. In other aspects, the ethylenediamine salt of Formula I (Form 1) is characterized by an XRPD pattern comprising two peaks selected from the angles listed in Table 10 above. In other aspects, the ethylenediamine salt of Formula I (Form 1) is characterized by an XRPD pattern comprising three peaks selected from the angles listed in Table 10 above. In other aspects, the ethylenediamine salt of Formula I (Form 1) is characterized by an XRPD pattern comprising four peaks selected from the angles listed in Table 10 above. In other aspects, the ethylenediamine salt of Formula I (Form 1) is characterized by an XRPD pattern comprising five peaks selected from the angles listed in Table 10 above. In other aspects, the ethylenediamine salt of Formula I (Form 1) is characterized by an XRPD pattern comprising six peaks selected from the angles listed in Table 10 above. In other aspects, the ethylenediamine salt of Formula I (Form 1) is characterized by an XRPD pattern comprising seven peaks selected from the angles listed in Table 10 above. In other aspects, the ethylenediamine salt of Formula I (Form 1) is characterized by an XRPD pattern comprising eight peaks selected from the angles listed in Table 10 above. In other aspects, the ethylenediamine salt of Formula I (Form 1) is characterized by an XRPD pattern comprising nine peaks selected from the angles listed in Table 10 above. In other aspects, the ethylenediamine salt of Formula I (Form 1) is characterized by an XRPD pattern comprising ten peaks selected from the angles listed in Table 10 above. In other aspects, the ethylenediamine salt of Formula I (Form 1) is characterized by an XRPD pattern comprising more than ten peaks selected from the angles listed in Table 10 above.

In some embodiments, the ethylenediamine salt of Formula I (Form 1) is characterized by an XRPD pattern comprising a peak at 9.4, 10.6, 17.7, and 18.3 degrees ± 0.2 degrees 2-theta. In other embodiments, the ethylenediamine salt of Formula I (Form 1) is characterized by an XRPD pattern comprising peaks at 9.4, 10.6, 15.4, 17.7, and 18.3 degrees ± 0.2 degrees 2-theta. In other embodiments, the ethylenediamine salt of Formula I (Form 1) is characterized by an XRPD pattern comprising peaks at 9.4, 10.6, 15.4, 17.7, 18.3, and 19.6 degrees ± 0.2 degree 2-theta. In other embodiments, the ethylenediamine salt of Formula I (Form 1) is characterized by an XRPD pattern comprising peaks at 9.4, 10.6, 15.4, 17.7, 18.3, 19.6, and 22.0 degrees ± 0.2 degree 2-theta.. In other embodiments, the ethylenediamine salt of Formula I (Form 1) is characterized by an XRPD pattern comprising peaks at 9.4, 10.6, 15.4, 17.7, 18.3, 19.6, 22.0, and 23.1 degrees ± 0.2 degree 2-theta.. In other embodiments, the ethylenediamine salt of Formula I (Form 1) is characterized by an XRPD pattern comprising peaks at 9.4, 10.6, 15.4, 17.7, 18.3, 19.6, 22.0, 23.1, and 24.8 degrees ± 0.2 degree 2-theta.

In some embodiments of the present disclosure, the ethylenediamine salt of Formula I (Form 1) is characterized by an XRPD pattern comprising peaks at two or more of 9.4, 10.6, 15.4, 17.7, 18.3, 19.6, 22.0, 23.1, and 24.8 degrees ± 0.2 degrees 2-theta.

In other embodiments, the ethylenediamine salt of formula I (Form 2) exhibits an XRPD substantially as shown in FIG. 32A. The XRPD of the ethylenediamine salt of formula I (Form 2) shown in FIG. 32A comprises reflection angles (degrees 2-theta ± 0.2 degrees 2-theta), line spacings (d values), and relative intensities as shown in Table 10:

TABLE 11

XRPD Data for crystalline form of the ethylenediamine salt of Formula I (Formula IH-Form 2) shown in FIG. 32A.

| Angle (degrees 2-theta ± 0.2 degrees 2-theta) | d Value (Å) | Relative Intensity |
|---|---|---|
| 17.76 | 4.9899 | 100 |
| 21.759 | 4.0811 | 25.5 |
| 22.66 | 3.9208 | 60.5 |
| 25.86 | 3.4424 | 26.7 |
| 29.52 | 3.0234 | 41.2 |
| 30.2 | 2.9569 | 26 |
| 31.98 | 2.7962 | 5.9 |
| 35.719 | 2.5116 | 45.4 |
| 41.718 | 2.1633 | 8.5 |
| 43.539 | 2.0769 | 9 |
| 44.199 | 2.0474 | 7.4 |

In some embodiments of the present disclosure, the ethylenediamine salt of Formula I (Form 2) is characterized by an XRPD pattern comprising a peak at one of the angles listed in Table 11. In other aspects, the ethylenediamine salt of Formula I (Form 2) is characterized by an XRPD pattern comprising more than one peak at one of the angles listed in Table 11 above. In other aspects, the ethylenediamine salt of Formula I (Form 2) is characterized by an XRPD pattern comprising two peaks selected from the angles listed in Table 11 above. In other aspects, the ethylenediamine salt of Formula I (Form 2) is characterized by an XRPD pattern comprising three peaks selected from the angles listed in Table 11 above. In other aspects, the ethylenediamine salt of Formula I (Form 2) is characterized by an XRPD pattern comprising four peaks selected from the angles listed in Table 11 above. In other aspects, the ethylenediamine salt of Formula I (Form 2) is characterized by an XRPD pattern comprising five peaks selected from the angles listed in Table 11 above. In other aspects, the ethylenediamine salt of Formula I (Form 2) is characterized by an XRPD pattern comprising six peaks selected from the angles listed in Table 11 above. In other aspects, the ethylenediamine salt of Formula I (Form 2) is characterized by an XRPD pattern comprising seven peaks selected from the angles listed in Table 11 above. In other aspects, the ethylenediamine salt of Formula I (Form 2) is characterized by an XRPD pattern comprising eight peaks selected from the angles listed in Table 11 above. In other aspects, the ethylenediamine salt of Formula I (Form 2) is characterized by an XRPD pattern comprising nine peaks selected from the angles listed in Table 11 above. In other aspects, the ethylenediamine salt of Formula I (Form 2) is characterized by an XRPD pattern comprising ten peaks selected from the angles listed in Table 11 above. In other aspects, the ethylenediamine salt of Formula I (Form 2) is characterized by an XRPD pattern comprising more than ten peaks selected from the angles listed in Table 11 above.

In some embodiments, the ethylenediamine salt of Formula I (Form 2) is characterized by an XRPD pattern comprising a peak at 17.8 degrees ± 0.2 degrees 2-theta. In other embodiments, the ethylenediamine salt of Formula I (Form 2) is characterized by an XRPD pattern comprising peaks at 17.8, and 21.8 degrees ± 0.2 degrees 2-theta. In other embodiments, the ethylenediamine salt of Formula I (Form 2) is characterized by an XRPD pattern comprising peaks at 17.8, 21.8, and 22.7 degrees ± 0.2 degree 2-theta. In other embodiments, the ethylenediamine salt of Formula I (Form 2) is characterized by an XRPD pattern comprising peaks at 17.8, 21.8, 22.7, and 25.9 degrees ± 0.2 degree 2-theta.. In other embodiments, the ethylenediamine salt of Formula I (Form 2) is characterized by an XRPD pattern comprising peaks at 17.8, 21.8, 22.7, 25.9, and 29.5 degrees ± 0.2 degree 2-theta.. In other embodiments, the ethylenediamine salt of Formula I (Form 2) is characterized by an XRPD pattern comprising peaks at 17.8, 21.8, 22.7, 25.9, 29.5, and 35.7 degrees ± 0.2 degree 2-theta.

In some embodiments of the present disclosure, the ethylenediamine salt of Formula I (Form 2) is characterized by an XRPD pattern comprising peaks at two or more of 17.8, 21.8, 22.7, 25.9, 29.5, and 35.7 degrees ± 0.2 degrees 2-theta.

In some embodiments, the disclosure is directed to a 4-((2-aminoethyl)amino)-4-methylpentan-2-one salt of a compound of Formula I, having formula IK:

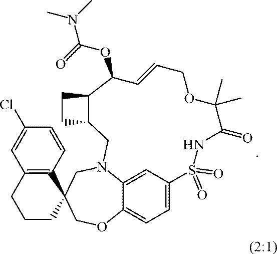

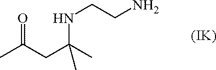

In some embodiments, the disclosure is directed to a crystalline form of the 4-((2-aminoethyl)amino)-4-methylpentan-2-one salt of Formula I.

In some embodiments, the 4-((2-aminoethyl)amino)-4-methylpentan-2-one salt of Formula I is substantially free of any other salt or solid form of Formula I.

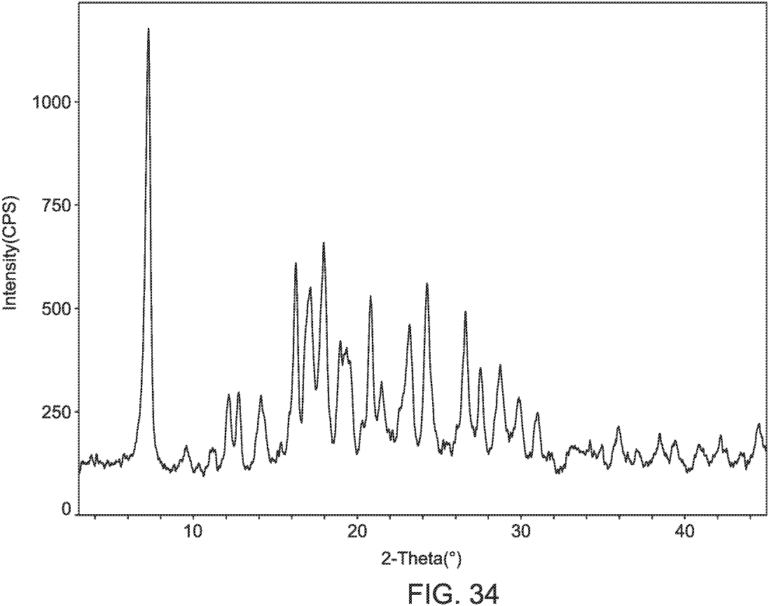

In some embodiments, the 4-((2-aminoethyl)amino)-4-methylpentan-2-one salt of formula I exhibits an XRPD substantially as shown in FIG. 34. The XRPD of the 4-((2-aminoethyl)amino)-4-methylpentan-2-one salt of formula I shown in FIG. 34 comprises reflection angles (degrees 2-theta ± 0.2 degrees 2-theta), line spacings (d values), and relative intensities as shown in Table 12:

TABLE 12

XRPD Data for crystalline form of the 4-((2-aminoethyl)amino)-4-methylpentan-2-one salt of Formula I (Formula IK) shown in FIG. 34 .

| Angle (degrees 2-theta ± 0.2 degrees 2-theta) | d Value (Å) | Relative Intensity |
|---|---|---|
| 7.26 | 12.1665 | 100 |
| 9.638 | 9.169 | 5.2 |
| 11.18 | 7.9079 | 5.6 |
| 12.16 | 7.2722 | 17.8 |
| 12.76 | 6.9317 | 17.7 |
| 14.14 | 6.2585 | 16.2 |
| 16.26 | 5.4469 | 37.8 |
| 17.159 | 5.1633 | 36.5 |
| 17.96 | 4.9348 | 44.2 |
| 19.52 | 4.5438 | 20.5 |
| 20.82 | 4.2629 | 35.1 |
| 21.499 | 4.1298 | 14.6 |
| 23.2 | 3.8307 | 27.3 |
| 24.28 | 3.6628 | 37.7 |
| 26.62 | 3.3458 | 32.8 |
| 27.54 | 3.2361 | 17.2 |
| 28.74 | 3.1037 | 20 |
| 29.88 | 2.9878 | 11.9 |
| 31.02 | 2.8805 | 11.3 |
| 34.961 | 2.5644 | 4.2 |
| 35.979 | 2.4941 | 9.4 |
| 38.556 | 2.3331 | 6 |
| 39.42 | 2.2839 | 6.5 |
| 40.86 | 2.2067 | 5.4 |
| 42.274 | 2.1361 | 5.5 |

In some embodiments of the present disclosure, the 4-((2-aminoethyl)amino)-4-methylpentan-2-one salt of Formula I is characterized by an XRPD pattern comprising a peak at one of the angles listed in Table 12. In other aspects, the 4-((2-aminoethyl)amino)-4-methylpentan-2-one salt of Formula I is characterized by an XRPD pattern comprising more than one peak at one of the angles listed in Table 12 above. In other aspects, the 4-((2-aminoethyl)amino)-4-methylpentan-2-one salt of Formula I is characterized by an XRPD pattern comprising two peaks selected from the angles listed in Table 12 above. In other aspects, the 4-((2-aminoethyl)amino)-4-methylpentan-2-one salt of Formula I is characterized by an XRPD pattern comprising three peaks selected from the angles listed in Table 12 above. In other aspects, the 4-((2-aminoethyl)amino)-4-methylpentan-2-one salt of Formula I is characterized by an XRPD pattern comprising four peaks selected from the angles listed in Table 12 above. In other aspects, the 4-((2-aminoethyl)amino)-4-methylpentan-2-one salt of Formula I is characterized by an XRPD pattern comprising five peaks selected from the angles listed in Table 12 above. In other aspects, the 4-((2-aminoethyl)amino)-4-methylpentan-2-one salt of Formula I is characterized by an XRPD pattern comprising six peaks selected from the angles listed in Table 12 above. In other aspects, the 4-((2-aminoethyl)amino)-4-methylpentan-2-one salt of Formula I is characterized by an XRPD pattern comprising seven peaks selected from the angles listed in Table 12 above. In other aspects, the 4-((2-aminoethyl)amino)-4-methylpentan-2-one salt of Formula I is characterized by an XRPD pattern comprising eight peaks selected from the angles listed in Table 12 above. In other aspects, the 4-((2-aminoethyl)amino)-4-methylpentan-2-one salt of Formula I is characterized by an XRPD pattern comprising nine peaks selected from the angles listed in Table 12 above. In other aspects, the 4-((2-aminoethyl)amino)-4-methylpentan-2-one salt of Formula I is characterized by an XRPD pattern comprising ten peaks selected from the angles listed in Table 12 above. In other aspects, the 4-((2-aminoethyl)amino)-4-methylpentan-2-one salt of Formula I is characterized by an XRPD pattern comprising more than ten peaks selected from the angles listed in Table 12 above.

In some embodiments, the 4-((2-aminoethyl)amino)-4-methylpentan-2-one salt of Formula I is characterized by an XRPD pattern comprising peaks at 16.3, 17.2, and 18.0 degrees ± 0.2 degrees 2-theta. In other embodiments, the 4-((2-aminoethyl)amino)-4-methylpentan-2-one salt of Formula I is characterized by an XRPD pattern comprising peaks at 12.2, 12.8, 16.3, 17.2, 18.0, and 20.8 degrees ± 0.2 degrees 2-theta. In other embodiments, the 4-((2-aminoethyl)amino)-4-methylpentan-2-one salt of Formula I is characterized by an XRPD pattern comprising peaks at 16.3, 17.2, 18.0, 20.8, 23.2, 24.3, and 26.6 degrees ± 0.2 degree 2-theta. In other embodiments, the 4-((2-aminoethyl)amino)-4-methylpentan-2-one salt of Formula I is characterized by an XRPD pattern comprising peaks at 7.3, 12.2, 12.8, 16.3, and 17.2 degrees ± 0.2 degree 2-theta.. In other embodiments, the 4-((2-aminoethyl)amino)-4-methylpentan-2-one salt of Formula I is characterized by an XRPD pattern comprising peaks at 7.3, 12.2, 12.8, 16.3, 17.2, 18.0, 20.8, and 23.2 degrees ± 0.2 degree 2-theta. In other embodiments, the 4-((2-aminoethyl)amino)-4-methylpentan-2-one salt f Formula I is characterized by an XRPD pattern comprising peaks at 7.3, 12.2, 12.8, 16.3, 17.2, 18.0, 20.8, 23.2, 24.3, and 26.6 degrees ± 0.2 degree 2-theta.

In some embodiments of the present disclosure, the 4-((2-aminoethyl)amino)-4-methylpentan-2-one salt of Formula I is characterized by an XRPD pattern comprising peaks at two or more of 7.3, 12.2, 12.8, 16.3, 17.2, 18.0, 20.8, 23.2, 24.3, and 26.6 degrees ± 0.2 degrees 2-theta.

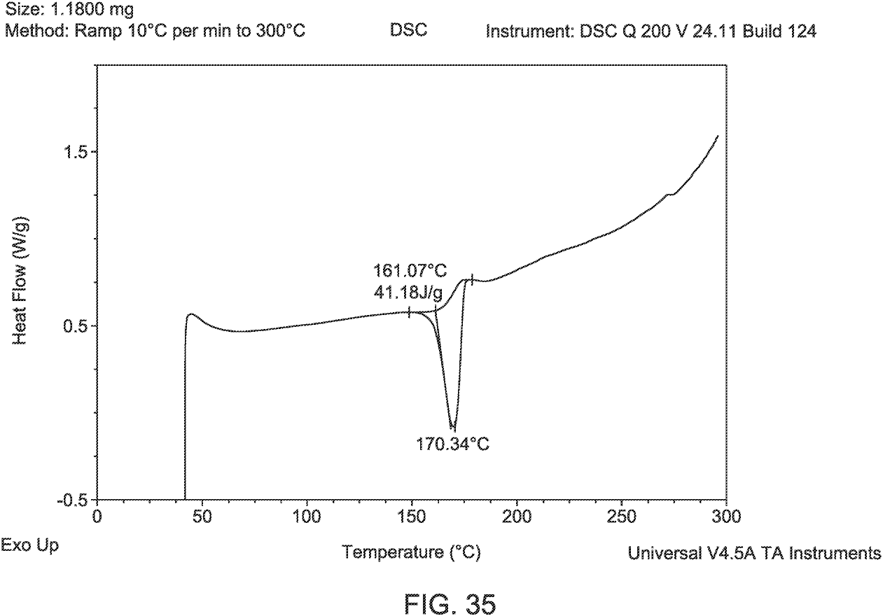

In some embodiments, the 4-((2-aminoethyl)amino)-4-methylpentan-2-one salt of Formula I can be characterized by a DSC thermogram substantially as shown in FIG. 35. As FIG. 35 shows, the 4-((2-aminoethyl)amino)-4-methylpentan-2-one salt of Formula I produced an endothermic peak at 170.34° C., with a peak onset temperature of 161.07° C., and an enthalpy of melting of 41.18 J/g, when heated at a rate of 10° C./min. In some embodiments of the present disclosure, the 4-((2-aminoethyl)amino)-4-methylpentan-2-one salt of Formula I is characterized by a DSC thermogram comprising an endothermic peak at about 170° C. In other embodiments of the present disclosure, the 4-((2-aminoethyl)amino)-4-methylpentan-2-one salt of Formula I is characterized by a DSC enthalpy of melting of about 41 J/g.

In some embodiments of the present disclosure, the 4-((2-aminoethyl)amino)-4-methylpentan-2-one salt of Formula I is characterized by an XRPD pattern comprising peaks at one or more of 7.3, 12.2, 12.8, 16.3, 17.2, 18.0, 20.8, 23.2, 24.3, and 26.6 degrees ± 0.2 degrees 2-theta, and a DSC thermogram comprising an endothermic peak at about 170° C. when heated at a rate of 10° C./min.

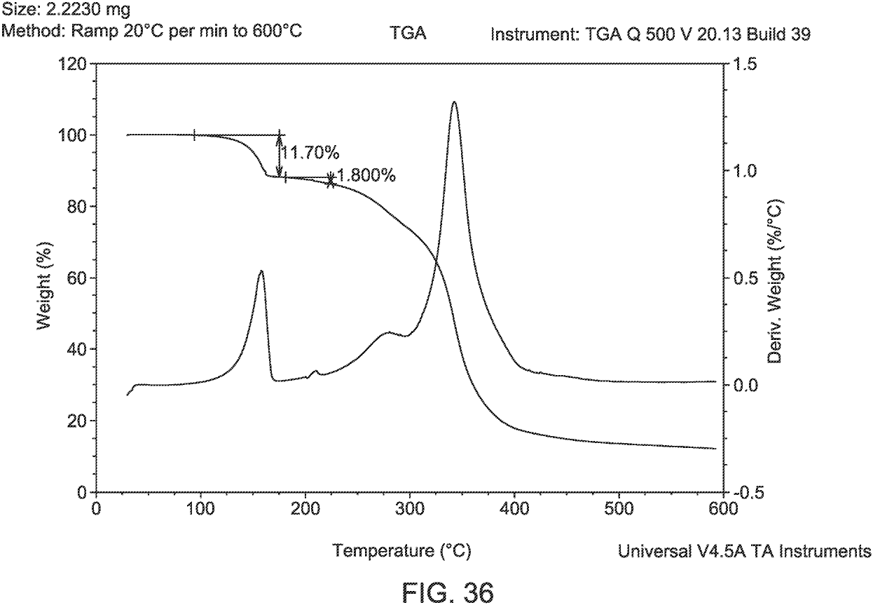

In some embodiments of the present disclosure, the 4-((2-aminoethyl)amino)-4-methylpentan-2-one salt of Formula I is characterized by an TGA profile substantially as shown in FIG. 36. As shown in FIG. 36, the 4-((2-aminoethyl)amino)-4-methylpentan-2-one salt of Formula I loses about 13.5% by weight upon heating to 250° C. at 20° C. per minute.

### Pharmaceutical Compositions and Methods of Administration

The subject pharmaceutical compositions are typically formulated to provide a therapeutically effective amount of a compound of the present disclosure as the active ingredient, or a pharmaceutically acceptable salt, ester, prodrug, solvate, hydrate or derivative thereof. Where desired, the pharmaceutical compositions contain pharmaceutically acceptable salt and/or coordination complex thereof, and one or more pharmaceutically acceptable excipients, carriers, including inert solid diluents and fillers, diluents, including sterile aqueous solution and various organic solvents, permeation enhancers, solubilizers and adjuvants.

The subject pharmaceutical compositions can be administered alone or in combination with one or more other agents, which are also typically administered in the form of pharmaceutical compositions. Where desired, the one or more compounds of the invention and other agent(s) may be mixed into a preparation or both components may be formulated into separate preparations to use them in combination separately or at the same time.

In some embodiments, the concentration of one or more compounds provided in the pharmaceutical compositions of the present invention is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% (or a number in the range defined by and including any two numbers above) w/w, w/v or v/v.

In some embodiments, the concentration of one or more compounds of the invention is greater than 90%, 80%, 70%, 60%, 50%, 40%, 30%, 20%, 19.75%, 19.50%, 19.25%, 19%, 18.75%, 18.50%, 18.25% 18%, 17.75%, 17.50%, 17.25% 17%, 16.75%, 16.50%, 16.25%, 16%, 15.75%, 15.50%, 15.25% 15%, 14.75%, 14.50%, 14.25% 14%, 13.75%, 13.50%, 13.25%, 13%, 12.75%, 12.50%, 12.25%, 12%, 11.75%, 11.50%, 11.25% 11%, 10.75%, 10.50%, 10.25% 10%, 9.75%, 9.50%, 9.25%, 9%, 8.75%, 8.50%, 8.25% 8%, 7.75%, 7.50%, 7.25%, 7%, 6.75%, 6.50%, 6.25%, 6%, 5.75%, 5.50%, 5.25%, 5%, 4.75%, 4.50%, 4.25%, 4%, 3.75%, 3.50%, 3.25%, 3%, 2.75%, 2.50%, 2.25%, 2%, 1.75%, 1.50%, 1.25%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% (or a number in the range defined by and including any two numbers above) w/w, w/v, or v/v.

In some embodiments, the concentration of one or more compounds of the invention is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v.

In some embodiments, the concentration of one or more compounds of the invention is in the range from approximately 0.001 % to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

In some embodiments, the amount of one or more compounds of the invention is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g (or a number in the range defined by and including any two numbers above).

In some embodiments, the amount of one or more compounds of the invention is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g,, 0.15 g, 0.2 g,, 0.25 g, 0.3 g,, 0.35 g, 0.4 g,, 0.45 g, 0.5 g, 0.55 g, 0.6 g,, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g (or a number in the range defined by and including any two numbers above).

In some embodiments, the amount of one or more compounds of the invention is in the range of 0.0001-10 g, 0.0005-9 g, 0.001-8 g, 0.005-7 g, 0.01-6 g, 0.05-5 g, 0.1-4 g, 0.5-4 g, or 1-3 g.

The compounds according to the invention are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. An exemplary dosage is 10 to 30 mg per day. The exact dosage will depend upon the route of administration, the form in which the compound is administered, the subject to be treated, the body weight of the subject to be treated, and the preference and experience of the attending physician.

A pharmaceutical composition of the invention typically contains an active ingredient (i.e., a compound of the disclosure) of the present invention or a pharmaceutically acceptable salt and/or coordination complex thereof, and one or more pharmaceutically acceptable excipients, carriers, including but not limited to inert solid diluents and fillers, diluents, sterile aqueous solution and various organic solvents, permeation enhancers, solubilizers and adjuvants.

Described below are non- limiting exemplary pharmaceutical compositions and methods for preparing the same.

### Pharmaceutical Compositions for Oral Administration

In some embodiments, the invention provides a pharmaceutical composition for oral administration containing a compound of the invention, and a pharmaceutical excipient suitable for oral administration.

In some embodiments, the invention provides a solid pharmaceutical composition for oral administration containing: (i) an effective amount of a compound of the invention; optionally (ii) an effective amount of a second agent; and (iii) a pharmaceutical excipient suitable for oral administration. In some embodiments, the composition further contains: (iv) an effective amount of a third agent.

In some embodiments, the pharmaceutical composition may be a liquid pharmaceutical composition suitable for oral consumption. Pharmaceutical compositions of the invention suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in- water emulsion, or a water-in-oil liquid emulsion. Such dosage forms can be prepared by any of the methods of pharmacy, but all methods include the step of bringing the active ingredient into association with the carrier, which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free- flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

This invention further encompasses anhydrous pharmaceutical compositions and dosage forms comprising an active ingredient, since water can facilitate the degradation of some compounds. For example, water may be added (e.g., 5%) in the pharmaceutical arts as a means of simulating long-term storage in order to determine characteristics such as shelf- life or the stability of formulations over time. Anhydrous pharmaceutical compositions and dosage forms of the invention can be prepared using anhydrous or low moisture containing ingredients and low moisture or low humidity conditions. Pharmaceutical compositions and dosage forms of the invention which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

An active ingredient can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the invention to provide tablets that disintegrate when exposed to an aqueous environment. Too much of a disintegrant may produce tablets which may disintegrate in the bottle. Too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

When aqueous suspensions and/or elixirs are desired for oral administration, the active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactant which can be used to form pharmaceutical compositions and dosage forms of the invention include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions.

Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof, camitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP -phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but are not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG- 10 laurate, PEG- 12 laurate, PEG-20 laurate, PEG-32 laurate, PEG-32 dilaurate, PEG- 12 oleate, PEG- 15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG- 100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG- 100 succinate, PEG-24 cholesterol, polyglyceryl-lOoleate, Tween 40, Tween 60, sucrose monostearate, sucrose mono laurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In one embodiment, the composition may include a solubilizer to ensure good solubilization and/or dissolution of the compound of the present invention and to minimize precipitation of the compound of the present invention. This can be especially important for compositions for non-oral use, e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG ; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a subject using conventional techniques, such as distillation or evaporation. Thus, if present, the solubilizer can be in a weight ratio of 10%, 25%o, 50%), 100%o, or up to about 200%> by weight, based on the combined weight of the drug, and other excipients. If desired, very small amounts of solubilizer may also be used, such as 5%>, 2%>, 1%) or even less. Typically, the solubilizer may be present in an amount of about 1%> to about 100%, more typically about 5%> to about 25%> by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

### Pharmaceutical Compositions for Injection

In some embodiments, the invention provides a pharmaceutical composition for injection containing a compound of the present invention and a pharmaceutical excipient suitable for injection. Components and amounts of agents in the compositions are as described herein.

The forms in which the novel compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the compound of the present invention in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze- drying techniques which yield a powder of the active ingredient plus any additional desired ingredient from a previously sterile-filtered solution thereof.

### Pharmaceutical Compositions for Topical (e.g. Transdermal) Delivery

In some embodiments, the invention provides a pharmaceutical composition for transdermal delivery containing a compound of the present invention and a pharmaceutical excipient suitable for transdermal delivery.

Compositions of the present invention can be formulated into preparations in solid, semisolid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration- enhancing molecules known to those trained in the art of topical formulation.

Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present invention employs transdermal delivery devices (“patches”). Such transdermal patches may be used to provide continuous or discontinuous infusion of a compound of the present invention in controlled amounts, either with or without another agent.

The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

### Pharmaceutical Compositions for Inhalation

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner.

### Other Pharmaceutical Compositions

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001 ; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999); all of which are incorporated by reference herein in their entirety.

Administration of the compounds or pharmaceutical composition of the present invention can be effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g. transdermal application), rectal administration, via local delivery by catheter or stent or through inhalation. Compounds can also be administered intraadiposally or intrathecally.

In some embodiments, the compounds or pharmaceutical composition of the present invention are administered by intravenous injection.

The amount of the compound administered will be dependent on the subject being treated, the severity of the disorder or condition, the rate of administration, the disposition of the compound and the discretion of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to 7 g/day, preferably about 0.05 to about 2.5 g/day. In some instances, dosage levels below the lower limit of the aforesaid range may be more than adequate, while in other cases still larger doses may be employed without causing any harmful side effect, e.g. by dividing such larger doses into several small doses for administration throughout the day.

In some embodiments, a compound of the invention is administered in a single dose.

Typically, such administration will be by injection, e.g., intravenous injection, in order to introduce the agent quickly. However, other routes may be used as appropriate. A single dose of a compound of the invention may also be used for treatment of an acute condition.

In some embodiments, a compound of the invention is administered in multiple doses. Dosing may be about once, twice, three times, four times, five times, six times, or more than six times per day. Dosing may be about once a month, once every two weeks, once a week, or once every other day. In another embodiment a compound of the invention and another agent are administered together about once per day to about 6 times per day. In another embodiment the administration of a compound of the invention and an agent continues for less than about 7 days. In yet another embodiment the administration continues for more than about 6, 10, 14, 28 days, two months, six months, or one year. In some cases, continuous dosing is achieved and maintained as long as necessary.

Administration of the compounds of the invention may continue as long as necessary. In some embodiments, a compound of the invention is administered for more than 1, 2, 3, 4, 5, 6, 7, 14, or 28 days. In some embodiments, a compound of the invention is administered for less than 28, 14, 7, 6, 5, 4, 3, 2, or 1 day. In some embodiments, a compound of the invention is administered chronically on an ongoing basis, e.g., for the treatment of chronic effects.

An effective amount of a compound of the invention may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

The compositions of the invention may also be delivered via an impregnated or coated device such as a stent, for example, or an artery-inserted cylindrical polymer. Such a method of administration may, for example, aid in the prevention or amelioration of restenosis following procedures such as balloon angioplasty. Without being bound by theory, compounds of the invention may slow or inhibit the migration and proliferation of smooth muscle cells in the arterial wall which contribute to restenosis. A compound of the invention may be administered, for example, by local delivery from the struts of a stent, from a stent graft, from grafts, or from the cover or sheath of a stent. In some embodiments, a compound of the invention is admixed with a matrix. Such a matrix may be a polymeric matrix, and may serve to bond the compound to the stent. Polymeric matrices suitable for such use, include, for example, lactone-based polyesters or copolyesters such as polylactide, polycaprolactonglycolide, polyorthoesters, polyanhydrides, polyaminoacids, polysaccharides, polyphosphazenes, poly (ether-ester) copolymers (e.g. PEO-PLLA); polydimethylsiloxane, poly(ethylene-vinylacetate), acrylate-based polymers or copolymers (e.g. polyhydroxyethyl methylmethacrylate, polyvinyl pyrrolidinone), fluorinated polymers such as polytetrafluoroethylene and cellulose esters. Suitable matrices may be nondegrading or may degrade with time, releasing the compound or compounds. Compounds of the invention may be applied to the surface of the stent by various methods such as dip/spin coating, spray coating, dip-coating, and/or brush-coating. The compounds may be applied in a solvent and the solvent may be allowed to evaporate, thus forming a layer of compound onto the stent. Alternatively, the compound may be located in the body of the stent or graft, for example in microchannels or micropores. When implanted, the compound diffuses out of the body of the stent to contact the arterial wall. Such stents may be prepared by dipping a stent manufactured to contain such micropores or microchannels into a solution of the compound of the invention in a suitable solvent, followed by evaporation of the solvent. Excess drug on the surface of the stent may be removed via an additional brief solvent wash. In yet other embodiments, compounds of the invention may be covalently linked to a stent or graft. A covalent linker may be used which degrades in vivo, leading to the release of the compound of the invention. Any bio-labile linkage may be used for such a purpose, such as ester, amide or anhydride linkages. Compounds of the invention may additionally be administered intravascularly from a balloon used during angioplasty. Extravascular administration of the compounds via the pericard or via advential application of formulations of the invention may also be performed to decrease restenosis.

A variety of stent devices which may be used as described are disclosed, for example, in the following references, all of which are hereby incorporated by reference: U.S. Pat. No. 5451233; U.S. Pat. No. 5040548; U.S. Pat. No. 5061273; U.S. Pat. No. 5496346; U.S. Pat. No. 5292331; U.S. Pat. No. 5674278; U.S. Pat. No. 3657744; U.S. Pat. No. 4739762; U.S. Pat. No. 5195984; U.S. Pat. No. 5292331 ; U.S. Pat. No. 5674278; U.S. Pat. No. 5879382; U.S. Pat. No. 6344053.

The compounds of the invention may be administered in dosages. It is known in the art that due to intersubject variability in compound pharmacokinetics, individualization of dosing regimen is necessary for optimal therapy. Dosing for a compound of the invention may be found by routine experimentation in light of the instant disclosure.

When a compound of the invention is administered in a composition that comprises one or more agents, and the agent has a shorter half- life than the compound of the invention unit dose forms of the agent and the compound of the invention may be adjusted accordingly.

The subject pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compound in sterile aqueous solutions, for example, aqueous propylene glycol or dextrose solutions. Such dosage forms can be suitably buffered, if desired.

### Methods of Use

The method typically comprises administering to a subject a therapeutically effective amount of a compound of the invention. The therapeutically effective amount of the subject combination of compounds may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of proliferation or downregulation of activity of a target protein. The specific dose will vary depending on the particular compounds chosen, the dosing regimen to be followed, whether it is administered in combination with other compounds, timing of administration, the tissue to which it is administered, and the physical delivery system in which it is carried.

As used herein, the term “IC₅₀” refers to the half maximal inhibitory concentration of an inhibitor in inhibiting biological or biochemical function. This quantitative measure indicates how much of a particular inhibitor is needed to inhibit a given biological process (or component of a process, i.e. an enzyme, cell, cell receptor or microorganism) by half. In other words, it is the half maximal (50%) inhibitory concentration (IC) of a substance (50% IC, or IC50). EC50 refers to the plasma concentration required for obtaining 50%> of a maximum effect in vivo.

In some embodiments, the subject methods utilize a MCL-1 inhibitor with an IC50 value of about or less than a predetermined value, as ascertained in an in vitro assay. In some embodiments, the MCL-1 inhibitor inhibits MCL-1 a with an IC50 value of about 1 nM or less, 2 nM or less, 5 nM or less, 7 nM or less, 10 nM or less, 20 nM or less, 30 nM or less, 40 nM or less, 50 nM or less, 60 nM or less, 70 nM or less, 80 nM or less, 90 nM or less, 100 nM or less, 120 nM or less, 140 nM or less, 150 nM or less, 160 nM or less, 170 nM or less, 180 nM or less, 190 nM or less, 200 nM or less, 225 nM or less, 250 nM or less, 275 nM or less, 300 nM or less, 325 nM or less, 350 nM or less, 375 nM or less, 400 nM or less, 425 nM or less, 450 nM or less, 475 nM or less, 500 nM or less, 550 nM or less, 600 nM or less, 650 nM or less, 700 nM or less, 750 nM or less, 800 nM or less, 850 nM or less, 900 nM or less, 950 nM or less, 1 µM or less, 1.1 µM or less, 1.2 µM or less, 1.3 µM or less, 1.4 µM or less, 1.5 µM or less, 1.6 µM or less, 1.7 µM or less, 1.8 µM or less, 1.9 µM or less, 2 µM or less, 5 µM or less, 10 µM or less, 15 µM or less, 20 µM or less, 25 µM or less, 30 µM or less, 40 µM or less, 50 µM, 60 µM, 70 µM, 80 µM, 90 µM, 100 µM, 200 µM, 300 µM, 400 µM, or 500 µM, or less, (or a number in the range defined by and including any two numbers above).

In some embodiments, the MCL-1 inhibitor selectively inhibits MCL-1 a with an IC50 value that is at least 2, 3, 4, 5, 6, 7, 8, 9, 10, 15, 20, 25, 30, 35, 40, 45, 50, 100, or 1000 times less (or a number in the range defined by and including any two numbers above)than its IC50 value against one, two, or three other MCL-1s.

In some embodiments, the MCL-1 inhibitor selectively inhibits MCL-1 a with an IC50 value that is less than about 1 nM, 2 nM, 5 nM, 7 nM, 10 nM, 20 nM, 30 nM, 40 nM, 50 nM, 60 nM, 70 nM, 80 nM, 90 nM, 100 nM, 120 nM, 140 nM, 150 nM, 160 nM, 170 nM, 180 nM, 190 nM, 200 nM, 225 nM, 250 nM, 275 nM, 300 nM, 325 nM, 350 nM, 375 nM, 400 nM, 425 nM, 450 nM, 475 nM, 500 nM, 550 nM, 600 nM, 650 nM, 700 nM, 750 nM, 800 nM, 850 nM, 900 nM, 950 nM, 1 µM, 1.1 µM, 1.2 µM, 1.3 µM, 1.4 µM, 1.5 µM, 1.6 µM, 1.7 µM, 1.8 µM, 1.9 µM, 2 µM, 5 µM, 10 µM, 15 µM, 20 µM, 25 µM, 30 µM, 40 µM, 50 µM, 60 µM, 70 µM, 80 µM, 90 µM, 100 µM, 200 µM, 300 µM, 400 µM, or 500 µM (or in the range defined by and including any two numbers above), and said IC50 value is at least 2, 3, 4, 5, 6, 7, 8, 9, 10, 15, 20, 25, 30, 35, 40, 45, 50, 100, or 1000 times less (or a number in the range defined by and including any two numbers above) than its IC50 value against one, two or three other MCL-1s.

The subject methods are useful for treating a disease condition associated with MCL-1. Any disease condition that results directly or indirectly from an abnormal activity or expression level of MCL-1 can be an intended disease condition.

Different disease conditions associated with MCL-1 have been reported. MCL-1 has been implicated, for example, auto-immune diseases, neurodegeneration (such as Parkinson’s disease, Alzheimer’s disease and ischaemia), inflammatory diseases, viral infections and cancer such as, for example, colon cancer, breast cancer, small-cell lung cancer, non-small-cell lung cancer, bladder cancer, ovarian cancer, prostate cancer, chronic lymphoid leukemia, lymphoma, myeloma, acute myeloid leukemia, or pancreatic cancer.

Non- limiting examples of such conditions include but are not limited to Acanthoma, Acinic cell carcinoma, Acoustic neuroma, Acral lentiginous melanoma, Acrospiroma, Acute eosinophilic leukemia, Acute lymphoblastic leukemia, Acute lymphocytic leukemia, Acute megakaryoblastic leukemia, Acute monocytic leukemia, Acute myeloblasts leukemia with maturation, Acute myeloid dendritic cell leukemia, Acute myeloid leukemia, Acute myelogenous leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt’s lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman’s Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epidermoid cancer, Epithelioid sarcoma, Erythroleukemia, Esophageal cancer, Esthesioneuroblastoma, Ewing Family of Tumor, Ewing Family Sarcoma, Ewing’s sarcoma, Extracranial Germ Cell Tumor, Extragonadal Germ Cell Tumor, Extrahepatic Bile Duct Cancer, Extramammary Paget’s disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Head and Neck Cancer, Head and neck cancer, Heart cancer, Hemoglobinopathies such as b-thalassemia and sickle cell disease (SCD), Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin’s lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi’s sarcoma, Kidney Cancer, Klatskin tumor, Krukenberg tumor, Laryngeal Cancer, Laryngeal cancer, Lentigo maligna melanoma, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mastocytosis, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplasia Disease, Myelodysplasia Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget’s disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene onChromosome 15, Retinoblastoma, Rhabdomyoma, Rhabdomyosarcoma, Richter’s transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom’s macroglobulinemia, Warthin’s tumor, Wilms’ tumor, or any combination thereof.

In some embodiments, said method is for treating a disease selected from the group consisting of tumor angiogenesis, chronic inflammatory disease such as rheumatoid arthritis, atherosclerosis, inflammatory bowel disease, skin diseases such as psoriasis, eczema, and scleroderma, diabetes, diabetic retinopathy, retinopathy of prematurity, age-related macular degeneration, hemangioma, glioma, melanoma, Kaposi’s sarcoma and ovarian, breast, lung, pancreatic, prostate, colon and epidermoid cancer.

In other embodiments, said method is for treating a disease selected from breast cancer, lung cancer, pancreatic cancer, prostate cancer, colon cancer, ovarian cancer, uterine cancer, or cervical cancer.

In other embodiments, said method is for treating a disease selected from leukemia such as acute myeloid leukemia (AML), acute lymphocytic leukemia, chronic lymphocytic leukemia, chronic myeloid leukemia, hairy cell leukemia, myelodysplasia, myeloproliferative disorders, acute myelogenous leukemia (AML), chronic myelogenous leukemia (CML), mastocytosis, chronic lymphocytic leukemia (CLL), multiple myeloma (MM), myelodysplastic syndrome (MDS) or epidermoid cancer.

Compounds of the disclosure, as well as pharmaceutical compositions comprising them, can be administered to treat any of the described diseases, alone or in combination with a medical therapy. Medical therapies include, for example, surgery and radiotherapy (e.g., gamma-radiation, neutron beam radiotherapy, electron beam radiotherapy, proton therapy, brachytherapy, systemic radioactive isotopes).

In other aspects, compounds of the disclosure, as well as pharmaceutical compositions comprising them, can be administered to treat any of the described diseases, alone or in combination with one or more other agents.

In other methods, the compounds of the disclosure, as well as pharmaceutical compositions comprising them, can be administered in combination with agonists of nuclear receptors agents.

In other methods, the compounds of the disclosure, as well as pharmaceutical compositions comprising them, can be administered in combination with antagonists of nuclear receptors agents.

In other methods, the compounds of the disclosure, as well as pharmaceutical compositions comprising them, can be administered in combination with an anti-proliferative agent.

### Combination Therapies

For treating cancer and other proliferative diseases, the compounds of the invention can be used in combination with chemotherapeutic agents, agonists or antagonists of nuclear receptors, or other anti-proliferative agents. The compounds of the invention can also be used in combination with a medical therapy such as surgery or radiotherapy, e.g., gamma-radiation, neutron beam radiotherapy, electron beam radiotherapy, proton therapy, brachytherapy, and systemic radioactive isotopes. Examples of suitable chemotherapeutic agents include any of: abarelix, aldesleukin, alemtuzumab, alitretinoin, allopurinol, all-trans retinoic acid, altretamine, anastrozole, arsenic trioxide, asparaginase, azacitidine, bendamustine, bevacizumab, bexarotene, bleomycin, bortezombi, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panobinostat, panitumumab, pegaspargase, pegfilgrastim, pemetrexed disodium, pentostatin, pipobroman, plicamycin, procarbazine, quinacrine, rasburicase, rituximab, ruxolitinib, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinstat and zoledronate.

In some embodiments, the compounds of the invention can be used in combination with a therapeutic agent that targets an epigenetic regulator. Examples of epigenetic regulators include bromodomain inhibitors, the histone lysine methyltransferase inhibitors, histone arginine methyl transferase inhibitors, histone demethylase inhibitors, histone deacetylase inhibitors, histone acetylase inhibitors, and DNA methyltransferase inhibitors. Histone deacetylase inhibitors include, e.g., vorinostat. Histone arginine methyl transferase inhibitors include inhibitors of protein arginine methyltransferases (PRMTs) such as PRMT5, PRMT1 and PRMT4. DNA methyltransferase inhibitors include inhibitors of DNMT1 and DNMT3.

For treating cancer and other proliferative diseases, the compounds of the invention can be used in combination with targeted therapies, including JAK kinase inhibitors (e.g. Ruxolitinib), PI3 kinase inhibitors including PI3K-delta selective and broad spectrum PI3K inhibitors, MEK inhibitors, Cyclin Dependent kinase inhibitors, including CDK4/6 inhibitors and CDK9 inhibitors, BRAF inhibitors, mTOR inhibitors, proteasome inhibitors (e.g. Bortezomib, Carfilzomib), HDAC inhibitors (e.g. panobinostat, vorinostat), DNA methyl transferase inhibitors, dexamethasone, bromo and extra terminal family member (BET) inhibitors, BTK inhibitors (e.g. ibrutinib, acalabrutinib), BCL2 inhibitors (e.g. venetoclax), dual BCL2 family inhibitors (e.g. BCL2/BCLxL), PARP inhibitors, FLT3 inhibitors, or LSD1 inhibitors.

In some embodiments, the inhibitor of an immune checkpoint molecule is an inhibitor of PD-1, e.g., an anti-PD-1 monoclonal antibody. In some embodiments, the anti-PD-1 monoclonal antibody is nivolumab, pembrolizumab (also known as MK-3475), or PDR001. In some embodiments, the anti-PD-1 monoclonal antibody is nivolumab or pembrolizumab. In some embodiments, the anti-PD1 antibody is pembrolizumab. In some embodiments, the inhibitor of an immune checkpoint molecule is an inhibitor of PD-L1, e.g., an anti-PD-L1 monoclonal antibody. In some embodiments, the anti-PD-L1 monoclonal antibody is atezolizumab, durvalumab, or BMS-935559. In some embodiments, the inhibitor of an immune checkpoint molecule is an inhibitor of CTLA-4, e.g., an anti-CTLA-4 antibody. In some embodiments, the anti-CTLA-4 antibody is ipilimumab.

In some embodiments, the agent is an alkylating agent, a proteasome inhibitor, a corticosteroid, or an immunomodulatory agent. Examples of an alkylating agent include cyclophosphamide (CY), melphalan (MEL), and bendamustine. In some embodiments, the proteasome inhibitor is carfilzomib. In some embodiments, the corticosteroid is dexamethasone (DEX). In some embodiments, the immunomodulatory agent is lenalidomide (LEN) or pomalidomide (POM).

For treating autoimmune or inflammatory conditions, the compound of the invention can be administered in combination with a corticosteroid such as triamcinolone, dexamethasone, fluocinolone, cortisone, prednisolone, or flumetholone.

For treating autoimmune or inflammatory conditions, the compound of the invention can be administered in combination with an immune suppressant such as fluocinolone acetonide (Retisert®), rimexolone (AL-2178, Vexol, Alcon), or cyclosporine (Restasis®).

### Synthesis

Compounds of the invention can be prepared according to numerous preparatory routes known in the literature. The Scheme below provide general guidance in connection with preparing the compounds of the invention. One skilled in the art would understand that the preparations shown in the Scheme can be modified or optimized using general knowledge of organic chemistry to prepare various compounds of the invention. Example synthetic methods for preparing compounds of the invention are provided in the Scheme below.

### Intermediate 1

### 6′-Chlorospiro[4,5-dihydro-2H-1,5-benzoxazepine-3,1′-tetralin]-7-sulfonamide

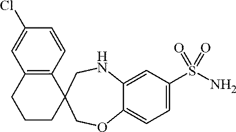 [SMILES: NS(=O)(=O)c1ccc2c(c1)NCC1(CCCc3cc(Cl)ccc31)CO2]

### Step 1: 6′-chlorospiro{oxirane-2,1′-tetralin]

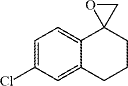 [SMILES: Clc1ccc2c(c1)CCCC21CO1]

To a solution of 6-chlorotetralin-1-one (10.0 g, 55.3 mmol) in DMSO (100 mL) was added trimethylsulfonium iodide (12.4 g, 60.9 mmol) and hydroxypotassium (6.21 g, 110 mmol), the mixture was stirred at 25° C. for 24 hours. The mixture was added to ice water (500 mL), extracted with MTBE (400 mL X 3), combined the organic phases, washed with brine (500 mL × 2), dried over Na₂SO₄, filtered and concentrated in vacuum to give 6′-chlorospiro[oxirane-2,1′-tetralin] (10.0 g, 51.3 mmol, 92% yield).

### Step 2: 6-chlorotetralin-1-carbaldehyde

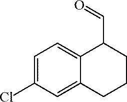 [SMILES: O=CC1CCCc2cc(Cl)ccc21]

To a solution of 6′-chlorospiro[oxirane-2,1′-tetralin] (10.0 g, 51.3 mmol) in THF (160 mL) was added Boron trifluoride etherate (364 mg, 2.57 mmol) at -8° C., the solution was stirred at -8° C. for 10 mins. The reaction was quenched with sat. NaHCO₃ (200 mL) at -8° C., extracted the aqueous with MTBE (400 mL × 2), combined the organic phases, washed with brine (400 mL), dried over Na₂SO₄, filtered and concentrated in vacuum to give 6-chlorotetralin-1-carbaldehyde (11.40 g, 70% purity, 40.995 mmol, 79% yield).

### Step 3: [6-chloro-1-(hydroxymethyl)tetralin-1-yl]methanol

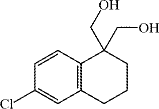 [SMILES: OCC1(CO)CCCc2cc(Cl)ccc21]

To a solution of 6-chlorotetralin-1-carbaldehyde (11.4 g, 70% purity, 41 mmol) in 2-(2-hydroxyethoxy)ethanol (80 mL, 41 mmol) was added paraformaldehyde (56 mL, 41 mmol), then potassium hydroxide (56 mL, 41 mmol) was added to the mixture at 5° C. The reaction mixture was stirred at 45° C. for 1 h.. Thereaction mixture was added brine (250 mL), extracted with DCM (300 mL × 3), combined the organic phases, dried over Na₂SO₄, filtered and concentrated in vacuum, the residue was purified by silica gel column chromatography (PE : EA = 1.5 : 1) to give [6-chloro-1-(hydroxymethyl)tetralin-1-yl]methanol (11.2 g, 75% purity, 90% yield).¹H NMR (400 MHz, CDCl₃): δ 7.31-7.34 (m, 2 H), 7.11-7.14 (m, 2 H), 3.87-3.91 (m, 2 H), 3.72-3.76 (m, 2 H), 2.73-2.76 (m, 2 H), 2.11-2.15 (m, 2 H), 1.89-1.92 (m, 2 H), 1.79-1.83 (m, 2 H).

### Step 4: 6-chloro-1-(hydroxymethyl)tetralin-1-yl]methyl Benzoate

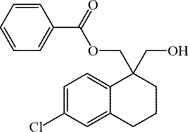 [SMILES: O=C(OCC1(CO)CCCc2cc(Cl)ccc21)c1ccccc1]

To a solution of [6-chloro-1-(hydroxymethyl)tetralin-1-yl]methanol (11.2 g, 37 mmol) in DCM (150 mL) was added benzoyl chloride (6.26 g, 44 mmol) at 0° C., following by drop-wise addition of DIPEA (7.4 mL, 44 mmol). The mixture stirred at 25° C. 16 h.. Added DCM (150 mL) to the mixture, washed with sat. NH₄Cl (100 mL) and brine (100 mL), dried over Na₂SO₄, filtered and concentrated in vacuum, the residue was purified by silica gel column chromatography (PE : EA = 9 : 1) to give 11.65 g of racemic product. ¹H NMR (400 MHz, CDCl₃): δ 8.00-8.02 (m, 2 H), 7.57-7.61 (m, 1 H), 7.44-7.48 (m, 3 H), 7.14-7.16(m, 2 H), 4.48 (s, 2 H), 3.74-3.82 (m, 2 H), 2.78-2.81 (m, 2 H), 1.83-1.95 (m, 4 H).

### Step 5: (6-chloro-1-formyl-tetralin-1-yl)methyl Benzoate

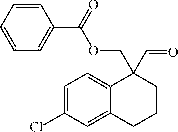 [SMILES: O=CC1(COC(=O)c2ccccc2)CCCc2cc(Cl)ccc21]

To a solution of [6-chloro-1-(hydroxymethyl)tetralin-1-yl]methyl benzoate (1.48 g, 4.47 mmol) in DCM (25 mL) was added Dess-Martin periodinane (2.84 g, 6.7 mmol) at 0° C., then the mixture was stirred at 25° C. for 1 h. To the reaction mixture was added a 1:1 mixture of 10% Na₂S₂O₃/sat. NaHCO₃ solution (100 mL). The mixture was extracted with DCM (100 mL × 2). The combined organic phases were washed with brine (15 mL), dried over Na₂SO₄, filtered and concentrated under reduced pressure. The residue was purified by FC on a silica gel column to give (6-chloro-1-formyl-tetralin-1-yl)methyl benzoate (1.24 g, 84% yield). ¹H NMR (400 MHz, CDCl₃): δ 9.61 (s, 1 H), 7.94-7.96 (m, 2 H), 7.54-7.58 (m, 1 H), 7.41-7.45 (m, 2 H), 7.15-7.21 (m, 3 H), 4.75 (d, J = 11.6 Hz, 1H), 4.55 (d, J = 11.6 Hz, 1H), 2.81-2.85 (m, 2 H), 2.19-2.23 (m, 1 H), 2.00-2.06 (m, 1 H), 1.89-1.95 (m, 2 H).

### Step 6: [6-chloro-1-(dimethoxymethyl)tetralin-1-yl]methanol

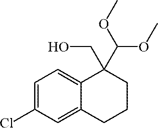 [SMILES: COC(OC)C1(CO)CCCc2cc(Cl)ccc21]

To a solution of (6-chloro-1-formyl-tetralin-1-yl)methyl benzoate (1.24 g, 3.77 mmol) in methanol (25 mL) were added p-TsOH H₂O (35 mg, 0.19 mmol) and trimethyl orthoformate (1.2 g, 11.3 mmol). The mixture was stirred at 70° C. for 4 h., then concentrated to 50% volume. The residue was diluted with THF (25 mL) and 1 N NaOH (25 mL) was added. The resulting reaction mixture was stirred at 40° C. 4 h.The solvent was removed. The residue was extracted with EA (20 mL × 3). The combined organic layers were washed with 1 N NaOH (50 mL) and brine (100 mL), dried over Na₂SO₄, and concentrated under vacuum. The residue was purified by FC on a silica gel column (PE:EA=9:1) to give [6-chloro-1-(dimethoxymethyl)tetralin-1-yl]methanol (0.98 g, 96% yield). ¹H NMR (400 MHz, CDCl₃): δ 7.35 (d, J = 8.4 Hz, 1 H), 7.10-7.13 (m, 2 H), 4.49 (s, 1 H), 3.90 (dd, J = 3.8, 11.2 Hz, 1 H), 3.53 (dd, J = 8.4, 11.2 Hz, 1 H), 3.46 (s, 3 H), 3.33 (s, 3 H), 2.68-2.76 (m, 2 H), 1.99-2.06 (m, 1 H), 1.89-1.96 (m, 1 H), 1.70-1.86 (m, 2 H).

### Step 7: 4-[[6-chloro-1-(dimethoxymethyl)tetralin-1-yl]methoxy]-3-nitro-benzenesulfonamide

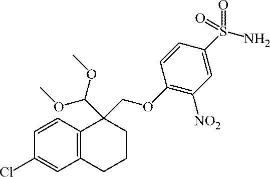 [SMILES: COC(OC)C1(COc2ccc(S(N)(=O)=O)cc2[N+](=O)[O-])CCCc2cc(Cl)ccc21]

A 100 mL flask with septum containing a mixture of [6-chloro-1-(dimethoxymethyl)tetralin-1-yl]methanol (818 mg, 3.02 mmol) and potassium t-butoxide (779 mg, 6.94 mmol) under N₂ was charged with THF (22 mL) giving a tan solution. The solution was stirred for 5 min at 0° C., followed by addition at 0° C. of a solution of 4-Fluoro-3-nitrobenzenesulfonamide (731 mg, 3.32 mmol) in THF (4 mL) over 8 min. The reaction was stirred at 0° C. for 20 min. The reaction mixture was quenched with sat. NH₄Cl (10 mL).The reaction mixture was diluted with water (80 mL) and sat. NH₄Cl (10 mL), and extracted with EtOAc (100 mL). The organic layer was washed with water (70 mL) and sat. NH₄Cl (10 mL), and brine (50 mL). The aqueous layers were combined, and back-extracted with EtOAc (60 mL), washed with water (60 mL), and brine (30 mL). The organic layers were combined, dried over Na₂SO₄, and filtered and concentrated under reduced pressure to afford 4-[[6-chloro-1-(dimethoxymethyl)tetralin-1-yl]methoxy]-3-nitro-benzenesulfonamide as a yellow foam (1.52 g) and was used directly in the next reaction without further purification. R_{f} = 0.36 (1:1 hexanes:EtOAc); ¹H NMR (500 MHz, DMSO-d₆) δ 8.28 (d, J= 2.3 Hz, 1H), 8.01 (dd, J= 2.4, 8.9 Hz, 1H), 7.60 (dd, J= 8.7, 16.3 Hz, 2H), 7.50 (s, 2H), 7.19 - 7.11 (m, 2H), 4.63 (s, 1H), 4.38 - 4.26 (m, 2H), 3.38 (s, 3H), 3.29 (s, 3H), 2.70 (d, J= 6.2 Hz, 2H), 2.04 - 1.94 (m, 1H), 1.90 -1.79 (m, 2H), 1.77 - 1.67 (m, 1H).

### Step 8: 4-[(6-chloro-1-formyl-tetralin-1-yl)methoxy [-3-nitro-benzenesulfonamide

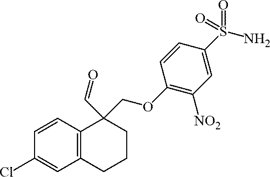 [SMILES: NS(=O)(=O)c1ccc(OCC2(C=O)CCCc3cc(Cl)ccc32)c([N+](=O)[O-])c1]

The Amberlyst 16 wet catalyst was rinsed with acetone and dried under high vacuum before use. A 500 mL RBF with septum containing crude 4-[[6-chloro-1-(dimethoxymethyl)tetralin-1-yl]methoxy]-3-nitro-benzenesulfonamide (1.42 g, 3.02 mmol) and pre-treated Amberlyst 16 wet (1 g, ~7.44 mmol) under N₂ was charged with acetone (30 mL). The reaction mixture was heated at 50° C. for 2 h., filtered through cotton and rinsed with DCM. The filtrate was concentrated under reduced pressure to afford 4-[(6-chloro-1-formyl-tetralin-1-yl)methoxy]-3-nitro-benzenesulfonamide as an orange/brown oil (1.7 g) which was used directly in the next reaction without further purification. R_{f} = 0.31 (1:1 hexanes:EtOAc); ¹H NMR (500 MHz, DMSO-d₆) δ 9.65 (s, 1H), 8.27 (d, J= 2.4 Hz, 1H), 8.03 (dd, J= 2.4, 8.9 Hz, 1H), 7.63 (d, J= 9.0 Hz, 1H), 7.50 (s, 2H), 7.35 - 7.29 (m, 2H), 7.26 (dd, J= 2.4, 8.4 Hz, 1H), 4.77 (d, J= 9.6 Hz, 1H), 4.47 (d, J= 9.6 Hz, 1H), 2.78 (t, J= 6.3 Hz, 2H), 2.19 (ddd, J= 3.0, 8.9, 13.2 Hz, 1H), 1.99 (ddd, J= 2.8, 8.1, 13.5 Hz, 1H), 1.89 - 1.80 (m, 1H), 1.80 - 1.70 (m, 1H).

### Step 9: 6′-chlorospiro[4,5-dihydro-2H-1,5-benzoxazepine-3,1′-tetralin]-7-sulfonamide

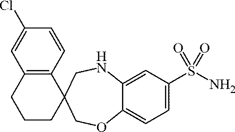 [SMILES: NS(=O)(=O)c1ccc2c(c1)NCC1(CCCc3cc(Cl)ccc31)CO2]

A solution of crude 4-[(6-chloro-1-formyl-tetralin-1-yl)methoxy]-3-nitro-benzenesulfonamide (assumed 3.02 mmol) in acetic acid (50 mL) was charged with iron powder (1.69 g, 30.2 mmol). The mixture was heated at 70° C. for 3 h. The mixture was charged with Celite, diluted with DCM (50 mL), filtered through a Celite plug, and rinsed with DCM to yield crude 6′-chlorospiro[2H-1,5-benzoxazepine-3,1′-tetralin]-7-sulfonamide. R_{f} = 0.24 (1:1 EtOAc/hexanes); LCMS calculated for C₁₈H₁₈ClN₂O₃S (M+H)⁺: m/z = 377.07/379.07; found: 377.0/379.0.

The filtrate was concentrated under reduced pressure, dissolved in DCM (30 mL), cooled to 0° C., and charged with sodium triacetoxyborohydride (1.99 g, 9.44 mmol) over 1 min. The reaction mixture was stirred at 0° C. for 1 min, then stirred at RT for 80 min. The reaction mixture was quenched with 10% citric acid (30 mL), diluted with water (30 mL), and extracted with EtOAc (125 mL). The organic layer was washed with 10% citric acid (10 mL) and water (40 mL), washed with brine (2× 40 mL), dried over Na₂SO₄, and filtered. The filtrate was concentrated under reduced pressure to yield 6′-chlorospiro[4,5-dihydro-2H-1,5-benzoxazepine-3,1′-tetralin]-7-sulfonamide (1.24 g, 2.61 mmol, 86% yield) as a light tan foam.R_{f} = 0.45 (1:1 EtOAc/hexanes). LCMS calculated for C₁₈H₂₀ClN₂O₃S (M+H)⁺: m/z = 379.09/379.08; found: 379.0/381.0; ¹H NMR (500 MHz, DMSO-d₆) δ 7.81 (d, J= 8.5 Hz, 1H), 7.26 (dd, J= 2.4, 8.5 Hz, 1H), 7.18 (dd, J= 2.3, 15.2 Hz, 2H), 7.13 (s, 2H), 7.02 (dd, J= 2.3, 8.4 Hz, 1H), 6.92 (d, J= 8.4 Hz, 1H), 6.20 (t, J= 4.1 Hz, 1H), 4.08 (q, J= 12.2 Hz, 2H), 3.23 (dd, J= 4.7, 13.7 Hz, 1H), 2.77 - 2.65 (m, 2H), 1.87 - 1.66 (m, 3H), 1.55 (ddd, J= 2.9, 9.7, 12.7 Hz, 1H).

### Intermediate 2

### (3S)-6′-Chloro-N,N-bis[(4-methoxyphenyl)methyl]spiro[4,5-dihydro-2H-1,5-benzoxazepine-3,1′-tetralin]-7-sulfonamide

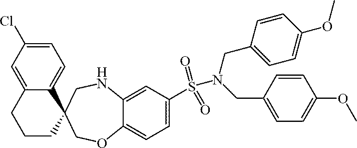 [SMILES: COc1ccc(CN(Cc2ccc(OC)cc2)S(=O)(=O)c2ccc3c(c2)NC[C@@]2(CCCc4cc(Cl)ccc42)CO3)cc1]

### Step 1: 4-fluoro-N,N-bis[(4-methoxyphenyl)methyl]-3-nitro-benzenesulfonamide

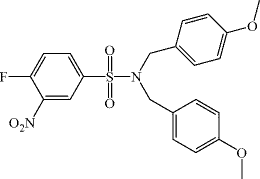 [SMILES: COc1ccc(CN(Cc2ccc(OC)cc2)S(=O)(=O)c2ccc(F)c([N+](=O)[O-])c2)cc1]

To a cooled (-35° C.) solution of 4-Fluoro-3-nitrobenzenesulfonyl chloride (4.89 g, 20.42 mmol) in THF (50 mL) was added Triethylamine (3.13 mL, 22.46 mmol), followed by addition of Bis-(4-methoxybenzyl)amine (4.97 mL, 20.7 mmol) in THF (50 mL) solution over 30 min. while the temperature was kept at -35° C. After completion of the addition, the temperature was allowed slowly to warm to 0° C. over 1 h., and the mixture was stirred at 0° C. for an additional hour. The mixture was neutralized with 1 N HCl to pH about 4-5 and diluted with EtOAc (100 mL). The organic layer was separated, washed with 1 N HCl (10 mL), 7.5% NaHCO3 aqueous solution (20 mL), and brine, dried over Na₂SO₄, filtered, and concentrated under reduced pressure. The residue was treated with DCM (30 mL), and hexane was added to the suspension until it became cloudy. The resulting suspension was sonicated for 2 min. and left at r.t. for 1 h. The mixture was filtered. and washed with hexane to afford the desired title product (6.85 g) without further purification. The mother liquid was concentrated under reduced pressure. The residue was treated with DCM (5 mL) and hexane was added as the procedures mentioned above to afford the additional 0.51 g of the title product. Total product 4-fluoro-N,N-bis[(4-methoxyphenyl)methyl]-3-nitro-benzenesulfonamide obtained is 7.36 g (78%). ¹H NMR (400 MHz, DMSO-d₆): δ 8.18-8.23 (m, 2 H), 7.75-7.79 (q, 1 H), 7.08 (d, 4 H), 6.81 (d, 4 H), 4.31 (s, 4 H), 3.71 (s, 6 H). ¹⁹F NMR (376 MHz, DMSO-d6): δ -112.54 (s, 1 F). LCMS calculated for C₂₂H₂₂FN₂O₆S (M+H)⁺: m/z = 461.11; found: 461.1.

### Step 2: [(1S)-6-chloro-1-(hydroxymethyl)tetralin-1-yl]methyl Benzoate and [(1R)-6-chloro-1-(hydroxymethyl)tetralin-1-yl]methyl Benzoate

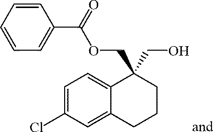

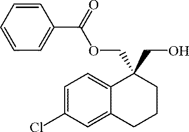 [SMILES: O=C(OC[C@]1(CO)CCCc2cc(Cl)ccc21)c1ccccc1]

Racemic product 6-chloro-1-(hydroxymethyl)tetralin-1-yl]methyl benzoate (intermediate 1, Step 4) was separated by Waters-SFC80 instrument under the separation conditions: Column: AD-H (2.5*25 cm, 10 um); Mobile phase A: Supercritical CO₂, Mobile phase B: EtOH, A:B = 80/20 at 60 mL/min; Circle Time: 15 min; Sample preparation: Ethanol; Injection Volume: 0.8 mL; Detector Wavelength: 214 nm; Column temperature: 25° C.; Back pressure: 100 bar. The separated products were determined by chiral HPLC. Chiral HPLC conditions: Chiral Column: AD-H, 5 um,4.6 mm × 250 mm (Daicel); Mobile phase: Supercritical CO₂/EtOH/DEA 70/30/0.06; Flow rate: 2.0 mL/min and Run time: 12 min. to afford [(1S)-6-chloro-1-(hydroxymethyl)tetralin-1-yl]methyl benzoate (P1, Retention time = 4.952 min.) and [(1R)-6-chloro-1-(hydroxymethyl)tetralin-1-yl]methyl benzoate (P2, Retention time = 6.410 min.). ¹H NMR (400 MHz, CDCl₃): δ 8.00-8.02 (m, 2 H), 7.57-7.61 (m, 1 H), 7.44-7.48 (m, 3 H), 7.14-7.16(m, 2 H), 4.48 (s, 2 H), 3.74-3.82 (m, 2 H), 2.78-2.81 (m, 2 H), 1.83-1.95 (m, 4 H).

### Step 3: [(1R)-6-chloro-1-formyl-tetralin-1-yl]methyl Benzoate

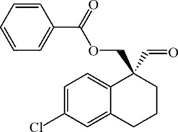 [SMILES: O=C[C@]1(COC(=O)c2ccccc2)CCCc2cc(Cl)ccc21]

This compound was prepared using procedures analogous to those described for Intermediate 1 using [(1S)-6-chloro-1-(hydroxymethyl)tetralin-1-yl]methyl benzoate (Step 2, P1) to replace the racemic [6-chloro-1-(hydroxymethyl)tetralin-1-yl]methyl benzoate in Step 5. ¹H NMR (400 MHz, CDCl₃): δ 9.61 (s, 1 H), 7.94-7.96 (m, 2 H), 7.55-7.58 (m, 1 H), 7.41-7.45 (m, 2 H), 7.15-7.20 (m, 3 H), 4.73-4.76 (d, 1H), 4.53-4.56 (d, 1H), 2.82-2.85 (m, 2 H), 2.20-2.26 (m, 1 H), 2.01-2.07 (m, 1 H), 1.90-1.96 (m, 2 H).

### Step 4: [(1R)-6-chloro-1-(dimethoxymethyl)tetralin-1-yl]methanol

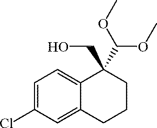 [SMILES: COC(OC)[C@]1(CO)CCCc2cc(Cl)ccc21]

Method A: This compound was prepared using procedures analogous to those described for Intermediate 1 using [(1R)-6-chloro-1-formyl-tetralin-1-yl]methyl benzoate to replace the racemic (6-chloro-1-formyl-tetralin-1-yl)methyl benzoate in Step 6. ¹H NMR (400 MHz, CDCl₃+D₂O): δ 7.34-7.36 (m, 1 H), 7.10-7.12 (m, 2 H), 4.49 (s, 1 H), 3.89-3.91 (d, 1 H), 3.50-3.53 (m, 1 H), 3.46 (s, 3 H), 3.33 (s, 3 H), 2.68-2.76 (m, 2 H), 1.99-2.06 (m, 1 H), 1.89-1.96(m, 1 H), 1.70-1.86 (m, 2 H).

Method B: The racemic (6-chloro-1-formyl-tetralin-1-yl)methyl benzoate (Intermediate 1 Step 6) was separated by chiral column on Berger MG2 Preparative SFC instrument under the separation conditions: Column: ChiralPak IC (2 × 25 cm); Mobile phase A: i-PrOH, Mobile phase B: Supercritical CO₂, A:B = ⅓ at 60 mL/min; Circle Time (Run Time): 5 min injection intervals; Sample preparation: 20 mg/mL iPrOH/DCM; Injection Volume: 0.5 mL; Detector Wavelength: 220 nm; Column temperature: 30° C.; Back pressure:100 bar. The separated products were determined by chiral HPLC on Berger Analytical SFC. Chiral HPLC conditions: Chiral Column: ChiralPak IC, 5 um, 4.6 mm x 250 mm (Daicel); Mobile phase: i-PrOH/Supercritical CO₂/EtOH ⅓; Flow rate: 3.0 mL/min and Run time: 7 min.; Detector Wavelength (UV length): 220 nm, 254 nm, and 280 nm; Column temperature: 30° C.; Back pressure: 120 bar. to afford

### [(1S)chloro-1-(dimethoxymethyl)tetralin-1-yl]methanol (P1, Retention Time = 1.96 Min.) and [(1R)-6-chloro-1-(dimethoxymethyl)tetralin-1-yl]methanol (P2, Retention time = 2.69 min.) Step 5: N,N-bis[(4-methoxyphenyl)methyl]-3-nitro-4-[[(1R)-6-chloro-1-(dimethoxymethyl)-tetralin-1-yl]methoxy]benzenesulfonamide

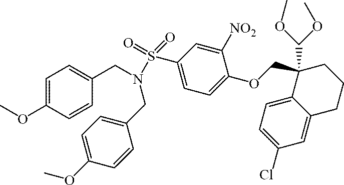 [SMILES: COc1ccc(CN(Cc2ccc(OC)cc2)S(=O)(=O)c2ccc(OC[C@@]3(C(OC)OC)CCCc4cc(Cl)ccc43)c([N+](=O)[O-])c2)cc1]

To a solution of [(1R)-6-chloro-1-(dimethoxymethyl)tetralin-1-yl]methanol (2.96 g, 10.93 mmol, P2) in THF (50 mL) was drop-wised add LiHMDS (11.5 mL, 11.4 mmol) under N₂ atmosphere at -40° C., the solution was stirred at -40° C. for 5 mins, then dropwise added 4-fluoro-N,N-bis[(4-methoxyphenyl)methyl]-3-nitro-benzenesulfonamide (7.55 g, 16.4 mmol) (Step 1) in THF (30 mL). The solution was stirred for 5 min. under -40° C., then the mixture was stirred at r.t. for 1 h. The reaction was cooled with ice-water bath, and quenched with sat. NH₄Cl aqueous solution (100 mL). The mixture was extracted with EtOAc (100 mL × 3). The combined organic layers were washed with sat. NH₄Cl solution and brine, dried over Na₂SO₄, filtered and concentrated in vacuum. The residue was purified by flash chromatography on a silica gel column eluting with ethyl acetate (EA) and petroleum ether (PE) to give N,N-bis[(4-methoxyphenyl)methyl]-3-nitro-4-[[(1R)-6-chloro-1-(dimethoxymethyl)tetralin-1-yl]methoxy]benzenesulfonamide (6.41 g, 82% yield). ¹H NMR (400 MHz, DMSO-d₆): δ 8.06-8.07 (m, 1 H), 7.97-8.00 (m, 1 H), 7.60-7.62 (m, 1 H), 7.49-7.51(m, 1 H), 7.14-7.17 (m, 2 H), 6.99-7.07 (m, 4 H), 6.77-6.79 (m, 4H), 4.62 (s, 1 H), 4.27-4.36 (m, 2 H), 4.24 (s, 4 H), 3.70 (s, 6 H), 3.39 (s, 3 H), 3.30 (s, 3 H), 2.68-2.71 (m, 2H), 1.98-2.00 (m, 1 H), 1.81-1.85 (m, 2 H), 1.71-1.73 (m, 1 H).

### Step 6: N,N-bis[(4-methoxyphenyl) methyl]-3-nitro-4-[[(1R)-6-chloro-1-formyl-tetralin-1-yl]methoxy]benzenesulfonamide

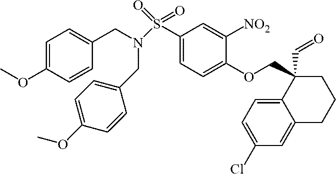 [SMILES: COc1ccc(CN(Cc2ccc(OC)cc2)S(=O)(=O)c2ccc(OC[C@@]3(C=O)CCCc4cc(Cl)ccc43)c([N+](=O)[O-])c2)cc1]

To a solution of N,N-bis[(4-methoxyphenyl)methyl]-3-nitro-4-[[(1R)-6-chloro-1-(dimethoxymethyl)tetralin-1-yl]methoxy]benzenesulfonamide (6.11 g, 8.59 mmol) in THF (80 mL) and water (20 mL) was added p-TsOH·H₂O (3.27 g, 17.18 mmol), the mixture was stirred at 70° C. for 16 h. The mixture was cooled to 0° C., and sat. NaHCO₃ aqueous (100 mL) was added. The mixture was extracted with EA (100 mL × 3). The combined organic layers were dried over Na₂SO₄, filtered and concentrated under reduced pressure. The residue was purified by flash chromatography on a silica gel column eluting with EA and to give N,N-bis[(4-methoxyphenyl) methyl]-3-nitro-4-[[(1R)-6-chloro-1-formyl-tetralin-1-yl]methoxy]benzenesulfonamide (6.11 g, 85% purity, 91% yield).

### Step 7: (S)-6′-chloro-N,N-bis(4-methoxybenzyl)-3′, 4′-dihydro-2H, 2′H-spiro[benzo[b][1, 4]oxazepine-3,1′-naphthalene]-7-sulfonamide

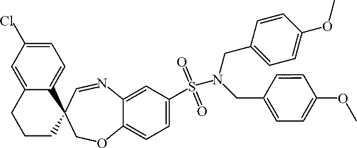 [SMILES: COc1ccc(CN(Cc2ccc(OC)cc2)S(=O)(=O)c2ccc3c(c2)N=C[C@@]2(CCCc4cc(Cl)ccc42)CO3)cc1]

To a solution of N,N-bis[(4-methoxyphenyl)methyl]-3-nitro-4-[[(1R)-6-chloro-1-formyl-tetralin-1-yl]methoxy]benzenesulfonamide (6.11 g, 7.81 mmol) in ethanol (40 mL) and water (20 mL) was added iron powder (2.18 g, 39 mmol) and NH₄Cl (827 mg, 15.6 mmol), the mixture was stirred at 100° C. for 3 h. LCMS showed the reaction completed. The mixture was filtered. The filtrate was added H₂O (20 mL), extracted with EA (30 mL × 3). The combined organic layers were dried over Na₂SO₄, filtered and concentrated under reduced pressure to give (S)-6′-chloro-N,N-bis(4-methoxybenzyl)-3′,4′-dihydro-2H,2′H-spiro[benzo[b][1,4]oxazepine-3,1′-naphthalene]-7-sulfonamide (6.11 g, 70% purity, 86% yield) which was directly used in next step reaction without further purification. LCMS calculated for C₃₄H₃₄ClN₂O₅S (M+H)⁺: m/z = 617.18; found: 617.3.

### Step 8: (3S)-6′-chloro-N,N-bis[(4-methoxyphenyl)methyl]spiro[4,5-dihydro-2H-1,5-benzoxazepine-3,1′-tetralin]-7-sulfonamide

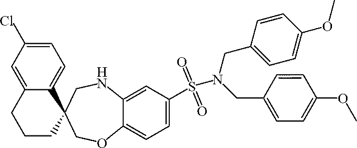 [SMILES: COc1ccc(CN(Cc2ccc(OC)cc2)S(=O)(=O)c2ccc3c(c2)NC[C@@]2(CCCc4cc(Cl)ccc42)CO3)cc1]

To a solution of (S)-6′-chloro-N,N-bis(4-methoxybenzyl)-3′,4′-dihydro-2H,2′H-spiro[benzo[b][1,4]oxazepine-3,1′-naphthalene]-7-sulfonamide (6.11 g, 6.73 mmol) (crude product from Step 7, 70% purity) in DCM (80 mL) was portion-wise added NaBH(OAc)₃ (7.14 g, 33.67 mmol). The mixture was stirred at 25° C. for 16 h. LCMS showed the reaction worked well. The reaction was added sat. NaHCO₃ aqueous (80 mL), extracted with DCM (100 mL × 3), dried over Na₂SO₄, filtered and concentrated under reduced pressure. The residue was purified by flash chromatography on a silica gel column eluting with EA and PE to give (3S)-6′-chloro-N,N-bis[(4-methoxyphenyl)methyl]spiro[4,5-dihydro-2H-1,5-benzoxazepine-3,1′-tetralin]-7-sulfonamide (2.30 g, 53% yield). LCMS calculated for C₃₄H₃₆ClN₂O₅S (M+H)⁺: m/z = 619.2; found: 619.3. ¹H NMR (400 MHz, DMSO-d₆): δ 7.81-7.83 (m, 1 H), 7.24-7.28 (m, 2 H), 7.17-7.18 (m, 1 H), 6.95-7.06 (m, 6 H), 6.78-6.80 (m, 4 H), 6.20 (s, 1 H), 4.15 (m, 4 H), 4.08-4.14 (m, 2 H), 3.68 (s, 6 H), 3.30-3.36 (m, 1 H), 3.23-3.27 (m, 1 H), 2.71-2.75 (m, 2 H), 1.76-1.86 (m, 3 H), 1.56-1.61 (m, 1 H).

### Intermediate 3

### (3S)-6′-Chloro-5-[[(1R,2R)-2-[(1S)-1-hydroxyallyl]cyclobutyl]methyl]spiro[2,4-dihydro-1,5-benzoxazepine-3,1′-tetralin]-7-sulfonamide

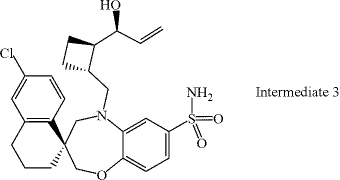 [SMILES: C=C[C@H](O)[C@@H]1CC[C@H]1CN1C[C@@]2(CCCc3cc(Cl)ccc32)COc2ccc(S(N)(=O)=O)cc21]

### Step 1: [(1R, 2R)-2-[[(3S)-7-[bis[(4-methoxyphenyl)methyl]sulfamoyl]-6′-chloro-spiro[2, 4-dihydro-1,5-benzoxazepine-3, 1′-tetralin]-5-yl]methyl]cyclobutyl]methyl Acetate

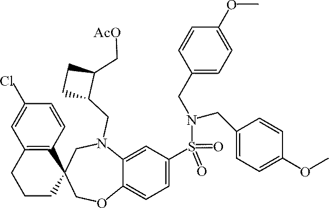 [SMILES: COc1ccc(CN(Cc2ccc(OC)cc2)S(=O)(=O)c2ccc3c(c2)N(C[C@@H]2CC[C@H]2COC(C)=O)C[C@@]2(CCCc4cc(Cl)ccc42)CO3)cc1]

2,2,2-trifluoroacetic acid (7.0 mL, 92 mmol) was dropwise added to a stirred solution of sodium borohydride (3.48 g, 92.0 mmol) in DCM (200 mL) at 0° C. The resulting mixture was stirred at 0° C. for 10 min. A solution of (3S)-6′-chloro-N,N-bis[(4-methoxyphenyl)methyl]spiro[4,5-dihydro-2H-1,5-benzoxazepine-3,1′-tetralin]-7-sulfonamide (28.5 g, 46.03 mmol) and [(1R,2R)-2-formylcyclobutyl]methyl acetate (8.63 g, 55.24 mmol) in 200 mL DCM was then dropwise added at 0° C. The resulting mixture was stirred at room temperature for overnight. The reaction was monitored by LC-MS. Another 2 equivalents of sodium borohydride (3.48 g, 92.06 mmol) and 2,2,2-trifluoroacetic acid (7.04 mL, 92.06 mmol) were added to the mixture, followed by the stirring for 3 h. The reaction was quenched by addition of methanol (30 mL), and followed by addition of saturated NaHCO₃ solution (300 mL) slowly. The resulting mixture was extracted with DCM (300 mL × 3). The combined organic layers were dried over Na₂SO₄, filtered and concentrated under reduced pressure. The residue was purified by flash chromatography on a silica gel column using EtOAc/Heptanes (5 - 40%) to afford the desired product [(1R,2R)-2-[[(3S)-7-[bis[(4-methoxyphenyl)methyl]sulfamoyl]-6′-chloro-spiro[2,4-dihydro-1,5-benzoxazepine-3,1′-tetralin]-5-yl]methyl]cyclobutyl]methyl acetate (34.5 g, 45.4 mmol, 98% yield) as a white solid. LC-MS calc. for C₄₀H₄₃ClN₂O₆S [M+H]⁺: m/z = 759.28/760.28; Found 759.67/760.64.

### Step 2: (3S)-6′-chloro-N,N-bis[(4-methoxyphenyl)methyl]-5-[[(1R,2R)-2-(hydroxymethyl)cyclobutyl]methyl]spiro[2, 4-dihydro-1, 5-benzoxazepine-3,1′-tetralin]-7-sulfonamide

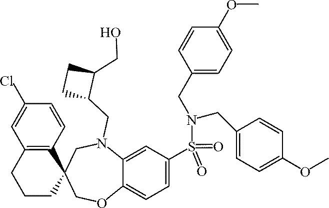 [SMILES: COc1ccc(CN(Cc2ccc(OC)cc2)S(=O)(=O)c2ccc3c(c2)N(C[C@@H]2CC[C@H]2CO)C[C@@]2(CCCc4cc(Cl)ccc42)CO3)cc1]

To a solution of [(1R,2R)-2-[[(3S)-7-[bis[(4-methoxyphenyl)methyl]sulfamoyl]-6′-chloro-spiro[2,4-dihydro-1,5-benzoxazepine-3,1′-tetralin]-5-yl]methyl]cyclobutyl]methyl acetate (54.0 g, 71.1 mmol) in THF (500 mL), methanol (500 mL) and water (500 mL) was added lithium hydroxide monohydrate (14.9 g, 355 mmol). The mixture was stirred at r.t. overnight. The solvent was removed, and the aqueous layer was extracted with DCM (100 mL x 3). The combined organic layers were dried over Na₂SO₄, filtered and concentrated under reduced pressure to afford (3S)-6′-chloro-N,N-bis[(4-methoxyphenyl)methyl]-5-[[(1R,2R)-2-(hydroxymethyl)cyclobutyl]methyl]spiro[2,4-dihydro-1,5-benzoxazepine-3,1′-tetralin]-7-sulfonamide (52 g, 101% yield) as white solid which was directly used for the next step without further purification. LC-MS calc. for C₄₀H₄₅ClN₂O₆S [M+H]⁺: m/z = 717.27/718.27; Found 717.6/718.6.

### Step 3: (3S)-6′-chloro-N,N-bis[(4-methoxyphenyl)methyl]-5-[[(1R,2R)-2-formylcyclobutyl]methyl]spiro[2, 4-dihydro-1, 5-benzoxazepine-3,1 ‘-tetralin]-7-sulfonamide

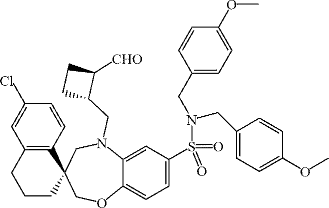 [SMILES: COc1ccc(CN(Cc2ccc(OC)cc2)S(=O)(=O)c2ccc3c(c2)N(C[C@@H]2CC[C@H]2C=O)C[C@@]2(CCCc4cc(Cl)ccc42)CO3)cc1]

DMSO (20.5 mL, 289 mmol) was slowly added to a cooled (-78° C.) solution of oxalyl chloride (12.4 mL, 144.9 mmol) in DCM (1000 mL). Gas was produced during this addition. The mixture was stirred at -78° C. for 30 min. Then a solution of (3S)-6′-chloro-N,N-bis[(4-methoxyphenyl)methyl]-5-[[(1R,2R)-2-(hydroxymethyl)cyclobutyl]methyl]spiro[2,4-dihydro-1,5-benzoxazepine-3,1′-tetralin]-7-sulfonamide (52.0 g, 72.4 mmol) in DCM (50 mL) was added over 5 min. The resulting mixture was stirred at -78° C. for 40 min. Then triethylamine (101 mL, 724 mmol) was added. The solution was stirred at -78° C. for additional 10 min, and allowed to warme slowly to 0° C. After the starting material was consumed, water (150 mL) was added. The organic layer were separated. The aqueous layer was extracted with DCM (300 mL × 3). The combined organic layers were dried over sodium sulfate and concentrated. The residue was purified by flash chromatography on a silica gel column with EtOAc/Heptanes (5 - 50%) to afford (3S)-6′-chloro-N,N-bis[(4-methoxyphenyl)methyl]-5-[[(1R,2R)-2-formylcyclobutyl]methyl]spiro[2,4-dihydro-1,5-benzoxazepine-3,1′-tetralin]-7-sulfonamide (43 g, 83% yield) as a white solid. LC-MS calc. for C₄₀H₄₃ClN₂O₆S [M+H]⁺: m/z = 715.25/716.26; Found 715.7/716.7.

### Step 4: (3S)-6′-chloro-N,N-bis[(4-methoxyphenyl)methyl]-5-[[(1R,2R)-2-[(1S)-1-hydroxyallyl]cyclobutyl]methyl]spiro[2, 4-dihydro-1, 5-benzoxazepine-3,1′-tetralin]-7-sulfonamide and (3S)-6′-chloro-N,N-bis[(4-methoxyphenyl)methyl]-5-[[(1R,2R)-2-[(1R)-1-hydroxyallyl]cyclobutyl]methyl]spiro[2, 4-dihydro-1, 5-benzoxazepine-3,1′-tetralin]-7-sulfonamide

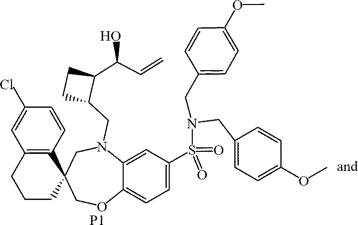

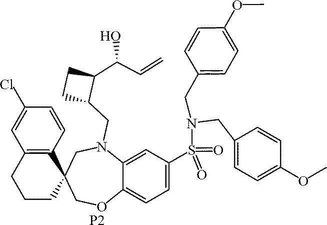

Vinylmagnesium bromide (1.0 M solution in THF, 300 mL, 300 mmol) was diluted with THF (200 mL) in a 3 necked round bottom flack under nitrogen. (3S)-6′-chloro-N,N-bis[(4-methoxyphenyl)methyl]-5-[[(1R,2R)-2-formylcyclobutyl]methyl]spiro[2,4-dihydro-1,5-benzoxazepine-3,1′-tetralin]-7-sulfonamide (43.0 g, 60.1 mmol) dissolved in THF (400 mL) was introduced dropwise through a dropping funnel over 2 hours at room temperature. The reaction was monitored by LC-MS. After the starting material was consumed, the reaction was then quenched by addition of sat. aqueous solution NH₄Cl (300 mL) at 0° C. The organic layer was then separated, and the aqueous layer was extracted with ethyl acetate (300 mL × 2). The combined organic layers were dried over Na₂SO₄, filtered and concentrated under reduced pressure. The residue was purified by flash chromatography on a silica gel column using EtOAc/Heptanes (5 - 40%) to afford two products: P1 (the earlier eluted product: 24.3 g, 40%) and P2 (the latter eluted product: 20 g, 33%).

P1 was assigned as (3S)-6′-chloro-N,N-bis[(4-methoxyphenyl)methyl]-5-[[(1R,2R)-2-[(1S)-1-hydroxyallyl]cyclobutyl]methyl]spiro[2,4-dihydro-1,5-benzoxazepine-3,1′-tetralin]-7-sulfonamide (Rt = 4.43 min from LC-MS). LC-MS calc. for C₄₂H₄₈ClN₂O₆S [M+H]⁺: m/z = 743.28/744.29; Found 743.76/744.78. ¹H NMR (300 MHz, CDCl₃) δ 7.76 (t, J = 7.2 Hz, 1H), 7.53 (d, J = 1.9 Hz, 1H), 7.24 - 7.14 (m, 2H), 7.12 (d, J = 2.0 Hz, 1H), 7.03 - 6.97 (m, 5H), 6.79 (t, J= 5.7 Hz, 4H), 5.84 - 5.69 (m, 1H), 5.16 (d, J = 17.2 Hz, 1H), 5.05 (d, J = 10.4 Hz, 1H), 4.26 (t, J= 5.6 Hz, 4H), 4.13 (s, 2H), 3.97 (d, J = 4.4 Hz, 1H), 3.80 (d, J= 1.8 Hz, 6H), 3.74 (d, J= 6.2 Hz, 1H), 3.26 (d, J = 14.2 Hz, 1H), 3.09 (dd, J = 15.0, 9.3 Hz, 1H), 2.93 (d, J = 4.2 Hz, 1H), 2.83 - 2.75 (m, 2H), 2.48 - 2.35 (m, 1H), 2.10 - 1.92 (m, 4H), 1.82 (m, 3H), 1.50 (m, 2H).

And P2 was assigned as (3S)-6′-chloro-N,N-bis[(4-methoxyphenyl)methyl]-5-[[(1R,2R)-2-[(1R)-1-hydroxyallyl]cyclobutyl]methyl]spiro[2,4-dihydro-1,5-benzoxazepine-3,1′-tetralin]-7-sulfonamide (Rt = 4.13 min from LC-MS). LC-MS calc. for C₄₂H₄₈ClN₂O₆S [M+H]⁺: m/z = 743.28/745.29; Found 743.8/745.8. ¹H NMR (300 MHz, CDCl₃) δ 7.75 - 7.68 (m, 1H), 7.24 - 7.14 (m, 3H), 7.12 (d, J = 2.0 Hz, 1H), 7.01 (t, J = 8.3 Hz, 5H), 6.79 (d, J = 8.7 Hz, 4H), 5.85 (ddd, J= 17.0, 10.4, 6.4 Hz, 1H), 5.29 (dd, J = 17.2, 1.2 Hz, 1H), 5.17 - 5.08 (m, 1H), 4.26 (d, J= 8.4 Hz, 4H), 4.14 (d, J = 8.0 Hz, 3H), 3.81 (s, 6H), 3.69 (d, J = 14.3 Hz, 1H), 3.59 (d, J= 12.9 Hz, 1H), 3.31 (d, J = 14.3 Hz, 1H), 3.15 (dd, J = 14.9, 9.0 Hz, 1H), 2.84 - 2.76 (m, 2H), 2.67 - 2.56 (m, 1H), 2.23 - 2.09 (m, 2H), 2.03 (m, 2H), 1.86 - 1.73 (m, 3H), 1.59 - 1.46 (m, 2H).

### Step 5: (3S)-6′-chloro-5-[[(1R,2R)-2-[(1S)-1-hydroxyallyl]cyclobutyl]methyl]spiro[2,4-dihydro-1,5-benzoxazepine-3,1′-tetralin]-7-sulfonamide (Intermediate 3)

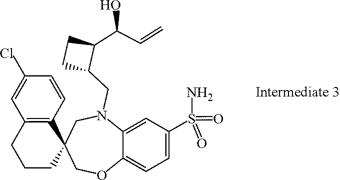 [SMILES: C=C[C@H](O)[C@@H]1CC[C@H]1CN1C[C@@]2(CCCc3cc(Cl)ccc32)COc2ccc(S(N)(=O)=O)cc21]

To a solution of (3S)-6′-chloro-N,N-bis[(4-methoxyphenyl)methyl]-5-[[(1R,2R)-2-[(1S)-1-hydroxyallyl]cyclobutyl]methyl]spiro[2,4-dihydro-1,5-benzoxazepine-3,1′-tetralin]-7-sulfonamide (24.3 g, 32.6 mmol, P1, Step 4) and anisole (23.7 mL, 218 mmol) in DCM (240 mL) was added 2,2,2-trifluoroacetic acid (243 mL). The mixture was stirred overnight. The reaction was monitored by LC-MS. Solvents were removed under reduced pressure. The residue was diluted with DCM (200 mL). The mixture was washed with saturated aqueous NaHCO₃ solution (200 mL × 3) and brine, dried over Na₂SO₄, filtered and concentrated under reduced pressure. The residue was purified by flash chromatography on a silica gel column with EA/heptane (5%-70%) to afford (3S)-6′-chloro-5-[[(1R,2R)-2-[(1S)-1-hydroxyallyl]cyclobutyl]methyl]spiro[2,4-dihydro-1,5-benzoxazepine-3,1′-tetralin]-7-sulfonamide (15.7 g, 31.2 mmol, 95% yield) as a pale white solid. LC-MS calc. for C₂₆H₃₂ClN₂O₄S [M+H]⁺: m/z = 503.17/505.17; Found 503.5/505.5; ¹H NMR (300 MHz, CDCl₃) δ 7.74 (d, J = 8.5 Hz, 1H), 7.55 (d, J = 1.8 Hz, 1H), 7.21 (dd, J = 11.4, 4.2 Hz, 2H), 7.12 - 7.08 (m, 2H), 6.97 - 6.94 (m, 1H), 6.85 (d, J = 8.6 Hz, 1H), 5.90 - 5.76 (m, 1H), 5.25 (d, J = 17.2 Hz, 1H), 5.16 - 5.08 (m, 1H), 4.11 (s, 2H), 3.88 (d, J = 5.1 Hz, 1H), 3.81 (s, 2H), 3.27 (d, J = 14.3 Hz, 1H), 3.14 (m, 1H), 2.84 - 2.75 (m, 2H), 2.51 (dd, J= 16.9, 8.5 Hz, 1H), 2.08 (m, 3H), 1.90 (dd, J= 15.8, 5.6 Hz, 2H), 1.63 (m, 3H), 1.45 (t, J= 12.1 Hz, 1H).

### 2-Allyloxy-2-methyl-propanoic Acid

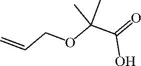

This compound can be prepared by treating ethyl 2-hydroxy-2-methyl-propanoate with NaH in THF, followed by reaction with allyl bromide. The resulting product is then reacted with sodium hydroxide to give 2-allyloxy-2-methyl-propanoic acid.

### Example 32

### (3R,6R,7S,8E,22S)-6′-Chloro-7-hydroxy-12,12-dimethyl-15,15-dioxo-spiro[11,20-dioxa-15-thia-1,14-diazatetracyclo [14.7.2.03,6.019,24] pentacosa-8,16,18,24-tetraene-22,1′-tetralin]-13-one

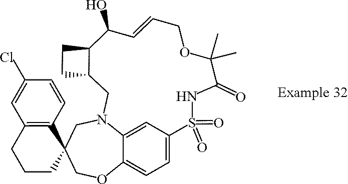 [SMILES: CC1(C)OC/C=C/[C@H](O)[C@@H]2CC[C@H]2CN2C[C@@]3(CCCc4cc(Cl)ccc43)COc3ccc(cc32)S(=O)(=O)NC1=O]

### Step 1: [(1S)-1-[(1R,2R)-2-[[(3S)-7-[(2-allyloxy-2-methyl-propanoyl)sulfamoyl]-6′-chloro-spiro[2, 4-dihydro-1, 5-benzoxazepine-3,1 ‘-tetralin]-5-yl]methyl]cyclobutyl]allyl] 2-allyloxy-2-methyl-propanoate

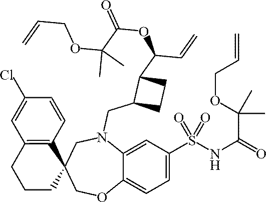 [SMILES: C=CCOC(C)(C)C(=O)NS(=O)(=O)c1ccc2c(c1)N(C[C@@H]1CC[C@H]1[C@H](C=C)OC(=O)C(C)(C)OCC=C)C[C@@]1(CCCc3cc(Cl)ccc31)CO2]

A solution of (3S)-6′-chloro-5-[[(1R,2R)-2-[(1S)-1-hydroxyallyl]cyclobutyl]methyl]spiro[2,4-dihydro-1,5-benzoxazepine-3,1′-tetralin]-7-sulfonamide (200.0 mg, 0.40 mmol, Intermediate 3), 2-allyloxy-2-methyl-propanoic acid (171.96 mg, 1.19 mmol), EDCI (0.47 mL, 2.39 mmol), and DMAP (291.43 mg, 2.39 mmol) in DCM (4 mL) was stirred at r.t. for 16 h. LC-MS indicated the completion of reaction. The reaction was diluted with DCM and washed with 0.5 N HCl. The organic phase was dried over Na₂SO₄ and concentrated under reduced pressure. The residue was purified by flash chromatography on a silica gel column (12 g) with EtOAc/Heptanes (10% to 20%) to afford [(1S)-1-[(1R,2R)-2-[[(3S)-7-[(2-allyloxy-2-methyl-propanoyl)sulfamoyl]-6′-chloro-spiro[2,4-dihydro-1,5-benzoxazepine-3,1′-tetralin]-5-yl]methyl]cyclobutyl]allyl] 2-allyloxy-2-methyl-propanoate (300 mg, 99.9% yield). LC-MS: calc. for C₄₀H₅₂ClN₂O₈S [M+H]⁺: m/z = 755.31/757.31; Found: 755.1/757.4.

### Step 2: 2-allyloxy-2-methyl-N-[(3S)-6′-chloro-5-[[(1R,2R)-2-[(1S)-1-hydroxyallyl]cyclobutyl]methyl]spiro[2, 4-dihydro-1, 5-benzoxazepine-3, 1′-tetralin]-7-yl]sulfonyl-propanamide

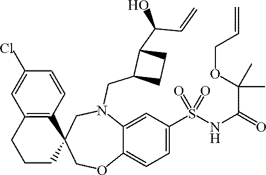 [SMILES: C=CCOC(C)(C)C(=O)NS(=O)(=O)c1ccc2c(c1)N(C[C@@H]1CC[C@H]1[C@@H](O)C=C)C[C@@]1(CCCc3cc(Cl)ccc31)CO2]

A solution of [(1S)-1-[(1R,2R)-2-[[(3S)-7-[(2-allyloxy-2-methylpropanoyl)sulfamoyl]-6′-chloro-spiro[2,4-dihydro-1,5-benzoxazepine-3,1′-tetralin]-5-yl]methyl]cyclobutyl]allyl] 2-allyloxy-2-methyl-propanoate (300 mg, 0.40 mmol) and lithium hydroxide monohydrate (83.3 mg, 1.99 mmol) in THF/MeOH/H₂O (0.3 mL each) was heated at 45° C. for 4 h. LC-MS indicated the completion of reaction. The reaction was adjusted with 1 N HCl to pH 3-4 and extracted with DCM. The combined organic layers were washed with saturated aqueous NaHCO₃ solution and brine, dried over Na₂SO₄, filtered and concentrated under reduced pressure to afford 2-allyloxy-2-methyl-N-[(3S)-6′-chloro-5-[[(1R,2R)-2-[(1S)-1-hydroxyallyl]cyclobutyl]methyl]spiro[2,4-dihydro-1,5-benzoxazepine-3,1′-tetralin]-7-yl]sulfonyl-propanamide (175 mg, 70% yield), which was used without further purifications. LCMS: calc. for C₃₃H₄₂ClN₂O₆S [M+H]⁺: m/z =629.24/631.24; Found: 628.9/631.2.

### Step 3: (3R,6R, 7S, 8E,22S)-6′-chloro-7-hydroxy-12, 1 2-dimethyl-15, 1 5-dioxo-spiro[11, 20-dioxa-15-thia-1, 14-diazatetracyclo[14.7.2.03,6.019,24]pentacosa-8, 16, 18, 24-tetraene-22, 1′-tetralin]-13-one

A solution of 2-allyloxy-N-[(3S)-6′-chloro-5-[[(1R,2R)-2-[(1S)-1-hydroxyallyl]cyclobutyl]methyl]spiro[2,4-dihydro-1,5-benzoxazepine-3,1′-tetralin]-7-yl]sulfonyl-2-methyl-propanamide (1.40 g, 2.23 mmol) in DCE (1230 mL) was bubbled with N₂ for 10 min. 1,3-Bis(2,4,6-trimethylphenyl)-4,5-dihydroimidazol-2-ylidene[2-(i-propoxy)-5-(N,N-dimethyl aminosulfonyl)phenyl]methyleneruthenium(II) dichloride (Zhan Catalyst 1B) (326 mg, 0.45 mmol) was added and the resulting greenish solution was further bubbled with N₂ for 5 min., and was heated at 40° C. under N₂ for 2 h. The reaction was concentrated under reduced pressure, and the residue purified by flash column chromatography on a silica gel column with EtOAc/Hept (10% to 70%) to afford two products: P1 (the earlier eluted product, 160 mg, 11% yield) and P2 (the latter eluted product, 647 mg, 47% yield).

P2 was assigned to (3R,6R,7S,8E,22S)-6′-chloro-7-hydroxy-12,12-dimethyl-15,15-dioxo-spiro[11,20-dioxa-15-thia-1,14-diazatetracyclo[14.7.2.03,6.019,24]pentacosa-8,16,18,24-tetraene-22,1′-tetralin]-13-one (Example 32). HPLC: major product, C18 column (4.6 × 150 mm, 100 Å); flow rate = 1 mL/ min; mobile phase: 90% MeCN/H₂O (with 0.1% HCO₂H) 10 min λ = 220 nm. tR = 3.2 min. LC-MS calc. for C₃₁H₃₈ClN₂O₆S [M+H]⁺: m/z = 601.21/603.21; Found 601.6/603.6; ¹H NMR (300 MHz, CDCl₃) δ 9.15 (s, 1H), 7.69 (d, J = 8.5 Hz, 1H), 7.53 (dd, J = 8.3, 2.1 Hz, 1H), 7.20 (dd, J= 8.6, 2.2 Hz, 1H), 7.12 (s, 1H), 7.06 (d, J = 1.8 Hz, 1H), 7.02 (d, J = 8.3 Hz, 1H), 5.84 - 5.72 (m, 2H), 4.24 (d, J= 3.3 Hz, 1H), 4.13 (t, J= 7.2 Hz, 2H), 4.00 (dd, J = 13.2, 4.5 Hz, 1H), 3.88 (d, J= 12.5 Hz, 1H), 3.72 (d, J = 14.6 Hz, 1H), 3.40 - 3.24 (m, 3H), 2.84 - 2.71 (m, 3H), 2.43 - 2.33 (m, 1H), 2.01 (d, J= 15.5 Hz, 2H), 1.94 - 1.81 (m, 4H), 1.75 -1.58 (m, 2H), 1.54 (d, J= 14.5 Hz, 1H), 1.45 (s, 3H), 1.41 (s, 3H).

And P1 to (3R,6R,7S,8Z,22S)-6′-chloro-7-hydroxy-12,12-dimethyl-15,15-dioxo-spiro[11,20-dioxa-15-thia-1,14-diazatetracyclo[14.7.2.03,6.019,24]pentacosa-8,16,18,24-tetraene-22,1′-tetralin]-13-one (Example 33). P1: minor product, C18 column (4.6 × 150 mm, 100 Å); flow rate = 1 mL/ min; mobile phase: 90% MeCN/H₂O (with 0.1% HCO₂H) 10 min λ= 220 nm. tR = 4.3 min. LC-MS calc. for C₃₁H₃₈ClN₂O₆S [M+H]⁺: m/z = 601.21/603.21; Found 601.6/603.6; ¹H NMR (300 MHz, CDCl₃) δ 9.22 (s, 1H), 7.68 (t, J = 8.3 Hz, 1H), 7.55 (dd, J = 8.4, 2.1 Hz, 1H), 7.20 (dd, J = 8.5, 2.1 Hz, 1H), 7.13 (dd, J = 9.6, 2.0 Hz, 2H), 7.03 (d, J= 8.4 Hz, 1H), 5.92 - 5.75 (m, 2H), 4.22 - 4.14 (m, 1H), 4.00 (dd, J= 13.4, 4.9 Hz, 1H), 3.89 (dd, J= 13.3, 2.9 Hz, 1H), 3.81 - 3.61 (m, 4H), 3.33 (d, J= 14.5 Hz, 1H), 3.15 (dd, J = 15.1, 9.2 Hz, 1H), 2.79 (d, J= 9.2 Hz, 2H), 2.53 (d, J= 5.2 Hz, 1H), 2.33 - 2.22 (m, 1H), 2.08 - 1.92 (m, 4H), 1.81 (dd, J= 35.4, 6.4 Hz, 2H), 1.71 - 1.57 (m, 2H), 1.45 (s, 3H), 1.42 (s, 3H).

### Formula (I)

### [(3R,6R,7S,8E,22S)-6′-Chloro-12,12-dimethyl-13,15,15-trioxo-spiro [11,20-dioxa-15-thia-1,14-diazatetracyclo[14.7.2.03,6.019,24]pentacosa-8,16,18,24-tetraene-22,1′-tetralin]-7-yl] N,N-dimethylcarbamate

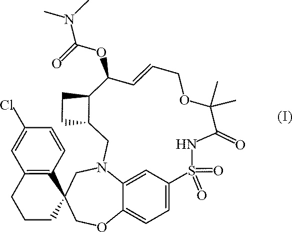 [SMILES: CN(C)C(=O)O[C@H]1/C=C/COC(C)(C)C(=O)NS(=O)(=O)c2ccc3c(c2)N(C[C@@H]2CC[C@H]21)C[C@@]1(CCCc2cc(Cl)ccc21)CO3]

To a solution of (3R,6R,7S,8E,22S)-6′-chloro-7-hydroxy-12,12-dimethyl-15,15-dioxo-spiro[11,20-dioxa-15-thia-1,14-diazatetracyclo[14.7.2.03,6.019,24]pentacosa-8,16,18,24-tetraene-22,1′-tetralin]-13-one (13.0 mg, 0.02 mmol, Example 32) in THF (0.5 mL) was added sodium hydride (4.3 mg, 0.11 mmol) at r.t.. After 10 min, N,N-dimethylcarbamoyl chloride (4.6 mg, 0.04 mmol) was added, and followed by DMAP (5.3 mg, 0.04 mmol). The mixture was stirred at r.t. for 6 h., and diluted with DCM and acidified with 0.5 N HCl to pH 5-6. The organic phase was separated, washed with water and brine, dried over Na₂SO₄, filtered and concentrated under reduced pressure. The residue was purified by prep-HPLC on C18 column (30 × 250 mm, 10 µm) with 20 to 100% ACN/H₂O to afford [(3R,6R,7S,8E,22S)-6′-chloro-12,12-dimethyl-13,15,15-trioxo-spiro[11,20-dioxa-15-thia-1,14-diazatetracyclo[14.7.2.03,6.019,24]pentacosa-8,16,18,24-tetraene-22,1′-tetralin]-7-yl] N,N-dimethylcarbamate (6 mg, 38% yield) as a white solid. LCMS: calc. for C₃₄H₄₃ClN₃O₇S [M+H]⁺: m/z = 672.25/674.25; Found: 672.45/674.37. ¹H NMR (600 MHz, CDCl₃) δ 9.08 (br s, 1H), 7.67 (d, J = 8.5 Hz, 1H), 7.49 (dd, J = 8.3, 2.2 Hz, 1H), 7.17 (dd, J = 8.5, 2.4 Hz, 1H), 7.08 (d, J = 2.2 Hz, 1H), 7.04 - 6.95 (m, 2H), 5.86 - 5.78 (m, 1H), 5.74 - 5.67 (m, 1H), 5.30 (t, J = 4.5 Hz, 1H), 4.15 (d, J = 12.2 Hz, 1H), 4.12 - 4.05 (m, 2H), 3.76 - 3.72 (m, 1H), 3.70 (d, J = 14.8 Hz, 1H), 3.43 (dd, J = 15.1, 4.7 Hz, 1H), 3.37 (d, J = 14.7 Hz, 1H), 3.21 (dd, J = 15.1, 9.3 Hz, 1H), 2.95 (d, J = 14.6 Hz, 6H), 2.83 - 2.73 (m, 3H), 2.37 (dtd, J = 15.2, 10.2, 9.7, 5.5 Hz, 1H), 2.06 - 1.90 (m, 3H), 1.88 - 1.77 (m, 3H), 1.67 - 1.60 (m, 2H), 1.56 (s, 2H), 1.43 (s, 6H).

### Prepartaion of Crystalline Forms

### Formula I - Form I - Method 1

Formula I (24.37 mg (0.036 mmol); amorphous) was added to a 4 mL vial. Methanol (1.0 mL) was added to give an almost clear solution. The mixture was stirred at 50° C. overnight to give a slurry. The slurry was cooled to room temperature and stirred for 4 h. The mixture was filtered and the cake was dried at 40-45° C. under vacuum overnight to yield 18.1 mg (74.27%) of Formula I- Form I.

XRPD: FIG. 1.

DSC: FIG. 2.

TGA: FIG. 3.

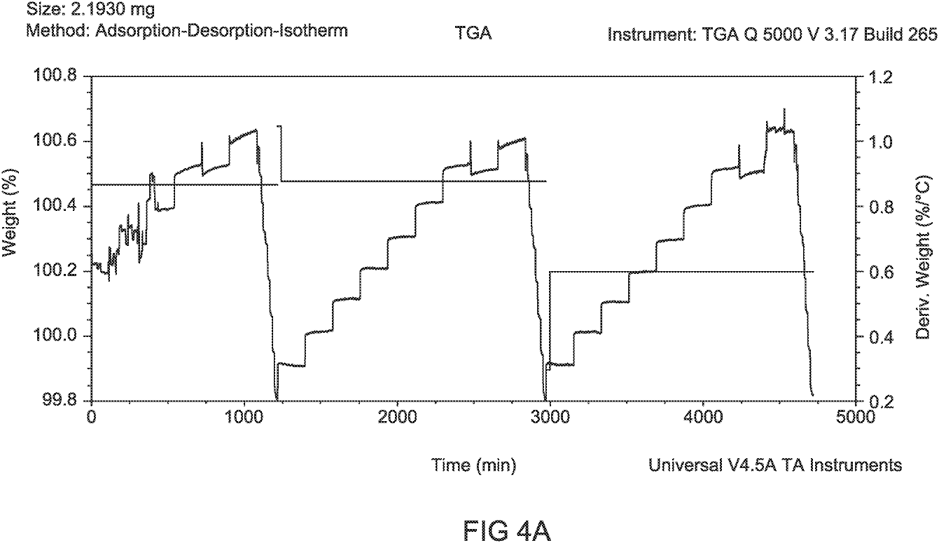

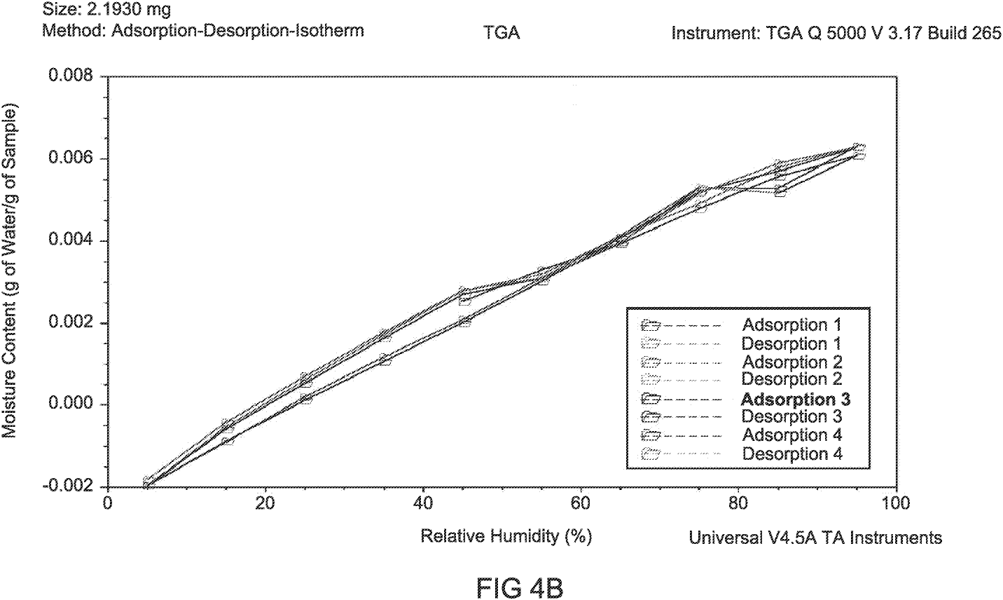

DVS: FIGS. 4A and 4B.

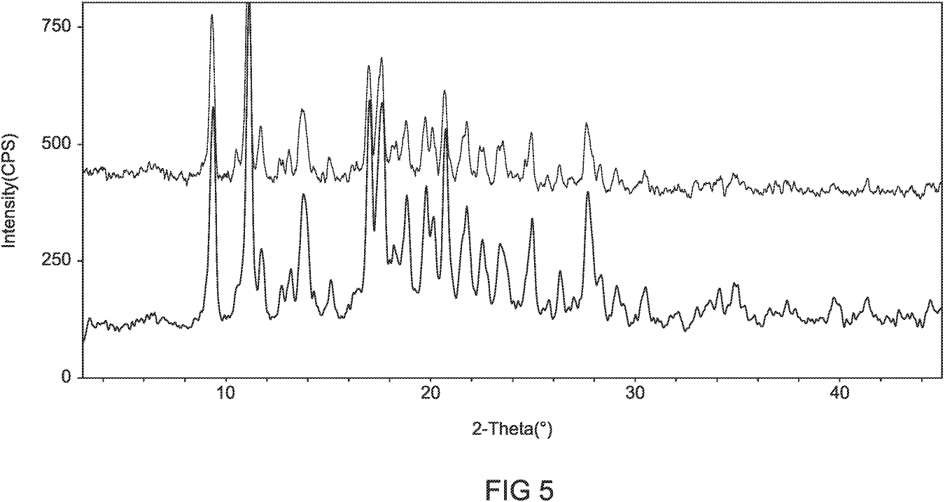

XRPD before and after DVS: FIG. 5.

### Formula I - Form I - Method 2

Formula I (23.7 mg (0.036 mmol) amorphous) was added to a 4 mL vial. Methanol (0.4 mL) and water (0.1 mL) were added to give a thin slurry. The mixture was stirred at 50° C. for 3 h to form a slurry. The mixture was cooled to room temperature and stirred for 20 min. The mixture was filtered to give Formula I-Form I.

### Formula I - Form II - Method 1

Formula I (400 mg; amorphous) was added to a 20 mL vial. Ethanol (7.0 mL) was added to give a slurry. The mixture was stirred at 70° C. for 20 minutes to give a solution. The solution was slowly cooled to give a slurry. The slurry was held over the weekend and then filtered to give Formula I- Form II.

XRPD: FIG. 6.

DSC: FIG. 7.

Drying the solid at 45-46° C. under vacuum overnight gave amorphous Formula I.

### Formula I Choline Salt (Formula IA)

Formula I (168.0 mg (0.25 mmol, 1.0 eq.) amorphous) was added to a 25 mL vial. Ethyl acetate (4.0 mL) was added to give a clear solution. 275 µL of 1.0 M choline hydroxide in IPA (0.275 mmol, 1.1 eq.) was added. The mixture was stirred for 5 minutes to give a clear solution. The mixture was continuously stirred overnight to give a slurry. The mixture was filtered and the cake was dried at room temperature under vacuum overnight to yield 150.2 mg (77.4%) of the choline salt of Formula I.

XRPD: FIG. 8.

DSC: FIG. 9.

TGA: FIG. 10

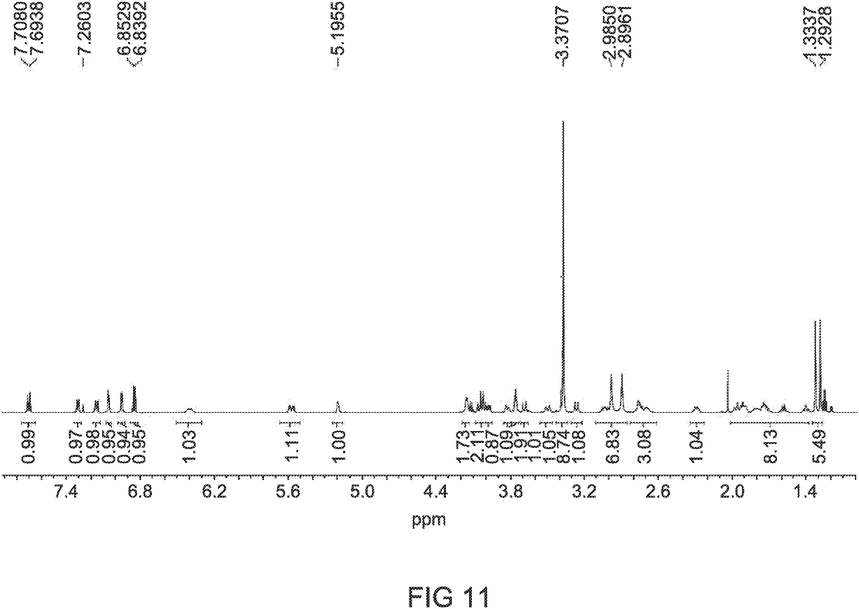

NMR spectrum (600 MHz in CDCl₃): FIG. 11.

### Formula I Benzathine Salt (Formula IB)

Formula I (168.0 mg (0.25 mmol, 1.0 eq.) amorphous) was added to a 25 mL vial. Ethyl acetate (4.0 mL) was added to give a clear solution. 275 µL of 1.0 M benzathine in IPA (0.275 mmol, 1.1 eq.) was added. The mixture was stirred for 5 minutes to give a clear solution. The mixture was continuously stirred overnight to give a slurry. The mixture was filtered and the cake was dried at room temperature under vacuum overnight to yield 100.2 mg (44.0%) of the benzathine salt of Formula I.

XRPD: FIG. 12.

DSC: FIG. 13.

TGA: FIG. 14

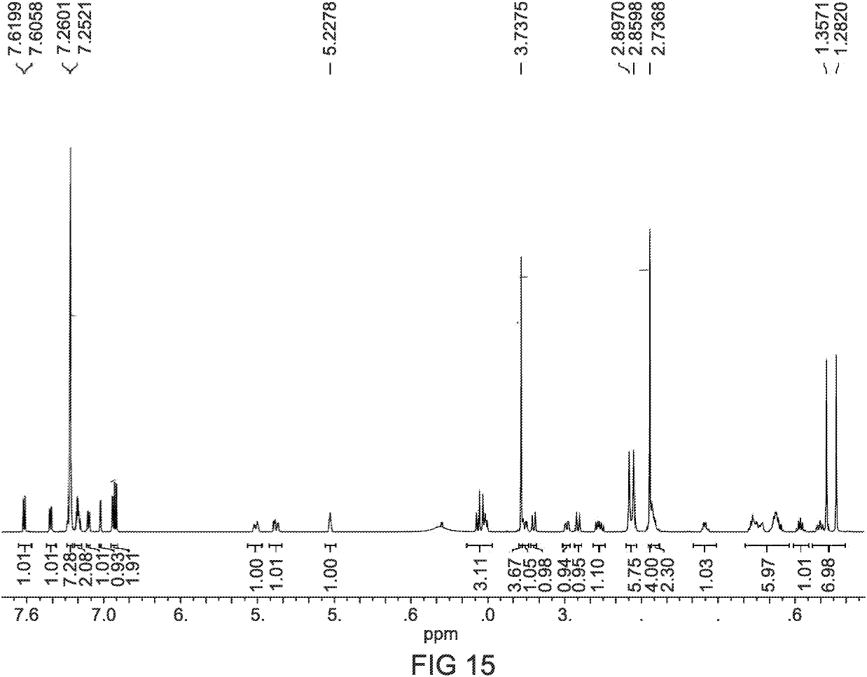

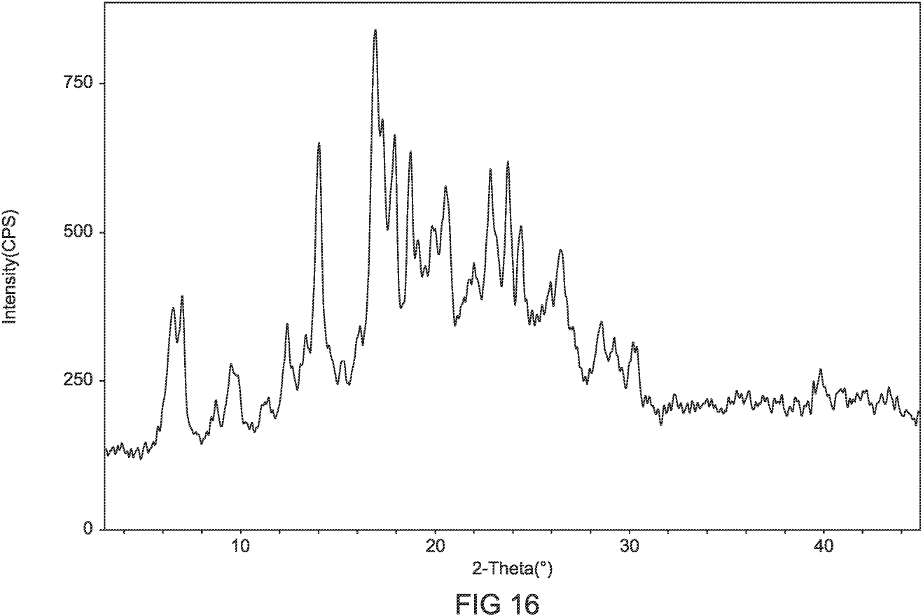

NMR spectrum (600 MHz in CDCl₃): FIG. 15.

### Formula I Imidazole Salt (Formula IC)

Formula I (168.0 mg (0.25 mmol, 1.0 eq.) amorphous) was added to a 25 mL vial. Ethyl acetate (4.0 mL) was added to give a clear solution. 18.9 mg of imidazole (0.275 mmol, 1.1 eq.) was added. The mixture was stirred for 5 minutes to give a clear solution. The mixture was continuously stirred overnight to give a slurry. The mixture was filtered and the cake was dried at room temperature under vacuum overnight to yield 118.0 mg (63.8%) of the imidazole salt of Formula I.

XRPD: FIG. 16.

DSC: FIG. 17.

TGA: FIG. 18

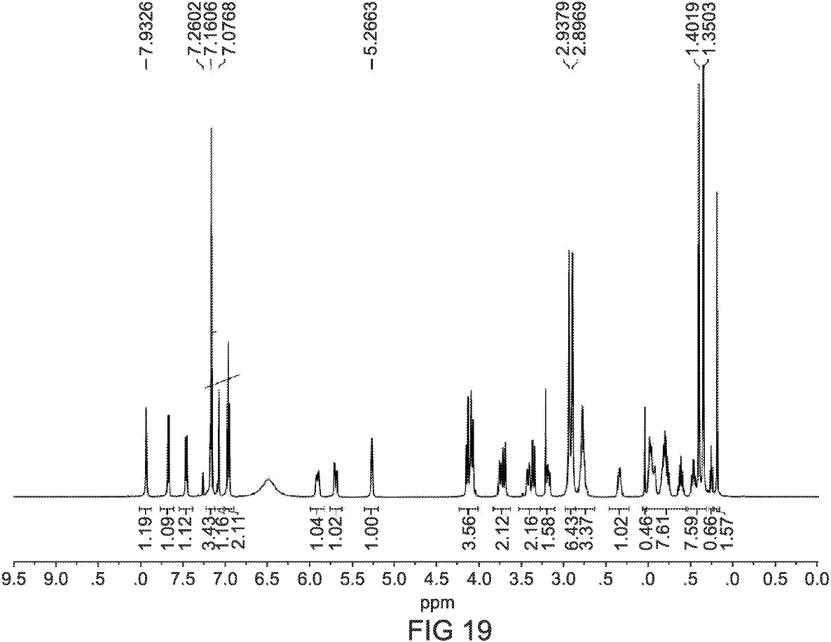

NMR spectrum (600 MHz in CDCl₃): FIG. 19.

### Formula I Piperazine Salt - (Form 1)

Formula I (168.0 mg (0.25 mmol, 1.0 eq.) amorphous) was added to a 25 mL vial. Ethyl acetate (4.0 mL) was added to give a clear solution. 23.2 mg of piperazine (0.275 mmol, 1.1 eq.) was added. The mixture was stirred for 5 minutes to give a clear solution. The mixture was continuously stirred overnight to give a slurry. The mixture was filtered and the cake was dried at room temperature under vacuum overnight to yield 100.5 mg (52.6%) of the piperazine salt of Formula I.

XRPD: FIG. 20.

DSC: FIG. 21.

TGA: FIG. 22.

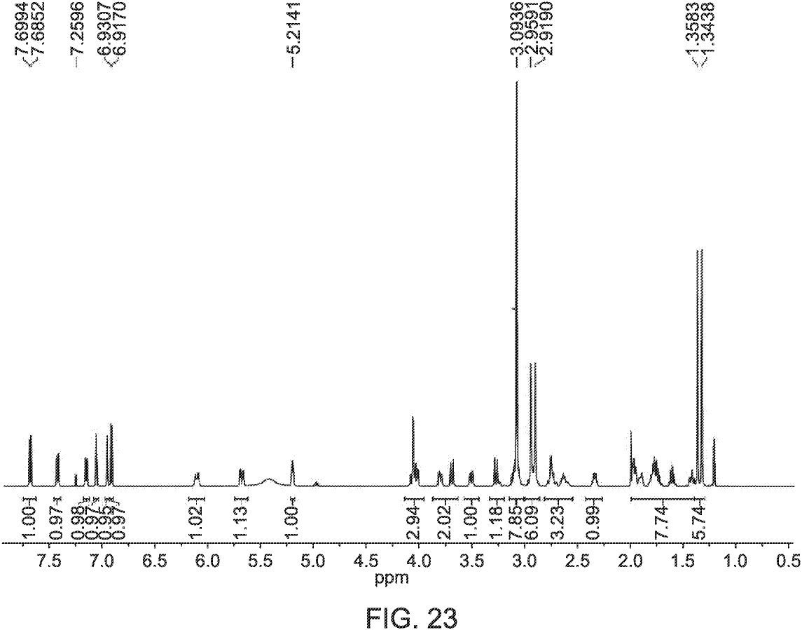

NMR spectrum (600 MHz in CDCl₃): FIG. 23.

### Formula I Piperazine Salt - (Form 2)

Formula I (25.0 mg; 0.037 mmol) was added to a 4 mL vial. 0.5 mL of acetonitrile was added and the mixture was stirred for 30 minutes. Piperazine (0.056 mmol, 1.50 eq.) was added and the mixture was stirred for 2 hrs, and then at 50° C. for 2 hrs. The mixture was cooled and then stirred at room temperature overnight, and then filtered to give the Formula I piperazine salt.

XRPD: FIG. 20A.

DSC: FIG. 21A.

### Formula I Piperazine Salt - (Form 3)

Formula I (25.0 mg; 0.037 mmol) was added to a 4 mL vial. 0.5 mL of methanol was added and the mixture was stirred for 30 minutes. Piperazine (0.056 mmol, 1.50 eq.) was added and the mixture was stirred for 2 hrs, and then at 50° C. for 2 hrs. The mixture was cooled and then stirred at room temperature overnight, and then filtered to give the Formula I piperazine salt.

XRPD: FIG. 20B.

### Formula I Piperazine Salt

Formula I (25.0 mg; 0.037 mmol) was added to a 4 mL vial. 0.5 mL of THF/methanol was added and the mixture was stirred for 30 minutes. Piperazine (0.056 mmol, 1.50 eq.) was added and the mixture was stirred for 2 hrs, and then at 50° C. for 2 hrs. The mixture was cooled and then stirred at room temperature overnight, and then filtered to give the Formula I piperazine salt.

### Formula I Piperidine Salt (Form 1)

Formula I (168.0 mg (0.25 mmol, 1.0 eq.) amorphous) was added to a 25 mL vial. Ethyl acetate (4.0 mL) was added to give a clear solution. 23.4 mg 4.5 mg of piperidine (0.275 mmol, 1.1 eq.) was added. The mixture was stirred for 5 minutes to give a clear solution. The mixture was continuously stirred overnight to give a slurry. The mixture was filtered and the cake was dried at room temperature under vacuum overnight to yield 110.8 mg (64.2%) of the piperidine salt of Formula I.

XRPD: FIG. 24.

DSC: FIG. 25.

TGA: FIG. 26.

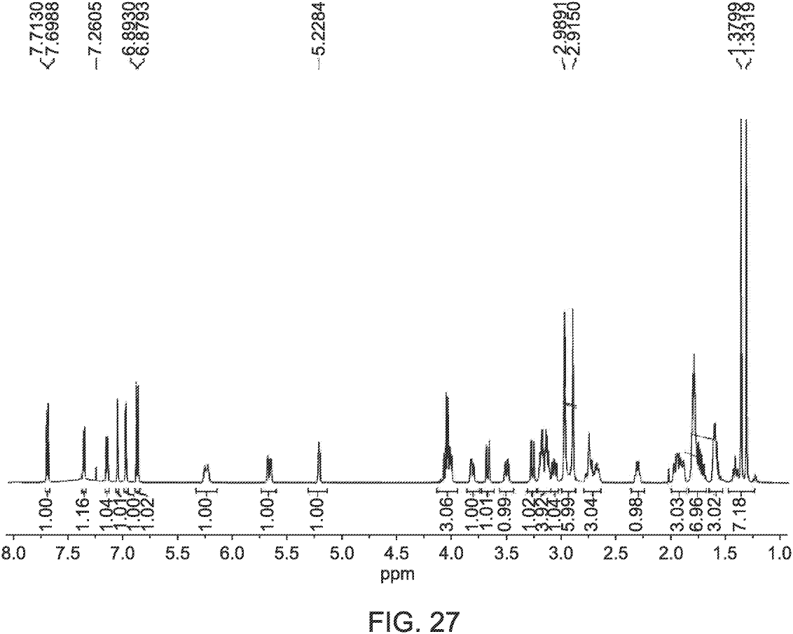

NMR spectrum (600 MHz in CDCl₃): FIG. 27.

### Formula I- Piperidine Salt - Method 2

The Formula I piperidine salt also was prepared by the reaction of Formula I free acid with 2.0 eq. of piperidine in IPA/MeOH.

### Formula I- Piperidine Salt - (Form 2)

The Formula I piperidine salt also was prepared by the reaction of Formula I free acid with piperidine in THF/MeOH.

XRPD: FIG. 24A.

### Formula I- Ethylenediamine Salt - (Form 1)

A mixture of Formula I free acid (1.0 eq.) and ethylenediamine (2.0 eq.) was stirred in isopropanol/MeOH to give crystalline solid ethylenediamine salt.

XRPD: FIG. 32.

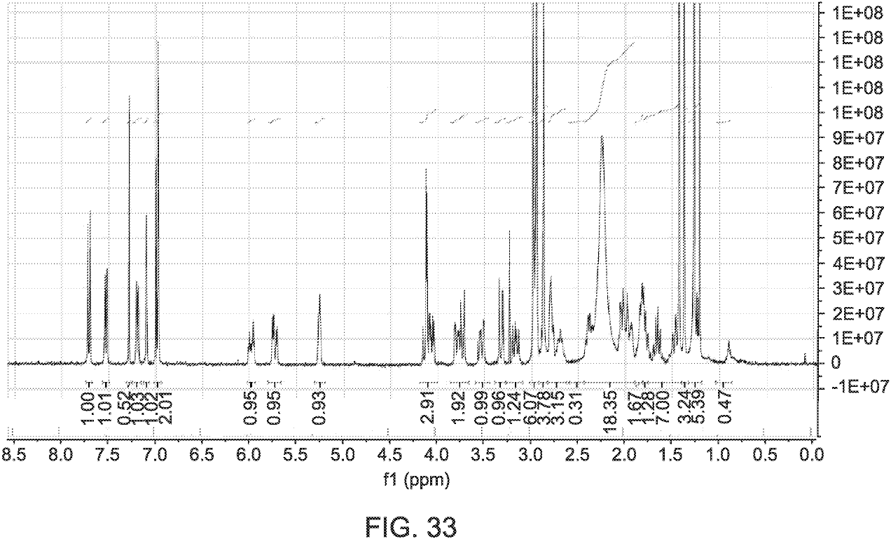

NMR Spectrum: FIG. 33.

### Formula I- Ethylenediamine Salt - (Form 2)

A mixture of Formula I free acid (1.0 eq.) and ethylenediamine (1.25 eq.) was stirred in THF/MeOH (1:5 mL) to give crystalline solid ethylenediamine salt.

XRPD: FIG. 32A.

### Formula I 4-((2-aminoethyl)amino)-4-methylpentan-2-one Salt

Formula I (168.0 mg (0.25 mmol, 1.0 eq.) amorphous) was added to a 25 mL vial. Ethyl acetate (4.0 mL) was added to give a clear solution. 275 µl of 1.0 M ethylene diamine in acetone (0.275 mmol, 1.1 eq.) was added. The mixture was stirred for 5 minutes to give a clear solution. The mixture was continuously stirred overnight to give a slurry. The mixture was filtered and the cake was dried at room temperature under vacuum overnight to yield 102.2 mg (63.8%) of the 4-((2-aminoethyl)amino)-4-methylpentan-2-one salt of Formula I.

It is believed that 4-((2-aminoethyl)amino)-4-methylpentan-2-one is formed in suit by reaction of ethylenediamine with acetone.

XRPD: FIG. 34.

DSC: FIG. 35.

TGA: FIG. 36.

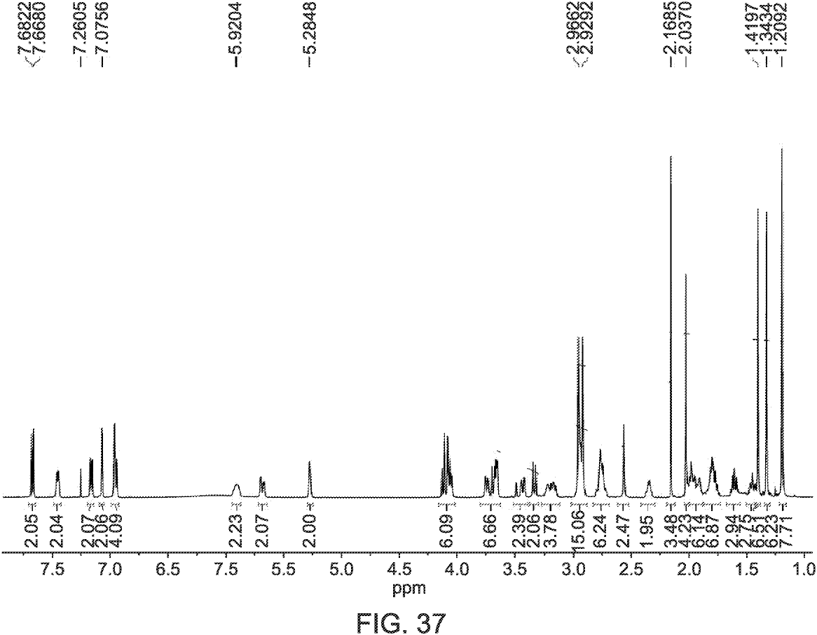

NMR spectrum (600 MHz in CDCl₃): FIG. 37.

### Formula I - Potassium Salt

The Formula I potassium salt was prepared by the reaction of Formula I free acid with potassium hydroxide (2 M in water, 2.0 eq.) in ethanol.

XRPD: FIG. 28.

DSC: FIG. 29.

The Formula I potassium salt also was prepared by the reaction of Formula I free acid with potassium hydroxide (2 M in water, 2.0 eq.) in isopropanol.

### Formula I - (S)-(-)-α-methylbenzylamine Salt

The Formula I-(S)-(-)-α-methylbenzylamine salt was prepared by the reaction of Formula I free acid with (S)-(-)-α-methylbenzylamine (1.5 eq.) in THF/methanol.

XRPD: FIG. 30.

DSC: FIG. 31.

### Instrument Methods

### X-Ray Powder Diffraction (XRPD)

XRPD patterns can be collected with a PANalytical X′Pert PRO MPD diffractometer using an incident beam of Cu radiation produced using an Optix long, fine-focus source. An elliptically graded multilayer mirror is used to focus Cu Kα X-rays through the specimen and onto the detector. Prior to the analysis, a silicon specimen (NIST SRM 640e) is analyzed to verify the observed position of the Si 111 peak is consistent with the NIST-certified position. A specimen of the sample is sandwiched between 3-µm-thick films and analyzed in transmission geometry. A beam-stop, short antiscatter extension, and antiscatter knife edge is used to minimize the background generated by air. Soller slits for the incident and diffracted beams are used to minimize broadening from axial divergence. Diffraction patterns are collected using a scanning position-sensitive detector (X′Celerator) located 240 mm from the specimen and Data Collector software v. 2.2b.

XRPD patterns also can be collected with a Rigaku MiniFlex X-ray Powder Diffractometer (XRPD) instrument. X-ray radiation is from Copper (Cu) at 1.54056 Å with K_{b} filter. X-ray power: 30 KV, 15 mA.

### Thermogravimetric Analysis (TGA) and Differential Scanning Calorimetry (DSC)

Thermal analysis can be performed using a Mettler Toledo TGA/DSC3+ analyzer. Temperature calibration is performed using phenyl salicylate, indium, tin, and zinc. The sample is placed in an aluminum pan. The sample is sealed, the lid pierced, then inserted into the TG furnace. The furnace is heated under nitrogen.

DSC can also be obtained using a TA Instrument Differential Scanning Calorimetry, Model Q20 with autosampler, using a scan rate of 10° C./min, and nitrogen gas flow at 50 mL/min.

TGA can be collected using a TGA Q500 by TA Instruments using a scan rate of 20° C. per minute.

### Dynamic Vapor Sorption (DVS)

The dynamic vapor sorption experiments can be done with a VTI SGA-Cx100 Symmetric Vapor Sorption Analyzer. The moisture uptake profile is completed in three cycles of 10% RH increments with adsorption from 5% to 95% RH, followed by desorption of 10% increments from 95% to 5%. The equilibration criteria are 0.0050 wt% in 5 minutes with a maximum equilibration time of 180 minutes. All adsorption and desorption are performed at room temperature (23-25° C.). No pre-drying step is applied for the samples.

### Biological Assays

### Cell Free Mcl-1:Bim Affinity Assay (Mcl-1 Bim)

The binding affinity of each compound was measured via a fluorescence polarization competition assay, in which the compound competes for the same binding site with the ligand, and thus leads to a dose-dependent anisotropy reduction. The tracer ligand utilized was a fluorescein isothiocyanate labelled peptide (FITC-ARIAQELRRIGDEFNETYTR) derived from Bim (GenScript).

The assay was carried out in black half-area 96-well NBS plate (Coming), containing 15 nM of MCL-1 (BPS Bioscience), 5 nM of FITC-Bim and 3-fold serial diluted test compounds in a total volume of 50 µL of assay buffer (20 mM HEPES, 50 mM NaCl, 0.002% Tween 20, 1 mM TCEP, and 1% DMSO). The reaction plate was incubated for 1 hour at room temperature. The change of anisotropy is measured with an Envision multimode plate reader (PerkinElmer) at emission wavelength 535 nm. Fluorescence polarization was calculated in mP unit and the percentage inhibition was calculated by % inhibition = 100x(mP_{DMSO}-mP)/(mP_{DMSO}-mP_{PC}), in which mP_{DMSO} is the DMSO control, and mP_{PC} is the positive control. IC₅₀ values were determined from a 10-point dose response curve by fitting the percent inhibition against compound concentration using the GraphPad Prism software. The inhibition constant Kᵢ was subsequently calculated according to the Nikolovska-Coleska’s equation (Anal. Biochem., 2004, 332, 261),

$${K}_{i} = \frac{{\left[I\right]}_{50}}{\frac{{\left[L\right]}_{50}}{{K}_{d}} + \frac{{\left[P\right]}_{0}}{{K}_{d}} + 1}$$

where [I]₅₀ is the concentration of the free inhibitor at 50% inhibition, [L]₅₀ is the concentration of the free labeled ligand at 50% inhibition, [P]₀ is the concentration of the free protein at 0% inhibition, and K_{d} is the dissociation constant of the protein-ligand complex. See Table A.

### Caspase 3/7 Activity Assay

Dispense 10 µL aliquot of prepared H929 cells (1:1 ratio of cells:Trypan Blue (#1450013, Bio-Rad)) onto cell counting slide (#145-0011, Bio-Rad) and obtain cell density and cell viability using cell counter (TC20, Bio-Rad). Remove appropriate volume of resuspended cells from culture flask to accommodate 2000 cells/well @ 5 µL/well. Transfer H929 cells to 50 mL conical (#430290, Coming) for each of the FBS concentration to be assayed (10%, 0.1%). Spin down at 1000 rpm for 5 min. using tabletop centrifuge (SPINCHRON 15, Beckman). Discard supernatant and resuspend cell pellet in modified RPMI 1640 (#10-040-CV, Coming) cell culture media containing sodium pyruvate (100 mM) (#25-000-CL, Corning), HEPES buffer (1 M) (#25-060-CL, Corning) and glucose (200 g/L) (A24940-01, Gibco) with appropriate FBS (F2422-500ML, Sigma) concentration to a cell density of 400,000 cells/mL. Dispense 5 µL of resuspended H929 cells per well in 384-well small volume TC treated plate (#784080, Greiner Bio-one) using standard cassette (#50950372, Thermo Scientific) on Multidrop Combi (#5840310, Thermo Scientific) in laminar flow cabinet. Dispense compounds onto plates using digital liquid dispenser (D300E, Tecan). Incubate plates in humidified tissue culture incubator @ 37° C. for 4 hours. Add 5 µL of prepared Caspase-Glo® 3/7 detection buffer (G8093, Promega) to each well of 384-well plate using small tube cassette (#24073295, Thermo Scientific) on Combi multi-drop, incubate @ RT for 30-60 min. Read plates with microplate reader (PheraStar, BMG Labtech) using 384 well luminescence mode.

### Cell Viability Assay (H929 10 FBS)

Dispense 10 µL aliquot of prepared H929 cells (1:1 ratio of cells:Trypan Blue (#1450013, Bio-Rad)) onto cell counting slide (#145-0011, Bio-Rad) and obtain cell density and cell viability using cell counter (TC20, Bio-Rad). Remove appropriate volume of resuspended cells from culture flask to accommodate 4000 cells/well @ 10 µL/well. Transfer H929 cells to 50 mL conical (#430290, Corning). Spin down at 1000 rpm for 5 min using tabletop centrifuge (SPINCHRON 15, Beckman). Discard supernatant and resuspend cell pellet in modified RPMI 1640 (#10-040-CV, Coming) cell culture media containing 10% FBS (F2422-500 ML, Sigma), sodium pyruvate (100 mM) (#25-000-CL, Corning), HEPES buffer (1 M) (#25-060-CL, Coming) and glucose (200 g/L) (A24940-01, Gibco) to a cell density of 400,000 cells/mL. Dispense 10 µL of resuspended H929 cells per well in 384-well small volume TC treated plate (#784080, Greiner Bio-one) using standard cassette (#50950372, Thermo Scientific) on Multi-drop Combi (#5840310, Thermo Scientific) in laminar flow cabinet. Dispense compounds onto plates using digital liquid dispenser (D300E, Tecan). Incubate plates in humidified tissue culture incubator @ 37° C. for 24 hours. Add 10 µL of prepared CellTiTer-Glo® detection buffer (G7570, Promega) or ATPlite 1Step detection reagent (#6016731, Perkin Elmer) to each well of 384-well plate using small tube cassette (#24073295, Thermo Scientific) on Combi multi drop, incubate @ RT for 30-60 min. Read plates with microplate reader (PheraStar, BMG Labtech) using 384 well luminescence mode.

### Cytotoxicity Studies in NCI-H929 Cells

Cytotoxicity studies were conducted in NCI-H929 multiple myeloma cell line. Cells were maintained in RPMI 1640 (Coming Cellgro, Catalog #: 10-040-CV) supplemented with 10% v/v FBS (GE Healthcare, Catalog #: SH30910.03), 10 mM HEPES (Corning, Catalog #: 25-060-CI), 1 mM sodium pyruvate (Coming Cellgro, Catalog #: 25-000-CI and 2500 mg/L glucose (Gibco, Catalog #: A24940-01). Cells were seeded in 96-well plates at a density of 75000 cells/well. Compounds dissolved in DMSO were plated in duplicate using a digital dispenser (Tecan D300E) and tested on a 9-point 3-fold serial dilution. Cells were incubated for 24 hr in a 37° C. incubator at 5% CO₂. Cell viability was measured using the Cell Counting Kit-8 (CCK-8, Jojindo, CK04-13) as per manufacturer’s instructions. Cells were incubated for 4 hours at 37° C. 5% CO₂ following addition of reagent and OD₄₅₀ values were measured with a microplate reader (iMark microplate reader, Bio-Rad). Background from media only wells were averaged and subtracted from all readings. OD₄₅₀ values were then normalized to DMSO controls to obtain percentage of viable cells, relative to DMSO vehicle control and plotted in Graphpad Prism ([Inhibitor] vs. normalized response - Variable slope; equation: Y=100 / (1 + (X^HillSlope) /(IC₅₀^HillSlope)) ) to determine IC₅₀ values (the concentration of compound inhibiting half of the maximal activity).

TABLE A

Cell free Mcl-1:Bim affinity assay (Mcl-1 Bim) and Cell viability assay (H929_10FBS)

| | | |
|---|---|---|
| Ex No. | BIM_ Ki (nM) | H929_10FB S IC₅₀ (nM) |
| 34 (Formula I) | +++ | ### |

+++ Kᵢ < 1 nM; ++ Kᵢ = 1 nM - 100 nM; ### IC₅₀ < 500 nM; ## IC₅₀ < 1000 nM; # IC₅₀ > 1000 nM; NT = not tested.

What is claimed:

1. A crystalline form of the compound of Formula I: 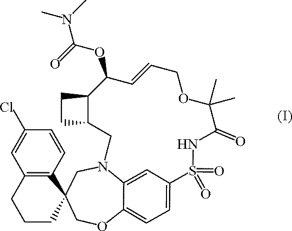 [SMILES: CN(C)C(=O)O[C@H]1/C=C/COC(C)(C)C(=O)NS(=O)(=O)c2ccc3c(c2)N(C[C@@H]2CC[C@H]21)C[C@@]1(CCCc2cc(Cl)ccc21)CO3]
.

2. The crystalline form of claim 1, wherein the crystalline form is Formula I-Form 1.

3. The crystalline form of either claim 1 or claim 2, characterized by an X-ray powder diffraction pattern substantially as shown in FIG. 1.

4. The crystalline form of any one of claims 1-3, characterized by an X-ray powder diffraction pattern comprising peaks at 11.2, 13.9, 17.1, 17.7, and 20.8 degrees ± 0.2 degrees 2-theta, on the 2-theta scale with lambda = 1.54 angstroms (Cu Kα).

5. The crystalline form of any one of the preceding claims, characterized by an X-ray powder diffraction pattern comprising peaks at 13.9, 17.1, 17.7, 20.8, and 21.9 degrees ± 0.2 degrees 2-theta, on the 2-theta scale with lambda = 1.54 angstroms (Cu Kα).

6. The crystalline form of any one of the preceding claims, characterized by an X-ray powder diffraction pattern comprising peaks at 11.2, 13.9, 17.1, 17.7, 20.8, 21.9, and 25.0 degrees ± 0.2 degree 2-theta, on the 2-theta scale with lambda = 1.54 angstroms (Cu Kα).

7. The crystalline form of any one of the preceding claims, characterized by an X-ray powder diffraction pattern comprising peaks at 9.4, 11.2, 13.9, 17.1, 17.7, 20.8, 21.9, 25.0, and 27.8 degrees ± 0.2 degree 2-theta, on the 2-theta scale with lambda = 1.54 angstroms (Cu Kα).

8. The crystalline form of any one of the preceding claims, characterized by an X-ray powder diffraction pattern comprising peaks at three or more of 9.4, 11.2, 13.9, 17.1, 17.7, 20.8, 21.9, 25.0, and 27.8 degrees ± 0.2 degrees 2-theta, on the 2-theta scale with lambda = 1.54 angstroms (Cu Kα).

9. The crystalline form of any one of the preceding claims, characterized by a differential scanning calorimetry (DSC) thermogram substantially as shown in FIG. 2 when heated at a rate of 10° C./min.

10. The crystalline form of any one of the preceding claims, characterized by a differential scanning calorimetry (DSC) thermogram comprising an endothermic peak at about 81° C. when heated at a rate of 10° C./min.

11. The crystalline form of any one of the preceding claims, characterized by a thermogravimetric analysis profile substantially as shown in FIG. 3 when heated at a rate of 20° C./min.

12. The crystalline form of claim 1, wherein the crystalline form is Formula I-Form II.

13. The crystalline form of either claim 1 or claim 12, characterized by an X-ray powder diffraction pattern substantially as shown in FIG. 6.

14. The crystalline form of any one of claims 1, or 12-13, characterized by an X-ray powder diffraction pattern comprising peaks at 9.2, 21.7, and 30.5 degrees ± 0.2 degrees 2-theta, on the 2-theta scale with lambda =1.54 angstroms (Cu Kα).

15. The crystalline form of any one of claims 1, or 12-14, characterized by an X-ray powder diffraction pattern comprising peaks at 17.4, 18.1, 19.3, 19.8, and 30.5 degrees ± 0.2 degrees 2-theta, on the 2-theta scale with lambda = 1.54 angstroms (Cu Kα).

16. The crystalline form of any one of claims 1, or 12-15, characterized by an X-ray powder diffraction pattern comprising peaks at 12.6, 17.4, 18.1, 19.3, 19.8, 21.7, 28.6, and 30.5 degrees ± 0.2 degree 2-theta, on the 2-theta scale with lambda = 1.54 angstroms (Cu Kα).

17. The crystalline form of any one of claims 1, or 12-16, characterized by an X-ray powder diffraction pattern comprising peaks at 9.2, 12.6, 17.4, 18.1, 19.3, 19.8, 21.7, 28.6, 30.5, and 34.9 degrees ± 0.2 degree 2-theta, on the 2-theta scale with lambda = 1.54 angstroms (Cu Kα).

18. The crystalline form of any one of claims 1, or 12-17, characterized by an X-ray powder diffraction pattern comprising peaks at one or more of 9.2, 12.6, 17.4, 18.1, 19.3, 19.8, 21.7, 28.6, 30.5, and 34.9 degrees ± 0.2 degrees 2-theta, on the 2-theta scale with lambda = 1.54 angstroms (Cu Kα).

19. The crystalline form of any one of claims 1, or 12-18, characterized by a differential scanning calorimetry (DSC) thermogram substantially as shown in FIG. 7 when heated at a rate of 10° C./min.

20. The crystalline form of any one of claims 1, or 12-19, characterized by a differential scanning calorimetry (DSC) thermogram comprising an endothermic peak at about 68° C. when heated at a rate of 10° C./min.

21. The crystalline form of any one of claims 1, or 12-20, characterized by a differential scanning calorimetry (DSC) thermogram comprising an endothermic peak at about 92° C. when heated at a rate of 10° C./min.

22. A pharmaceutically acceptable salt of a compound of Formula I 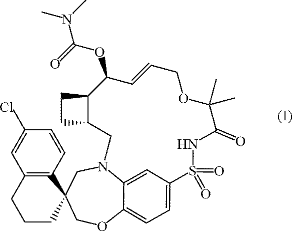 [SMILES: CN(C)C(=O)O[C@H]1/C=C/COC(C)(C)C(=O)NS(=O)(=O)c2ccc3c(c2)N(C[C@@H]2CC[C@H]21)C[C@@]1(CCCc2cc(Cl)ccc21)CO3]
.

23. The pharmaceutically acceptable salt of claim 22, wherein the salt is the choline salt having Formula IA 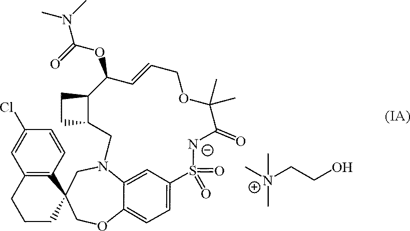
.

24. A crystalline form of the pharmaceutically acceptable salt of claim 23.

25. The crystalline form of claim 24, characterized by an X-ray powder diffraction pattern substantially as shown in FIG. 8.

26. The crystalline form of either claim 24 or claim 25, characterized by an X-ray powder diffraction pattern comprising peaks at 19.4, and 20.0 degrees ± 0.2 degrees 2-theta, on the 2-theta scale with lambda = 1.54 angstroms (Cu Kα).

27. The crystalline form of either claim 24 or claim 25, characterized by an X-ray powder diffraction pattern comprising peaks at 18.5, 19.4, 20.0, and 22.6 degrees ± 0.2 degrees 2-theta, on the 2-theta scale with lambda =1.54 angstroms (Cu Kα).

28. The crystalline form of either claim 24 or claim 25, characterized by an X-ray powder diffraction pattern comprising peaks at 13.3, 18.5, 19.4, 20.0, 22.6, and 24.7 degrees ± 0.2 degree 2-theta, on the 2-theta scale with lambda = 1.54 angstroms (Cu Kα).

29. The crystalline form of either claim 24 or claim 25, characterized by an X-ray powder diffraction pattern comprising peaks at 9.9, 13.3, 18.5, 19.4, 20.0, 22.6, and 24.7 degrees ± 0.2 degree 2-theta, on the 2-theta scale with lambda = 1.54 angstroms (Cu Kα).

30. The crystalline form of either claim 24 or claim 25, characterized by an X-ray powder diffraction pattern comprising peaks at one or more of 9.9, 13.3, 18.5, 19.4, 20.0, 22.6, and 24.7 degrees ± 0.2 degrees 2-theta, on the 2-theta scale with lambda = 1.54 angstroms (Cu Kα).

31. The crystalline form of any one of claims 24 to 30, characterized by a differential scanning calorimetry (DSC) thermogram substantially as shown in FIG. 9 when heated at a rate of 10° C./min.

32. The crystalline form of any one of claims 24 to 31, characterized by a differential scanning calorimetry (DSC) thermogram comprising an endothermic peak at about 158° C. when heated at a rate of 10° C./min.

33. The crystalline form of any one of claims 24 to 32, characterized by a thermogravimetric analysis profile substantially as shown in FIG. 10 when heated at a rate of 20° C./min.

34. The pharmaceutically acceptable salt of claim 22, wherein the salt is the benzathine salt having Formula IB 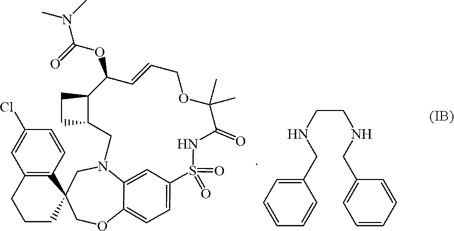
.

35. A crystalline form of the pharmaceutically acceptable salt of claim 34.

36. The crystalline form of claim 35, characterized by an X-ray powder diffraction pattern substantially as shown in FIG. 12.

37. The crystalline form of either claim 35 or claim 36, characterized by an X-ray powder diffraction pattern comprising peaks at 5.8, and 18.2 degrees ± 0.2 degrees 2-theta, on the 2-theta scale with lambda = 1.54 angstroms (Cu Kα).

38. The crystalline form of either claim 35 or claim 36, characterized by an X-ray powder diffraction pattern comprising peaks at 5.8, 16.6, 18.2, and 20.7 degrees ± 0.2 degrees 2-theta, on the 2-theta scale with lambda = 1.54 angstroms (Cu Kα).

39. The crystalline form of either claim 35 or claim 36, characterized by an X-ray powder diffraction pattern comprising peaks at 5.8, 12.6, 16.6, 18.2, and 20.7 degrees ± 0.2 degree 2-theta, on the 2-theta scale with lambda = 1.54 angstroms (Cu Kα).

40. The crystalline form of either claim 35 or claim 36, characterized by an X-ray powder diffraction pattern comprising peaks at 5.8, 12.6, 16.6, 18.2, 20.7, and 22.2 degrees ± 0.2 degree 2-theta, on the 2-theta scale with lambda = 1.54 angstroms (Cu Kα).

41. The crystalline form of either claim 35 or claim 36, characterized by an X-ray powder diffraction pattern comprising peaks at one or more of 5.8, 12.6, 16.6, 18.2, 20.7, and 22.2 degrees ± 0.2 degrees 2-theta, on the 2-theta scale with lambda = 1.54 angstroms (Cu Kα).

42. The crystalline form of any one of claims 35 to 41, characterized by a differential scanning calorimetry (DSC) thermogram substantially as shown in FIG. 13 when heated at a rate of 10° C./min.

43. The crystalline form of any one of claims 35 to 42, characterized by a differential scanning calorimetry (DSC) thermogram comprising an endothermic peak at about 112° C. when heated at a rate of 10° C./min.

44. The crystalline form of any one of claims 35 to 43, characterized by a thermogravimetric analysis profile substantially as shown in FIG. 14 when heated at a rate of 20° C./min.

45. The pharmaceutically acceptable salt of claim 22, wherein the salt is the imidazole salt having Formula IC: 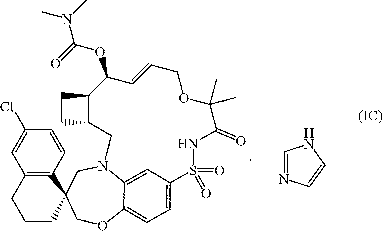
.

46. A crystalline form of the pharmaceutically acceptable salt of claim 45.

47. The crystalline form of claim 46, characterized by an X-ray powder diffraction pattern substantially as shown in FIG. 16.

48. The crystalline form of either claim 46 or claim 47, characterized by an X-ray powder diffraction pattern comprising peaks at 14.1 and 17.0 degrees ± 0.2 degrees 2-theta, on the 2-theta scale with lambda = 1.54 angstroms (Cu Kα).

49. The crystalline form of either claim 46 or claim 47, characterized by an X-ray powder diffraction pattern comprising peaks at 14.1, 17.0, 17.9, 18.8, and 20.6 degrees ± 0.2 degrees 2-theta, on the 2-theta scale with lambda = 1.54 angstroms (Cu Kα).

50. The crystalline form of either claim 46 or claim 47, characterized by an X-ray powder diffraction pattern comprising peaks at 14.1, 17.0, 17.9, 18.8, 20.6, 22.0, 22.9, and 23.8 degrees ± 0.2 degree 2-theta, on the 2-theta scale with lambda = 1.54 angstroms (Cu Kα).

51. The crystalline form of either claim 46 or claim 47, characterized by an X-ray powder diffraction pattern comprising peaks at 6.5, 7.0, 14.1, 17.0, 17.9, 18.8, 20.6, 22.0, 22.9, and 23.8 degrees ± 0.2 degree 2-theta, on the 2-theta scale with lambda = 1.54 angstroms (Cu Kα).

52. The crystalline form of either claim 46 or claim 47, characterized by an X-ray powder diffraction pattern comprising peaks at one or more of 6.5, 7.0, 14.1, 17.0, 17.9, 18.8, 20.6, 22.0, 22.9, 23.8, 24.4, and 26.5 degrees ± 0.2 degrees 2-theta, on the 2-theta scale with lambda = 1.54 angstroms (Cu Kα).

53. The crystalline form of any one of claims 46 to 52, characterized by a differential scanning calorimetry (DSC) thermogram substantially as shown in FIG. 17 when heated at a rate of 10° C./min.

54. The crystalline form of any one of claims 46 to 53, characterized by a differential scanning calorimetry (DSC) thermogram comprising an endothermic peak at about 135° C. when heated at a rate of 10° C./min.

55. The crystalline form of any one of claims 46 to 54, characterized by a thermogravimetric analysis profile substantially as shown in FIG. 18 when heated at a rate of 20° C./min.

56. The pharmaceutically acceptable salt of claim 22, wherein the salt is the piperazine salt having Formula ID 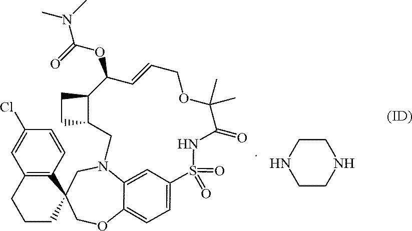
.

57. A crystalline form of the pharmaceutically acceptable salt of claim 56.

58. The crystalline form of claim 57, wherein said form is crystalline Form 1.

59. The crystalline form of claim 58, characterized by an X-ray powder diffraction pattern substantially as shown in FIG. 20.

60. The crystalline form of either claim 58 or claim 59, characterized by an X-ray powder diffraction pattern comprising peaks at 7.1, 12.2, and 14.8 degrees ± 0.2 degrees 2-theta, on the 2-theta scale with lambda = 1.54 angstroms (Cu Kα).

61. The crystalline form of either claim 58 or claim 59, characterized by an X-ray powder diffraction pattern comprising peaks at 7.1, 12.2, 14.8, 16.0, 17.9, and 19.7 degrees ± 0.2 degrees 2-theta, on the 2-theta scale with lambda = 1.54 angstroms (Cu Kα).

62. The crystalline form of either claim 58 or claim 59, characterized by an X-ray powder diffraction pattern comprising peaks at 7.1, 12.2, 14.8, 16.0, 17.9, 19.7, and 20.5 degrees ± 0.2 degree 2-theta, on the 2-theta scale with lambda = 1.54 angstroms (Cu Kα).

63. The crystalline form of either claim 58 or claim 59, characterized by an X-ray powder diffraction pattern comprising peaks at 7.1, 12.2, 14.8, 16.0, 17.9, 19.7, 20.5, and 22.8 degrees ± 0.2 degree 2-theta, on the 2-theta scale with lambda = 1.54 angstroms (Cu Kα).

64. The crystalline form of either claim 58 or claim 59, characterized by an X-ray powder diffraction pattern comprising peaks at one or more of 7.1, 12.2, 14.8, 16.0, 17.9, 19.7, 20.5, and 22.8 degrees ± 0.2 degrees 2-theta, on the 2-theta scale with lambda = 1.54 angstroms (Cu Kα).

65. The crystalline form of any one of claims 58 to 64, characterized by a differential scanning calorimetry (DSC) thermogram substantially as shown in FIG. 21 when heated at a rate of 10° C./min.

66. The crystalline form of any one of claims 58 to 65, characterized by a differential scanning calorimetry (DSC) thermogram comprising an endothermic peak at about 160° C. when heated at a rate of 10° C./min.

67. The crystalline form of any one of claims 58 to 66, characterized by a thermogravimetric analysis profile substantially as shown in FIG. 22 when heated at a rate of 20° C./min.

68. The crystalline form of claim 57, wherein said form is crystalline Form 2.

69. The crystalline form of claim 68, characterized by an X-ray powder diffraction pattern substantially as shown in FIG. 20A.

70. The crystalline form of either claim 68 or claim 69, characterized by an X-ray powder diffraction pattern comprising peaks at 16.5 and 17.8 degrees ± 0.2 degrees 2-theta, on the 2-theta scale with lambda = 1.54 angstroms (Cu Kα).

71. The crystalline form of either claim 68 or claim 69, characterized by an X-ray powder diffraction pattern comprising peaks at 5.5, 6.2, 8.6, 14.0, 16.5, and 17.8 degrees ± 0.2 degrees 2-theta, on the 2-theta scale with lambda = 1.54 angstroms (Cu Kα).

72. The crystalline form of either claim 68 or claim 69, characterized by an X-ray powder diffraction pattern comprising peaks at 16.5, 17.8, 19.1, 20.5, 22.1, and 23.0 degrees ± 0.2 degree 2-theta, on the 2-theta scale with lambda = 1.54 angstroms (Cu Kα).

73. The crystalline form of either claim 68 or claim 69, characterized by an X-ray powder diffraction pattern comprising peaks at 5.5, 6.2, 8.6, 14.0, 16.5, 17.8, 19.1, 20.5, 22.1, and 23.0 degrees ± 0.2 degree 2-theta, on the 2-theta scale with lambda = 1.54 angstroms (Cu Kα).

74. The crystalline form of either claim 68 or claim 69, characterized by an X-ray powder diffraction pattern comprising peaks at one or more of 5.5, 6.2, 8.6, 14.0, 16.5, 17.8, 19.1, 20.5, 22.1, and 23.0 degrees ± 0.2 degrees 2-theta, on the 2-theta scale with lambda = 1.54 angstroms (Cu Kα).

75. The crystalline form of any one of claims 68 to 74, characterized by a differential scanning calorimetry (DSC) thermogram substantially as shown in FIG. 21A when heated at a rate of 10° C./min.

76. The crystalline form of any one of claims 68 to 75, characterized by a differential scanning calorimetry (DSC) thermogram comprising an endothermic peak at about 143° C. when heated at a rate of 10° C./min.

77. The crystalline form of claim 57, wherein said form is crystalline Form 3.

78. The crystalline form of claim 77, characterized by an X-ray powder diffraction pattern substantially as shown in FIG. 20B.

79. The crystalline form of either claim 77 or claim 78, characterized by an X-ray powder diffraction pattern comprising peaks at 18.5, 19.4, and 19.9 degrees ± 0.2 degrees 2-theta, on the 2-theta scale with lambda = 1.54 angstroms (Cu Kα).

80. The crystalline form of either claim 77 or claim 78, characterized by an X-ray powder diffraction pattern comprising peaks at 16.5, 16.9, 18.5, 19.4, 19.9, and 22.7 degrees ± 0.2 degrees 2-theta, on the 2-theta scale with lambda = 1.54 angstroms (Cu Kα).

81. The crystalline form of either claim 77 or claim 78, characterized by an X-ray powder diffraction pattern comprising peaks at 13.8, 16.5, 16.9, 18.5, 19.4, 19.9, and 22.7 degrees ± 0.2 degree 2-theta, on the 2-theta scale with lambda = 1.54 angstroms (Cu Kα).

82. The crystalline form of either claim 77 or claim 78, characterized by an X-ray powder diffraction pattern comprising peaks at 6.3, 6.7, 11.0, 11.6, 13.8, 16.5, 16.9, 18.5, 19.4, 19.9, and 22.7 degrees ± 0.2 degree 2-theta, on the 2-theta scale with lambda = 1.54 angstroms (Cu Kα).

83. The crystalline form of either claim 77 or claim 78, characterized by an X-ray powder diffraction pattern comprising peaks at one or more of 6.3, 6.7, 11.0, 11.6, 13.8, 16.5, 16.9, 18.5, 19.4, 19.9, and 22.7 degrees ± 0.2 degrees 2-theta, on the 2-theta scale with lambda = 1.54 angstroms (Cu Kα).

84. The pharmaceutically acceptable salt of claim 22, wherein the salt is the piperidine salt having Formula IE 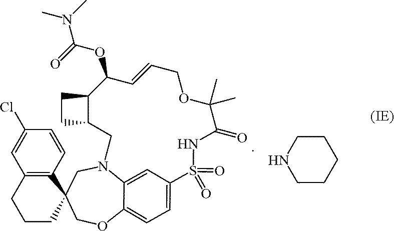
.

85. A crystalline form of the pharmaceutically acceptable salt of claim 84.

86. The crystalline form of claim 85, wherein said form is crystalline Form 1.

87. The crystalline form of claim 86, characterized by an X-ray powder diffraction pattern substantially as shown in FIG. 24.

88. The crystalline form of either claim 86 or claim 87, characterized by an X-ray powder diffraction pattern comprising peaks at 7.3 and 17.9 degrees ± 0.2 degrees 2-theta, on the 2-theta scale with lambda = 1.54 angstroms (Cu Kα).

89. The crystalline form of either claim 86 or claim 87, characterized by an X-ray powder diffraction pattern comprising peaks at 7.3, 12.2, 16.1, and 17.9 degrees ± 0.2 degrees 2-theta, on the 2-theta scale with lambda = 1.54 angstroms (Cu Kα).

90. The crystalline form of either claim 86 or claim 87, characterized by an X-ray powder diffraction pattern comprising peaks at 7.3, 12.2, 14.3, 14.8, 16.1, 17.9, and 19.8 degrees ± 0.2 degree 2-theta, on the 2-theta scale with lambda = 1.54 angstroms (Cu Kα).

91. The crystalline form of either claim 86 or claim 87, characterized by an X-ray powder diffraction pattern comprising peaks at 7.3, 12.2, 14.3, 14.8, 16.1, 17.9, 19.8, 20.6, and 22.9 degrees ± 0.2 degree 2-theta, on the 2-theta scale with lambda = 1.54 angstroms (Cu Kα).

92. The crystalline form of either claim 86 or claim 87, characterized by an X-ray powder diffraction pattern comprising peaks at one or more of 7.3, 12.2, 14.3, 14.8, 16.1, 17.9, 19.8, 20.6, and 22.9 degrees ± 0.2 degrees 2-theta, on the 2-theta scale with lambda = 1.54 angstroms (Cu Kα).

93. The crystalline form of any one of claims 86 to 92, characterized by a differential scanning calorimetry (DSC) thermogram substantially as shown in FIG. 25 when heated at a rate of 10° C./min.

94. The crystalline form of any one of claims 86 to 93, characterized by a differential scanning calorimetry (DSC) thermogram comprising an endothermic peak at about 174° C. when heated at a rate of 10° C./min.

95. The crystalline form of any one of claims 86 to 94, characterized by a thermogravimetric analysis profile substantially as shown in FIG. 26 when heated at a rate of 20° C./min.

96. The crystalline form of claim 85, wherein said form is crystalline Form 2.

97. The crystalline form of claim 96, characterized by an X-ray powder diffraction pattern substantially as shown in FIG. 24A.

98. The crystalline form of either claim 96 or claim 97, characterized by an X-ray powder diffraction pattern comprising a peak at 18.3 degrees ± 0.2 degrees 2-theta, on the 2-theta scale with lambda = 1.54 angstroms (Cu Kα).

99. The crystalline form of either claim 96 or claim 97, characterized by an X-ray powder diffraction pattern comprising peaks at 10.9, 16.8, and 18.3 degrees ± 0.2 degree 2-theta, on the 2-theta scale with lambda = 1.54 angstroms (Cu Kα).

100. The crystalline form of either claim 96 or claim 97, characterized by an X-ray powder diffraction pattern comprising peaks at one or more of 16.8, 18.3, and 20.7 degrees ± 0.2 degrees 2-theta, on the 2-theta scale with lambda = 1.54 angstroms (Cu Kα).

101. The pharmaceutically acceptable salt of claim 22, wherein the salt is the potassium salt having Formula IF 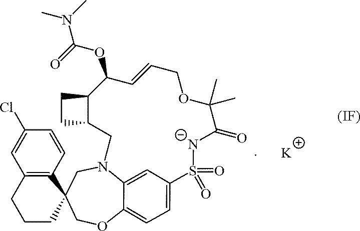
.

102. A crystalline form of the pharmaceutically acceptable salt of claim 101.

103. The crystalline form of claim 102, characterized by an X-ray powder diffraction pattern substantially as shown in FIG. 28.

104. The crystalline form of either claim 102 or claim 103, characterized by an X-ray powder diffraction pattern comprising peaks at 9.1, 10.4, 18.0, and 19.3 degrees ± 0.2 degrees 2-theta, on the 2-theta scale with lambda = 1.54 angstroms (Cu Kα).

105. The crystalline form of either claim 102 or claim 103, characterized by an X-ray powder diffraction pattern comprising peaks at 9.1, 10.4, 19.3, and 22.8 degrees ± 0.2 degrees 2-theta, on the 2-theta scale with lambda = 1.54 angstroms (Cu Kα).

106. The crystalline form of either claim 102 or claim 103, characterized by an X-ray powder diffraction pattern comprising peaks at 9.1, 10.4, 18.0, 19.3, 22.8, and 24.4 degrees ± 0.2 degree 2-theta, on the 2-theta scale with lambda = 1.54 angstroms (Cu Kα).

107. The crystalline form of either claim 102 or claim 103, characterized by an X-ray powder diffraction pattern comprising peaks at 9.1, 10.4, 12.5, 15.1, 18.0, 19.3, 22.8, and 24.4 degrees ± 0.2 degree 2-theta, on the 2-theta scale with lambda = 1.54 angstroms (Cu Kα).

108. The crystalline form of either claim 102 or claim 103, characterized by an X-ray powder diffraction pattern comprising peaks at one or more of 9.1, 10.4, 12.5, 15.1, 18.0, 19.3, 22.8, and 24.4 degrees ± 0.2 degrees 2-theta, on the 2-theta scale with lambda = 1.54 angstroms (Cu Kα).

109. The crystalline form of any one of claims 102 to 108, characterized by a differential scanning calorimetry (DSC) thermogram substantially as shown in FIG. 29 when heated at a rate of 10° C./min.

110. The crystalline form of any one of claims 102 to 109, characterized by a differential scanning calorimetry (DSC) thermogram comprising an endothermic peak at about 150° C. when heated at a rate of 10° C./min.

111. The pharmaceutically acceptable salt of claim 22, wherein the salt is the (S)-(-)-α-methylbenzylamine salt having Formula IG 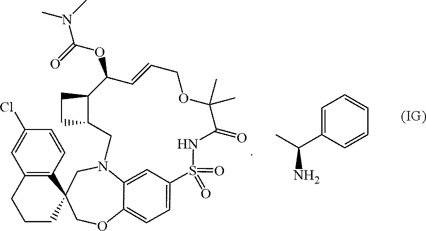
.

112. A crystalline form of the pharmaceutically acceptable salt of claim 111.

113. The crystalline form of claim 112, characterized by an X-ray powder diffraction pattern substantially as shown in FIG. 30.

114. The crystalline form of either claim 112 or claim 113, characterized by an X-ray powder diffraction pattern comprising a peak at 19.9 degrees ± 0.2 degrees 2-theta, on the 2-theta scale with lambda = 1.54 angstroms (Cu Kα).

115. The crystalline form of either claim 112 or claim 113, characterized by an X-ray powder diffraction pattern comprising a peak at 18.2 degrees ± 0.2 degrees 2-theta, on the 2-theta scale with lambda = 1.54 angstroms (Cu Kα).

116. The crystalline form of either claim 112 or claim 113, characterized by an X-ray powder diffraction pattern comprising peaks at 18.2 and 19.9 degrees ± 0.2 degree 2-theta, on the 2-theta scale with lambda = 1.54 angstroms (Cu Kα).

117. The crystalline form of any one of claims 112 to 116, characterized by a differential scanning calorimetry (DSC) thermogram substantially as shown in FIG. 31 when heated at a rate of 10° C./min.

118. The crystalline form of any one of claims 112 to 117, characterized by a differential scanning calorimetry (DSC) thermogram comprising an endothermic peak at about 75° C. when heated at a rate of 10° C./min.

119. The crystalline form of any one of claims 112 to 118, characterized by a differential scanning calorimetry (DSC) thermogram comprising an endothermic peak at about 114° C. when heated at a rate of 10° C./min.

120. The pharmaceutically acceptable salt of claim 22, wherein the salt is the ethylene diamine salt having Formula IH 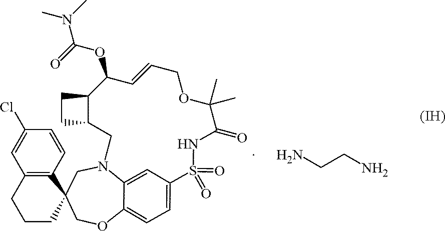
.

121. A crystalline form of the pharmaceutically acceptable salt of claim 120.

122. The crystalline form of claim 121, wherein said crystalline form is crystalline Form 1.

123. The crystalline form of claim 122, characterized by an X-ray powder diffraction pattern substantially as shown in FIG. 32.

124. The crystalline form of either claim 122 or claim 123, characterized by an X-ray powder diffraction pattern comprising peaks at 9.4, 10.6, 17.7, and 18.3 degrees ± 0.2 degrees 2-theta, on the 2-theta scale with lambda = 1.54 angstroms (Cu Kα).

125. The crystalline form of either claim 122 or claim 123, characterized by an X-ray powder diffraction pattern comprising peaks at 9.4, 10.6, 15.4, 17.7, and 18.3 degrees ± 0.2 degrees 2-theta, on the 2-theta scale with lambda = 1.54 angstroms (Cu Kα).

126. The crystalline form of either claim 122 or claim 123, characterized by an X-ray powder diffraction pattern comprising peaks at 9.4, 10.6, 15.4, 17.7, 18.3, 19.6, and 22.0 degrees ± 0.2 degree 2-theta, on the 2-theta scale with lambda = 1.54 angstroms (Cu Kα).

127. The crystalline form of either claim 122 or claim 123, characterized by an X-ray powder diffraction pattern comprising peaks at 9.4, 10.6, 15.4, 17.7, 18.3, 19.6, 22.0, 23.1, and 24.8 degrees ± 0.2 degree 2-theta, on the 2-theta scale with lambda = 1.54 angstroms (Cu Kα).

128. The crystalline form of either claim 122 or claim 123, characterized by an X-ray powder diffraction pattern comprising peaks at one or more of 9.4, 10.6, 15.4, 17.7, 18.3, 19.6, 22.0, 23.1, and 24.8 degrees ± 0.2 degrees 2-theta, on the 2-theta scale with lambda = 1.54 angstroms (Cu Kα).

129. The crystalline form of claim 121, wherein said crystalline form is crystalline Form 2.

130. The crystalline form of claim 129, characterized by an X-ray powder diffraction pattern substantially as shown in FIG. 32A.

131. The crystalline form of either claim 129 or claim 130, characterized by an X-ray powder diffraction pattern comprising peaks at 17.8 degrees ± 0.2 degrees 2-theta, on the 2-theta scale with lambda = 1.54 angstroms (Cu Kα).

132. The crystalline form of either claim 129 or claim 130, characterized by an X-ray powder diffraction pattern comprising peaks at 17.8, 21.8, 22.7, and 25.9 degrees ± 0.2 degrees 2-theta, on the 2-theta scale with lambda = 1.54 angstroms (Cu Kα).

133. The crystalline form of either claim 129 or claim 130, characterized by an X-ray powder diffraction pattern comprising peaks at 17.8, 21.8, 22.7, 25.9, and 29.5 degrees ± 0.2 degree 2-theta, on the 2-theta scale with lambda = 1.54 angstroms (Cu Kα).

134. The crystalline form of either claim 129 or claim 130, characterized by an X-ray powder diffraction pattern comprising peaks at 17.8, 21.8, 22.7, 25.9, 29.5, and 35.7 degrees ± 0.2 degree 2-theta, on the 2-theta scale with lambda = 1.54 angstroms (Cu Kα).

135. The crystalline form of either claim 129 or claim 130, characterized by an X-ray powder diffraction pattern comprising peaks at one or more of 17.8, 21.8, 22.7, 25.9, 29.5, and 35.7 degrees ± 0.2 degrees 2-theta, on the 2-theta scale with lambda = 1.54 angstroms (Cu Kα).

136. The pharmaceutically acceptable salt of claim 22, wherein the salt is the 4-((2-aminoethyl)amino)-4-methylpentan-2-one salt having Formula IK 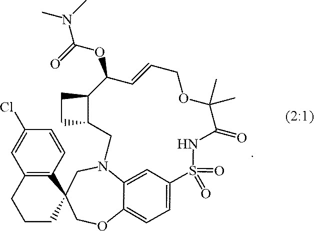 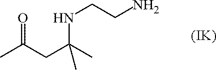
.

137. A crystalline form of the pharmaceutically acceptable salt of claim 136.

138. The crystalline form of claim 137, characterized by an X-ray powder diffraction pattern substantially as shown in FIG. 34.

139. The crystalline form of either claim 137 or claim 138, characterized by an X-ray powder diffraction pattern comprising peaks at 16.3, 17.2, and 18.0 degrees ± 0.2 degrees 2-theta, on the 2-theta scale with lambda = 1.54 angstroms (Cu Kα).

140. The crystalline form of either claim 137 or claim 138, characterized by an X-ray powder diffraction pattern comprising peaks at 7.3, 12.2, 12.8, 16.3, and 17.2 degrees ± 0.2 degrees 2-theta, on the 2-theta scale with lambda = 1.54 angstroms (Cu Kα).

141. The crystalline form of either claim 137 or claim 138, characterized by an X-ray powder diffraction pattern comprising peaks at 7.3, 12.2, 12.8, 16.3, 17.2, 18.0, 20.8, and 23.2 degrees ± 0.2 degree 2-theta, on the 2-theta scale with lambda = 1.54 angstroms (Cu Kα).

142. The crystalline form of either claim 137 or claim 138, characterized by an X-ray powder diffraction pattern comprising peaks at 7.3, 12.2, 12.8, 16.3, 17.2, 18.0, 20.8, 23.2, 24.3, and 26.6 degrees ± 0.2 degree 2-theta, on the 2-theta scale with lambda = 1.54 angstroms (Cu Kα).

143. The crystalline form of either claim 137 or claim 138, characterized by an X-ray powder diffraction pattern comprising peaks at one or more of 7.3, 12.2, 12.8, 16.3, 17.2, 18.0, 20.8, 23.2, 24.3, and 26.6 degrees ± 0.2 degrees 2-theta, on the 2-theta scale with lambda = 1.54 angstroms (Cu Kα).

144. The crystalline form of any one of claims 137 to 143, characterized by a differential scanning calorimetry (DSC) thermogram substantially as shown in FIG. 35 when heated at a rate of 10° C./min.

145. The crystalline form of any one of claims 137 to 144, characterized by a differential scanning calorimetry (DSC) thermogram comprising an endothermic peak at about 170° C. when heated at a rate of 10° C./min.

146. The crystalline form of any one of claims 137 to 145, characterized by a thermogravimetric analysis profile substantially as shown in FIG. 36 when heated at a rate of 20° C./min.

147. A pharmaceutical composition comprising a compound according to any one of claims 1 to 146 and a pharmaceutically acceptable excipient.

148. A method of inhibiting an MCL-1 enzyme comprising contacting the MCL-1 enzyme with an effective amount of a compound of any one of claims 1 to 146.

149. A method of treating a disease or disorder associated with aberrant MCL-1 activity in a subject comprising administering to the subject, a compound of any one of claims 1 to 146.

150. The method of claim 149, wherein the disease or disorder associated with aberrant MCL-1 activity is colon cancer, breast cancer, small-cell lung cancer, non-small-cell lung cancer, bladder cancer, ovarian cancer, prostate cancer, chronic lymphoid leukemia, lymphoma, myeloma, acute myeloid leukemia, or pancreatic cancer.
